(19) United States
(12) Patent Application Publication
(10) Pub. No.: US 2026/0176691 A1
(43) Pub. Date: Jun. 25, 2026

(54) METHOD AND KIT FOR MULTIPLEX QUANTITATIVE DETECTION OF MIRNAS FOR ALZHEIMER'S DISEASE

(71) Applicant: MIRACLE BIOTECHNOLOGY INC., Houston, TX (US)

(72) Inventors: CHANG SU, Clyde Hill, WA (US); YUAN CAO, Suzhou, Jiangsu (CN); DEMEI MA, Suzhou, Jiangsu (CN)

(21) Appl. No.: 18/836,925

(22) PCT Filed: May 26, 2023
(86) PCT No.: PCT/US2023/067522

§ 371 Date: Aug. 8, 2024

(30) Foreign Application Priority Data
May 6, 2023 (WO) PCT/CN2023/92571

(51) Int. Cl. C12Q 1/6883; C12Q 1/48; C12Q 1/6851; C12Q 1/686

(52) U.S. Cl. CPC C12Q 1/6883; C12Q 1/48; C12Q 1/6851; C12Q 1/686; C12Y 207/07; C12Q 2600/112; C12Q 2600/158; C12Q 2600/16; C12Q 2600/178

(57) ABSTRACT

A multiplex reverse transcription primer combination includes two or more stem-loop reverse transcription primers and demonstrates multi-specificity. The multiplex reverse transcription primer combination is capable of simultaneously quantifying two or more multiple target miRNAs including miR-16-5p, miR-34c-5p, miR-9-3p, and miR-9-5p.

### CROSS-REFERENCE TO RELATED PATENT APPLICATIONS

This application is a continuation in part application of PCT Patent Application PCT/CN2023/092571, filed on May 6, 2023, which is incorporated herein in its entirety by reference.

This application is related to co-pending PCT patent applications, entitled “Method for Screening Multiplex Reverse Transcription Primer Combinations for Quantifying Multiple miRNAs”, with Attorney Docket No. 1010469.100WO1, “Method and Kit for Multiplex Quantitative Detection of miRNAs for Lung Carcinoma”, with Attorney Docket No. 1010469.102WO1, “Multiplex-Reverse Transcription Primer Combinations for Multiplex Reverse Transcription—Quantitative PCR Reactions” with Attorney Docket No. 1010469.103WO1, and “Method for Clinical Detection of Multiple Target miRNAs”, with Attorney Docket No. 1010469.104WO1, which are filed on the same day that this application is filed, and with the same applicant as that of this application, any and all of which are incorporated respectively herein by reference in their entireties.

### STATEMENT REGARDING THE SEQUENCE LISTINGS

The Sequence Listings associated with this application are provided in xml format in lieu of a paper copy and are hereby incorporated by reference into the specification. The name of the xml file containing the Sequence Listings is 1010469_100WO1.xml. The xml file is about 225 KB, was created on Apr. 26, 2023.

### FIELD OF THE INVENTION

The present invention relates to a multiplex reverse transcription primer combination and its application in multiplex detection/quantification of multiple target miRNAs including miR-16-5p, miR-34c-5p, miR-9-3p, and miR-9-5p.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

Micro RNA or miRNA is a non-coding RNA widely existing in living organisms and generally has a length of 18-25 nucleotides. According to miRBase (ver. 22), human genome encodes approximately 2,600 mature miRNAs. Relevant studies have indicated that these miRNAs involve in numerous biological processes and their regulations, such as cell apoptosis, proliferation, organogenesis, development, tumorigenesis and hematopoiesis. Meanwhile, miRNA also regulates gene expressions in post-transcription stage, and thus plays an essential role for regulating multiple life courses such as, disease genesis and development, as well as cell senescence. Some studies have suggested that miRNA has the potential to be served as a tool for clinic diagonosis of diseases.

It wasn't until 1993 when Lee et al. cloned lin-4 using positional cloning from the nematode Caenorhabditis elegans, that the first miRNA was discovered. It was found that the lin-4 gene encodes for a miRNA of approximately 22 nucleotides in length. Later, in 2000, Reinhart et al. discovered let-7, which is the second gene known to regulate temporal development in the worm, and is a negative regulator that is also transcribed into a miRNA of 21 nucleotides. Since then, people have gradually realized that miRNAs are evolutionarily conserved important molecules that play a regulatory role in disease development and biological evolution.

Despite the potential for miRNAs to be used as a diagnostic tool for disease and their significant regulatory role, their accurate quantification is extremely difficult. Real-time fluorescent quantitative PCR (qPCR) detection is considered the gold standard for quantitative detection, but it was not applicable for miRNA quantification in the early days of research. This was due to the fact that miRNAs have very short sequences, low expression levels in cells, and highly homologous sequences. RNA blot hybridization and gene chips were the main methods used to study miRNA expressions in the early days, but these methods had limitations in terms of sample requirement, linear range, and sensitivity.

Until 2005, Chen et al. first implemented real-time fluorescent qPCR detection of a single miRNA using the stem-loop method (C. Chen, et al. (2005) Real-time quantification of microRNAs by stem-loop RT-PCR, Nucleic Acids Res., 33, e179). The detection process includes two main steps: reverse transcription and real-time PCR. In the first step, the stem-loop reverse transcription primer is mixed with the miRNA molecule, and a reverse transcription is performed using the MultiScribe™ reverse transcriptase. The resulting product is then quantitatively analyzed using conventional Taqman PCR. The stem-loop primer used has a 6-base complementary sequence to the 3′ end of the miRNA to initiate the reverse transcription reaction. In their experiment, it was observed that the stem-loop primer has better specificity and sensitivity compared to traditional linear primers. Nucleotide bases stacking can effectively improve thermal stability, and the spatial structure of the stem-loop sequence may facilitate its binding with double-stranded DNA molecules in the genome. They also suggested that the stem-loop reverse transcription primer has the potential to be used in multiple reverse transcription reactions and may have better efficiency and specificity.

To simultaneously detect/quantify multiple miRNAs in small samples, Tang et al. (2006) further improved the above method to achieve simultaneous detection of the expressions of 220 miRNAs in a single embryonic stem cell (Tang et al. (2006) MicroRNA expression profiling of single whole embryonic stem cells, Nucleic acids Res 24 e9). The method mainly involves reverse transcription of all miRNAs in a single embryonic stem cell under cyclic pulse temperature, followed by pre-PCR to increase the detection sensitivity of the resulting product, and then separate real-time fluorescence quantification of each miRNA expression. Lao et al. (2006) validated Tang's method and believed that as the number of detections increases, the mutual reaction between primers will also increase. The difference between the Ct values obtained for a certain miRNA under single and multiple detection conditions using the above method is observable. Therefore, the results obtained by this method still need to be verified under single detection conditions. Lao et al. also believed that this interaction between primers needs to be deeply analyzed and studied to be reduced, and this research requires testing a large number of primer combinations. Among the known 326 human miRNAs, this work is expensive and difficult for any laboratory. Currently, when studying the relationship between disease and the expression levels of multiple miRNAs, only Chen et al.'s single quantification detection method is used to fluorescence quantitatively detect miRNA expression levels separately.

As the number of detections increases, the mutual interaction between primers gradually increases, and its difficulty increases not only exponentially but also multiplicatively. Considering issues such as cross-reaction between probe primers, specificity between systems, sensitivity, and efficiency, it is almost impossible to achieve multiplex real-time fluorescence qPCR detection using a single principle. Current technology still cannot use the stem-loop reverse transcription primer method to achieve stable, standardized, and accurate multiple quantification detection of multiple miRNAs. These shortcomings also prevent it from entering the clinical field.

Therefore, there remains an imperative need for a multiplex detection method and multiplex stem-loop primer combinations which enable quantifying a combination of multiple target miRNAs simultaneously in a multiplex reverse transcription-fluorescent qPCR (RT-qPCR) process.

### SUMMARY OF THE INVENTION

In light of the foregoing, this invention discloses a multiplex stem-loop primer combination which enables quantifying a combination of multiple target miRNAs simultaneously in a multiplex RT-qPCR process.

In one aspect of the invention, a multiplex reverse transcription primer combination for simultaneously quantifying miR-16-5p, miR-34c-5p, miR-9-3p, and miR-9-5p, the multiplex reverse transcription primer combination comprising a first stem-loop reverse transcription primer comprising a nucleic acid sequence of SEQ ID No. 5; a second stem-loop reverse transcription primer comprising a nucleic acid sequence of SEQ ID No. 6; a third stem-loop reverse transcription primer comprising a nucleic acid sequence of SEQ ID No. 7; and a fourth stem-loop reverse transcription primer comprising a nucleic acid sequence of SEQ ID No. 8.

In another aspect of the invention, a multiplex reverse transcription primer combination for simultaneously quantifying a plurality of target miRNAs, the multiplex reverse transcription primer combination comprising a plurality of stem-loop reverse transcription primers; wherein each of the plurality of stem-loop reverse transcription primers has a stem-loop sequence forming a stem-loop structure and an anchor sequence complimentary to a unique 3′ sequence of one of the plurality of target miRNAs; wherein the lengths of the anchor sequencs of at least two of the stem-loop reverse transcription primers are different from each other.

In one embodiment, each of the plurality of stem-loop reverse transcription primers has a length of about 40-60 nt.

In one embodiment, the anchor sequence of each of the plurality of stem-loop reverse transcription primers has a length of about 3-12 nt.

In one embodiment, the plurality of stem-loop reverse transcription primers comprises a first stem-loop reverse transcription primer having a first stem-loop sequence and a first anchor sequence; and a second stem-loop reverse transcription primer having a second stem-loop sequence and a second anchor sequence, wherein each of the first and second stem-loop sequences is selected from the group consisting of SEQ ID Nos. 9-12; and the first and second stem-loop sequences are different from each other; and wherein the length of each of the first and second anchor sequences is selected from one of 4 nt, 6 nt, 8 nt, and 11 nt.

In one embodiment, the first anchor sequence of the first stem-loop reverse transcription primer is complimentary to a unique 3′ sequence of a first target miRNA of the plurality of target miRNAs and the second anchor sequence of the second stem-loop reverse transcription primer is complimentary to a unique 3′ sequence of a second target miRNA of the plurality of target miRNAs.

In one embodiment, the plurality of stem-loop reverse transcription primers further comprises a third stem-loop reverse transcription primer having a third stem-loop sequence and a third anchor sequence, wherein the third stem-loop sequence is selected from the group consisting of SEQ ID Nos. 9-12; and the third stem-loop sequence is different from the first and second stem-loop sequences; wherein the length of the third anchor sequence is selected from one of 4 nt, 6 nt, 8 nt, and 11 nt; and wherein the third anchor sequence is complimentary to a unique 3′ sequence of a third target miRNA of the plurality of target miRNAs.

In one embodiment, the plurality of stem-loop reverse transcription primers further comprises a fourth stem-loop reverse transcription primer having a fourth stem-loop sequence and a fourth anchor sequence, wherein the fourth stem-loop sequence is selected from the group consisting of SEQ ID Nos. 9-12; and the fourth stem-loop sequence is different from the first, second, and third stem-loop sequences; wherein the length of the fourth anchor sequence is selected from one of 4 nt, 6 nt, 8 nt, and 11 nt; and wherein the fourth anchor sequence is complimentary to a unique 3′ sequence of a fourth target miRNA of the plurality of target miRNAs.

In one embodiment, the multiplex reverse transcription primer combination has multi-specificity such that the first stem-loop reverse transcription primer effectively and only reverse transcribes the first target miRNA, the second stem-loop reverse transcription primer effectively and only reverse transcribes the second target miRNA, the third stem-loop reverse transcription primer effectively and only reverse transcribes the third target miRNA, and the fourth stem-loop reverse transcription primer effectively and only reverse transcribes the fourth target miRNA.

In one embodiment, each of the first, second, third and fourth target miRNAs is selected from the group consisting of miR-16-5p, miR-34c-5p, miR-9-3p, and miR-9-5p, wherein each of the first, second, third and fourth target miRNAs is different from the others.

In one embodiment, the first stem-loop reverse transcription primer comprises a nucleic acid sequence of SEQ ID No. 5, the second stem-loop reverse transcription primer comprises a nucleic acid sequence of SEQ ID No. 6, the third stem-loop reverse transcription primer comprises a nucleic acid sequence of SEQ ID No. 7, and the fourth stem-loop reverse transcription primer comprises a nucleic acid sequence of SEQ ID No. 8.

In another aspect of the invention, a kit for simultaneously quantifying expression level of a plurality of target miRNAs, the kit comprising a first stem-loop reverse transcription primer and a second stem-loop reverse transcription primer, wherein the first stem-loop reverse transcription primer has a first stem-loop sequence and a first anchor sequence, and the second stem-loop reverse transcription primer has a second stem-loop sequence and a second anchor sequence, wherein each of the first and second stem-loop sequences is selected from the group consisting of SEQ ID Nos. 9-12; and the first and second stem-loop sequences are different from each other, wherein the length of each of the first and second anchor sequences is selected from one of 4 nt, 6 nt, 8 nt, and 11 nt; first and second forward primers, wherein each of the first and second forward primers comprises a nucleic acid sequence selected from the group consisting of SEQ ID No. 17-20, and the first forward primer is different from the second forward primer; first and second reverse primers; wherein each of the first and second reverse primers comprises a nucleic acid sequence selected from the group consisting of SEQ ID No. 25-28, and the first reverse primer is different from the second reverse primer; and first and second probes, wherein the first probe comprises a first probe sequence, a first fluorescent reporter group, and a first quencher group; and the second probe comprises a second probe sequence, a second fluorescent reporter group, and a second quencher group, wherein each of the first and second probe sequences comprises a nucleic acid sequence selected from the group consisting of SEQ ID No. 21-24.

In one embodiment, each of the first and second fluorescent reporter groups is selected from the group consisting of VIC, CY5, ROX, and FAM; and the first fluorescent reporter group is different from the second fluorescent reporter group.

In one embodiment, the first anchor sequence is complimentary to a 3′ sequence of a first target miRNA of the plurality of target miRNAs, and the second anchor sequence is complimentary to a unique 3′ sequence of a second target miRNA of the plurality of target miRNAs.

In one embodiment, the kit further comprising a third stem-loop reverse transcription primer, wherein the third stem-loop reverse transcription primer has a third stem-loop sequence and a third anchor sequence, wherein the third stem-loop sequence is selected from the group consisting of SEQ ID Nos. 9-12; and the third stem-loop sequence is different from the first and second stem-loop sequences, and wherein the length of the third anchor sequence is selected from one of 4 nt, 6 nt, 8 nt, and 11 nt; a third forward primer, wherein the third forward primer comprises a nucleic acid sequence selected from the group consisting of SEQ ID Nos. 17-20; and the third forward primer is different from the first and second forward primers; a third reverse primer, wherein the third reverse primer comprises a nucleic acid sequence selected from the group consisting of SEQ ID Nos. 25-28; and the third reverse primer is different from the first and second reverse primers; and a third probe, wherein the third probe comprises a third probe sequence, a third fluorescent reporter group, and a third quencher group; wherein the third probe sequence comprises a nucleic acid sequence selected from the group consisting of SEQ ID Nos. 21-24.

In one embodiment, the third fluorescent reporter group is selected from the group consisting of VIC, CY5, ROX, and FAM; and the third fluorescent reporter group is different from the first and second fluorescent reporter groups.

In one embodiment, the kit further comprising a fourth stem-loop reverse transcription primer, wherein the fourth stem-loop reverse transcription primer has a fourth stem-loop sequence and a fourth anchor sequence, wherein the fourth stem-loop sequence is selected from the group consisting of SEQ ID Nos. 9-12; and the fourth stem-loop sequence is different from the first, second, and third stem-loop sequences, wherein the length of the fourth anchor sequence is selected from one of 4 nt, 6 nt, 8 nt, and 11 nt; a fourth forward primer, wherein the fourth forward primer comprises a nucleic acid sequence selected from the group consisting of SEQ ID Nos. 17-20; and the fourth forward primer is different from the first, second, and third forward primers; a fourth reverse primer, wherein the fourth reverse primer comprises a nucleic acid sequence selected from the group consisting of SEQ ID Nos. 25-28; and the fourth reverse primer is different from the first, second, and third reverse primers; and a fourth probe, wherein the fourth probe comprises a fourth probe sequence, a fourth fluorescent reporter group, and a fourth quencher group; wherein the fourth probe sequence comprises a nucleic acid sequence selected from the group consisting of SEQ ID Nos. 21-24.

In one embodiment, the fourth fluorescent reporter group is selected from the group consisting of VIC, CY5, ROX, and FAM; and the fourth fluorescent reporter group is different from the first, second, and third fluorescent reporter groups.

In another aspect of the invention, a kit for simultaneously quantifying expression level of a plurality of target miRNAs, the kit comprising a multiplex reverse transcription primer combination having a first stem-loop reverse transcription primer comprising a nucleic acid sequence of SEQ ID No. 5, a second stem-loop reverse transcription primer comprising a nucleic acid sequence of SEQ ID No. 6, a third stem-loop reverse transcription primer comprising a nucleic acid sequence of SEQ ID No. 7, and a fourth stem-loop reverse transcription primer comprising a nucleic acid sequence of SEQ ID No. 8; a forward primer combination having a first forward primer comprising a nucleic acid sequence of SEQ ID No. 17, a second forward primer comprising a nucleic acid sequence of SEQ ID No. 18, a third forward primer comprising a nucleic acid sequence of SEQ ID No: 19; and a fourth forward primer comprising a nucleic acid sequence of SEQ ID No. 20; a reverse primer combination having a first reverse primer comprising a nucleic acid sequence of SEQ ID No. 25, a second reverse primer comprising a nucleic acid sequence of SEQ ID No. 26, a third reverse primer comprising a nucleic acid sequence of SEQ ID No. 27; and a fourth reverse primer comprising a nucleic acid sequence of SEQ ID No. 28; and a probe combination having a first probe comprising a nucleic acid sequence of SEQ ID No. 21 and a first fluorescent reporter group of VIC, a second probe comprising a nucleic acid sequence of SEQ ID No. 22 and a second fluorescent reporter group of CY5, a third probe comprising a nucleic acid sequence of SEQ ID No. 23 and a third fluorescent reporter group of ROX, a fourth probe comprising a nucleic acid sequence of SEQ ID No. 24 and a fourth fluorescent reporter group of FAM.

In another aspect of the invention, a method of simultaneously quantifying miR-16-5p, miR-34c-5p, miR-9-3p, and miR-9-5p, the method comprising performing multiplex RT-qPCR using the multiplex reverse transcription primer combination disclsoured above.

In another aspect of the invention, a method for using quantification results of miR-16-5p, miR-34c-5p, miR-9-3p, and miR-9-5p for determining a medical condition of a living subject, the method comprising performing multiplex RT-qPCR using the kit of claim 19; obtaining quantification results of miR-16-5p, miR-34c-5p, miR-9-3p, and miR-9-5p from the multiplex RT-qPCR; and using the quantification results for determining a medical condition of the living subject, wherein the medical condition of the living subject is Alzheimer's Disease.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIGS. 1A-D show singleplex RT-qPCRs of individual target miRNA as templates. FIG. 1A shows the singleplex RT-qPCR of miR-16-5p/ddH₂O using its corresponding stem-loop reverse transcription primer with different anchor sequence lengths (6/8/11 nt). FIG. 1B shows the singleplex RT-qPCR of miR-34c-5p/ddH₂O using its corresponding stem-loop reverse transcription primer with different anchor sequence lengths (6/8/11 nt). FIG. 1C shows the singleplex RT-qPCR of miR-9-3p/ddH₂O using its corresponding stem-loop reverse transcription primer with different anchor sequence lengths (6/8/11 nt). FIG. 1D shows the singleplex RT-qPCR of miR-9-5p/ddH₂O using its corresponding stem-loop reverse transcription primer with different anchor sequence lengths (6/8/11 nt).

FIGS. 2A-F show results of validation on the specificity of the two duplex systems: miR-34c-5p and miR-9-3p; and miR-34c-5p and miR-16-5p. In particular, FIG. 2A shows amplification plots of the singleplex RT-qPCR using the selected miR-34c-5p's corresponding stem-loop reverse transcription primer for a mixed target miRNA template including miR-34c-5p and miR-9-3p. FIG. 2B shows amplification plots of the singleplex RT-qPCR using the selected miR-9-3p's corresponding stem-loop reverse transcription primer for a mixed target miRNA template including miR-34c-5p and miR-9-3p. FIG. 2C shows amplification plots of the singleplex RT-qPCR using the selected miR-34c-5p and miR-9-3p's corresponding stem-loop reverse transcription primers for ddH₂O. FIG. 2D shows amplification plots of the singleplex RT-qPCR using the selected miR-16-5p's corresponding stem-loop reverse transcription primer for a mixed target miRNA template including miR-34c-5p and miR-16-5p. FIG. 2E shows amplification plots of the singleplex RT-qPCR using the selected miR-34c-5p's corresponding stem-loop reverse transcription primer for a mixed target miRNA template including miR-34c-5p and miR-16-5p. FIG. 2F shows amplification plots of the singleplex RT-qPCR using the selected miR-34c-5p and miR-16-5p's corresponding stem-loop reverse transcription primers for ddH₂O.

FIGS. 3A-D show results of validation on the multi-specificity of two duplex systems: miR-34c-5p and miR-9-3p; and miR-34c-5p and miR-16-5p. In particular, FIG. 3A shows amplification plots of the duplex RT-qPCR using the selected miR-34c-5p's and miR-9-3p's corresponding stem-loop reverse transcription primers for a mixed target miRNA template including miR-34c-5p and miR-9-3p. FIG. 3B shows amplification plots of the duplex RT-qPCR using the selected miR-34c-5p's and miR-9-3p's corresponding stem-loop reverse transcription primers for ddH₂O. FIG. 3C shows amplification plots of the duplex RT-qPCR using the selected miR-34c-5p's and miR-16-5p's corresponding stem-loop reverse transcription primers for a mixed target miRNA template including miR-34c-5p and miR-16-5p. FIG. 3D shows amplification plots of the duplex RT-qPCR using the selected miR-34c-5p's and miR-16-5p's corresponding stem-loop reverse transcription primers for ddH₂O.

FIGS. 4A-D show results of sensitivity test of the two duplex systems: miR-34c-5p and miR-9-3p; and miR-34c-5p and miR-16-5p. In particular, FIG. 4A shows sensitivity test of miR-34c-5p. FIG. 4B shows sensitivity test of miR-9-3p. FIG. 4C shows sensitivity test of miR-34c-5p. FIG. 4D shows sensitivity test of miR-16-5p.

FIGS. 5A-F show a comparison between the singleplex quantification and duplex quantification of the two duplex systems: miR-34c-5p and miR-9-3p; and miR-34c-5p and miR-16-5p. In particular, FIG. 5A shows the singleplex quantification of miR-34c-5p. FIG. 5B shows the singleplex quantification of miR-9-3p. FIG. 5C shows the duplex quantification of miR-34c-5p and miR-9-3p. FIG. 5D shows the singleplex quantification of miR-34c-5p. FIG. 5E shows the singleplex quantification of miR-16c-5p. FIG. 5F shows the duplex quantification of miR-34c-5p and miR-16-5p.

FIGS. 6A-H show results of validation on the specificity of two triplex systems: miR-34c-5p, miR-9-3p, miR-16-5p; and miR-9-5p, miR-16-5p, miR-34c-5p. In particular, FIG. 6A shows amplification plots of the singleplex RT-qPCR using the selected miR-16-5p's corresponding stem-loop reverse transcription primer for a mixed target miRNA template including miR-34c-5p, miR-9-3p, miR-16-5p. FIG. 6B shows amplification plots of the singleplex RT-qPCR using the selected miR-9-3p's corresponding stem-loop reverse transcription primer for a mixed target miRNA template including miR-34c-5p, miR-9-3p, miR-16-5p. FIG. 6C shows amplification plots of the singleplex RT-qPCR using the selected miR-34c-5p's corresponding stem-loop reverse transcription primer for a mixed target miRNA template including miR-34c-5p, miR-9-3p, miR-16-5p. FIG. 6D shows amplification plots of the singleplex RT-qPCR using the selected miR-34c-5p's, miR-9-3p's and miR-16-5p's corresponding stem-loop reverse transcription primers for ddH₂O. FIG. 6E shows amplification plots of the singleplex RT-qPCR using the selected miR-9-5p's corresponding stem-loop reverse transcription primer for a mixed target miRNA template including miR-34c-5p, miR-9-5p, miR-16-5p. FIG. 6F shows amplification plots of the singleplex RT-qPCR using the selected miR-16-5p's corresponding stem-loop reverse transcription primer for a mixed target miRNA template including miR-34c-5p, miR-9-5p, miR-16-5p. FIG. 6G shows amplification plots of the singleplex RT-qPCR using the selected miR-34c-5p's corresponding stem-loop reverse transcription primer for a mixed target miRNA template including miR-34c-5p, miR-9-5p, miR-16-5p. FIG. 6H shows amplification plots of the singleplex RT-qPCR using the selected miR-34c-5p's, miR-9-5p's and miR-16-5p's corresponding stem-loop reverse transcription primers for ddH₂O.

FIGS. 7A-D show results of validation on the multi-specificity of the two triplex systems: miR-34c-5p, miR-16-5p and miR-9-3p; and miR-34c-5p, miR-16-5p and miR-9-5p. In particular, FIG. 7A shows amplification plots of the triplex RT-qPCR using the selected corresponding stem-loop reverse transcription primers of miR-34c-5p, miR-16-5p and miR-9-3p for a mixed target miRNA template including miR-34c-5p, miR-16-5p and miR-9-3p. FIG. 7B shows amplification plots of the triplex RT-qPCR using the selected corresponding stem-loop reverse transcription primers of miR-34c-5p, miR-16-5p and miR-9-3p for ddH₂O. FIG. 7C shows amplification plots of the triplex RT-qPCR using the selected corresponding stem-loop reverse transcription primers of miR-34c-5p, miR-16-5p and miR-9-5p for a mixed target miRNA template including miR-34c-5p, miR-16-5p and miR-9-5p. FIG. 7D shows amplification plots of the triplex RT-qPCR using the selected corresponding stem-loop reverse transcription primers of miR-34c-5p, miR-16-5p and miR-9-5p for ddH₂O.

FIGS. 8A-F show results of sensitivity test of the two triplex systems: miR-34c-5p, miR-16-5p and miR-9-3p; and miR-34c-5p, miR-16-5p and miR-9-5p. In particular, FIG. 8A shows sensitivity test of miR-9-3p in the triplex combination of miR-34c-5p, miR-16-5p and miR-9-3p. FIG. 8B shows sensitivity test of miR-16-5p in the triplex combination of miR-34c-5p, miR-16-5p and miR-9-3p. FIG. 8C shows sensitivity test of miR-34c-5p in the triplex combination of miR-34c-5p, miR-16-5p and miR-9-3p. FIG. 8D shows sensitivity test of miR-9-5p in the triplex combination of miR-34c-5p, miR-16-5p and miR-9-5p. FIG. 8E shows sensitivity test of miR-16-5p in the triplex combination of miR-34c-5p, miR-16-5p and miR-9-5p. FIG. 8F shows sensitivity test of miR-34c-5p in the triplex combination of miR-34c-5p, miR-16-5p and miR-9-5p.

FIGS. 9A-H show a comparison between the singleplex quantification and triplex quantification of the two triplex systems: miR-34c-5p, miR-16-5p and miR-9-3p; and miR-34c-5p, miR-16-5p and miR-9-5p. In particular, FIG. 9A shows the singleplex quantification of miR-34c-5p. FIG. 9B shows the singleplex quantification of miR-9-3p. FIG. 9C shows the singleplex quantification of miR-16-5p. FIG. 9D shows the triplex quantification of miR-34c-5p, miR-16-5p and miR-9-3p. FIG. 9E shows the singleplex quantification of miR-9-5p. FIG. 9F shows the singleplex quantification of miR-16-5p. FIG. 9G shows the singleplex quantification of miR-34c-5p. FIG. 9D shows the triplex quantification of miR-34c-5p, miR-16-5p and miR-9-5p.

FIGS. 10A-F show results of validation on the specificity of the quadruplex system: miR-9-3p, miR-9-5p, miR-16-5p, miR-34c-5p. In particular, FIG. 10A shows amplification plots of the singleplex RT-qPCR using the selected miR-9-3p's corresponding stem-loop reverse transcription primer. FIG. 10B shows amplification plots of the singleplex RT-qPCR using the selected miR-34c-5p's corresponding stem-loop reverse transcription primer. FIG. 10C shows amplification plots of the singleplex RT-qPCR using the selected miR-9-5p's corresponding stem-loop reverse transcription primer. FIG. 10D shows amplification plots of the singleplex RT-qPCR using the selected miR-16-5p's corresponding stem-loop reverse transcription primer.

FIG. 10E and FIG. 10F show amplification plots of the singleplex RT-qPCR using the selected miR-34c-5p's, miR-9-5p's, miR-9-3p's and miR-16-5p's corresponding stem-loop reverse transcription primers for ddH₂O.

FIGS. 11A-B show results of validation on the multi-specificity of the quadruplex system: miR-34c-5p, miR-16-5p, miR-9-5p, and miR-9-3p. In particular, FIG. 11A shows amplification plots of the quadruplex RT-qPCR using the selected corresponding stem-loop reverse transcription primers of miR-34c-5p, miR-16-5p, miR-9-5p, and miR-9-3p for a mixed target miRNA template including miR-34c-5p, miR-16-5p, mi-9-5p and miR-9-3p. FIG. 11B shows amplification plots of the quadruplex RT-qPCR using the selected corresponding stem-loop reverse transcription primers of miR-34c-5p, miR-16-5p, miR-9-3p, and miR-9-5p for ddH₂O.

FIGS. 12A-D show results of sensitivity test of the quadruplex system: miR-34c-5p, miR-16-5p, miR-9-5p and miR-9-3p. In particular, FIG. 12A shows sensitivity test of miR-9-5p in the quadruplex combination. FIG. 12B shows sensitivity test of miR-9-3p in the quadruplex combination. FIG. 12C shows sensitivity test of miR-16-5p in the quadruplex combination. FIG. 12D shows sensitivity test of miR-34c-5p in the quadruplex combination.

FIGS. 13A-F show a comparison between the singleplex quantification and quadruplex quantification of miR-34c-5p, miR-16-5p, miR-9-5p, and miR-9-3p. In particular, FIG. 13A shows the singleplex quantification of miR-34c-5p. FIG. 13B shows the singleplex quantification of miR-9-5p. FIG. 13C shows the singleplex quantification of miR-9-3p. FIG. 13D shows the singleplex quantification of miR-16-5p. FIG. 13E shows the quadruplex quantification of miR-34c-5p, miR-16-5p, miR-9-5p, and miR-9-3p. FIG. 13F shows a comparison between the singleplex quantification and quadruplex quantification of miR-34c-5p, miR-16-5p, miR-9-5p, and miR-9-3p.

FIGS. 14A-F show a stability test of the quadruplex quantification of miR-34c-5p, miR-16-5p, miR-9-5p, and miR-9-3p. In particular, FIG. 14A shows the singleplex quantification of miR-34c-5p. FIG. 14B shows the singleplex quantification of miR-9-5p. FIG. 14C shows the singleplex quantification of miR-9-3p. FIG. 14D shows the singleplex quantification of miR-16-5p. FIG. 14E shows the quadruplex quantification of miR-34c-5p, miR-16-5p, miR-9-5p, and miR-9-3p. FIG. 14F shows a comparison between the singleplex quantification and quadruplex quantification of miR-34c-5p, miR-16-5p, miR-9-5p, and miR-9-3p.

FIGS. 15A-D show cross-reactions within a quadruplex system comprising stem-loop reverse transcription primers each of which having a different anchor length. In particular, FIG. 15A shows amplification plots of the singleplex RT-qPCR using a miR-9-3p's stem-loop reverse transcription primer having an anchor sequence length of 8 nt. FIG. 15B shows amplification plots of the singleplex RT-qPCR using a miR-9-5p's stem-loop reverse transcription primer having an anchor sequence length of 6 nt. FIG. 15C shows amplification plots of the singleplex RT-qPCR using a miR-16-5p's stem-loop reverse transcription primer having an anchor sequence length of 11 nt. FIG. 15D shows amplification plots of the singleplex RT-qPCR using a miR-34c-5p's stem-loop reverse transcription primer having an anchor sequence length of 8 nt.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

As used in the disclosure, the term “target microRNA” or “target miRNA” refers to a microRNA sequence that is sought to be amplified and/or quantified. The target miRNA can be obtained from any source, and can comprise any number of different compositional components. The target microRNA can be a marker for a determining the condition of a subject. The condition can be a physiological or a mental condition. Further, it will be appreciated that “target miRNA” can refer to the target miRNA itself, as well as surrogates thereof, for example amplification products, and native sequences. The target miRNA of the present teachings can be derived from any of a number of sources, including without limitation, viruses, prokaryotes, eukaryotes, for example but not limited to plants, fungi, and animals. These sources may include, but are not limited to, whole blood, a tissue biopsy, lymph, bone marrow, amniotic fluid, hair, skin, semen, biowarfare agents, anal secretions, vaginal secretions, perspiration, saliva, buccal swabs, various environmental samples (for example, agricultural, water, and soil), research samples generally, purified samples generally, cultured cells, and lysed cells. It will be appreciated that target miRNA can be isolated from samples using any of a variety of procedures known in the art. In general, the target miRNA of the present teachings will be single stranded.

As used in the disclosure, the term “reverse transcription reaction” refers to an elongation reaction in which the 3′ target-specific portion of a stem-loop primer is extended to form an extension reaction product comprising a strand complementary to the target miRNA. In some embodiments, the target miRNA is a miRNA molecule and the extension reaction is a reverse transcription reaction comprising a reverse transcriptase, where the 3′ end of a stem-loop primer is extended. In some embodiments, the extension reaction is a reverse transcription reaction comprising a polymerase derived from a Eubacteria. In some embodiments, the extension reaction can comprise rTth polymerase, for example as commercially available from Applied Biosystems catalog number N808-0192, and N808-0098. In some embodiments, the target miRNA is a miRNA or other RNA molecule, and the use of polymerases that also comprise reverse transcription properties can allow for a first reverse transcription reaction followed thereafter by an amplification reaction such as a multi-plexed PCR-based pre-amplification in the same reaction vessel, thereby allowing for the consolidation of two reactions in single reaction vessel. In some embodiments, the target miRNA is a DNA molecule and the extension reaction comprises a polymerase and results in the synthesis of a complementary strand of DNA. The term reverse transcription also includes also includes the synthesis of a DNA complement of a template DNA molecule. Similarly, a reverse transcription product can be a DNA molecule synthesized in a reverse transcription reaction, which is thus complementary to the template.

As used in the disclosure, the term “reverse transcription reaction” refers to an elongation reaction in which the 3′ target-specific portion of a stem-loop primer is extended to form an extension reaction product comprising a strand complementary to the target miRNA. In some embodiments, the extension reaction is a reverse transcription reaction comprising a reverse transcriptase, where the 3′ end of a stem-loop primer is extended. In some embodiments, the extension reaction is a reverse transcription reaction comprising a polymerase derived from a Eubacteria. In some embodiments, the extension reaction can comprise rTth polymerase. In some embodiments, the use of polymerases that also comprise reverse transcription properties can allow for a first reverse transcription reaction followed thereafter by an amplification reaction such as a multiplex fluorescent quantitative PCR in the same reaction vessel, thereby allowing for the consolidation of two reactions in single reaction vessel. A reverse transcription product can be a DNA molecule synthesized in a reverse transcription reaction, which is thus complementary to the target miRNA template.

As used in the disclosure, the term “hybridization” refers to the complementary base-pairing interaction of one nucleic acid with another nucleic acid that results in the formation of a duplex, triplex, or other higher-ordered structure, and is used herein interchangeably with “annealing.” Typically, the primary interaction is base specific, e.g., A/T and G/C, by Watson/Crick and Hoogsteen-type hydrogen bonding. Base-stacking and hydrophobic interactions can also contribute to duplex stability. Conditions for hybridizing primers to complementary and substantially complementary target sequences are well known, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the polynucleotides and the complementary, the pH, the temperature, the presence of mono- and divalent cations, the proportion of G and C nucleotides in the hybridizing region, the viscosity of the medium, and the presence of denaturants. Such variables influence the time required for hybridization. Thus, the preferred annealing conditions will depend upon the particular application. Such conditions, however, can be routinely determined by the person of ordinary skill in the art without undue experimentation. It will be appreciated that complementarity need not be perfect; there can be a small number of base pair mismatches that will minimally interfere with hybridization between the target sequence and the single stranded nucleic acids of the present teachings. However, if the number of base pair mismatches is so great that no hybridization can occur under minimally stringent conditions then the sequence is generally not a complementary target sequence. Thus, complementarity herein is meant that primers are sufficiently complementary to the target sequence to hybridize under the selected reaction conditions to achieve the ends of the present teachings.

As used in the disclosure, the term “amplifying” refers to any means by which at least a part of a target miRNA or its surrogate is reproduced, typically in a template-dependent manner, including without limitation, a broad range of techniques for amplifying nucleic acid sequences, either linearly or exponentially. In some embodiments, amplification can be achieved in a self-contained integrated approach comprising sample preparation and detection, as described for example in U.S. Pat. Nos. 6,153,425 and 6,649,378. Amplifying nucleic acids can employ reversibly modified enzymes, for example but not limited to those described in U.S. Pat. No. 5,773,258. The present teachings also contemplate various uracil-based decontamination strategies, wherein for example uracil can be incorporated into an amplification reaction, and subsequent carry-over products removed with various glycosylase treatments (see for example U.S. Pat. No. 5,536,649. Those in the art will understand that any protein with the desired enzymatic activity can be used in the disclosed methods and kits.

As used in the disclosure, the term “fluorescent quantitative PCR”, “quantitative PCR”, or “qPCR” refers to a PCR reaction performed in such a way and under such controlled conditions that the results of the assay are quantitative, that is, the assay is capable of quantifying the amount or concentration of a nucleic acid ligand present in the test sample. qPCR is a technique based on the polymerase chain reaction, and is used to amplify and simultaneously quantify a targeted nucleic acid molecule. qPCR allows for both detection and quantification (as absolute number of copies or relative amount when normalized to DNA input or additional normalizing genes) of a specific sequence in a DNA sample. In some embodiments, the DNA sample is a products containing cDNA produced from reverse transcription of a RNA sample, e.g. miRNA. The procedure follows the general principle of PCR, with the additional feature that the amplified DNA is quantified as it accumulates in the reaction in real time after each amplification cycle. qPCR is described, for example, in Kurnit et al. (U.S. Pat. No. 6,033,854), Wang et al. (U.S. Pat. Nos. 5,567,583 and 5,348,853), Ma et al. (The Journal of American Science, 2 (3), (2006)), Heid et al. (Genome Research 986-994, (1996)), Sambrook and Russell (Quantitative PCR, Cold Spring Harbor Protocols, (2006)), and Higuchi (U.S. Pat. Nos. 6,171,785 and 5,994,056). The contents of these are incorporated by reference herein in their entirety.

As used in the disclosure, the term “detection” refers to any of a variety of ways of determining the presence and/or quantity and/or identity of a target miRNA. In some embodiments employing a donor moiety and signal moiety, one may use certain energy-transfer fluorescent dyes. Certain nonlimiting exemplary pairs of donors (donor moieties) and acceptors (signal moieties) are illustrated, e.g., in U.S. Pat. Nos. 5,863,727; 5,800,996; and 5,945,526. Use of some combinations of a donor and an acceptor have been called FRET (Fluorescent Resonance Energy Transfer). In some embodiments, fluorophores that can be used as signaling probes include, but are not limited to, rhodamine, cyanine 3 (Cy 3), cyanine 5 (Cy 5), fluorescein, Vic™, LiZ™, Tamra™, 5-Fam™, 6-Fam™, and Texas Red (Molecular Probes). (Vic™, Liz™, Tamra™, 5-Fam™, and 6-Fam™. In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator. According to some embodiments, one can employ an internal standard to quantify the amplification product indicated by the fluorescent signal. See, e.g., U.S. Pat. No. 5,736,333. Devices have been developed that can perform a thermal cycling reaction with compositions containing a fluorescent indicator, emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; and 6,174,670. In some embodiments, combined thermal cycling and fluorescence detecting devices can be used for precise quantification of target nucleic acid sequences in samples. In some embodiments, fluorescent signals can be detected and displayed during and/or after one or more thermal cycles, thus permitting monitoring of amplification products as the reactions occur in “real time.” In some embodiments, one can use the amount of amplification product and number of amplification cycles to calculate how much of the target nucleic acid sequence was in the sample prior to amplification. In some embodiments, one could simply monitor the amount of amplification product after a predetermined number of cycles sufficient to indicate the presence of the target nucleic acid sequence in the sample. One skilled in the art can easily determine, for any given sample type, primer sequence, and reaction condition, how many cycles are sufficient to determine the presence of a given target miRNA. As used herein, determining the presence of a target can comprise identifying it, as well as optionally quantifying it. In some embodiments, the amplification products can be scored as positive or negative as soon as a given number of cycles is complete. In some embodiments, the results may be transmitted electronically directly to a database and tabulated. Thus, in some embodiments, large numbers of samples can be processed and analyzed with less time and labor when such an instrument is used. In some embodiments, different detector probes may distinguish between different target miRNAs. A non-limiting example of such a probe is a 5′-nuclease fluorescent probe, such as a TaqMan® probe molecule or MGB probe, wherein a fluorescent molecule is attached to a fluorescence-quenching molecule through an oligonucleotide link element. In some embodiments, the oligonucleotide link element of the 5′-nuclease fluorescent probe binds to a specific sequence of an identifying portion or its complement. In some embodiments, different 5′-nuclease fluorescent probes, each fluorescing at different wavelengths, can distinguish between different amplification products within the same amplification reaction. For example, in some embodiments, one could use two different 5′-nuclease fluorescent probes that fluoresce at two different wavelengths (WLA and WLB) and that are specific to two different regions of two different extension reaction products (A and B, respectively). Amplification product A is formed if target miRNA A is in the sample, and amplification product B is formed if target polynucleotide B is in the sample. After amplification, one can determine which specific target nucleic acid sequences are present in the sample based on the wavelength of signal detected and their intensity. Thus, if an appropriate detectable signal value of only wavelength WLA is detected, one would know that the sample includes target miRNA A, but not target miRNA B. If an appropriate detectable signal value of both wavelengths WLA and WLB are detected, one would know that the sample includes both target miRNA A and target miRNA B.

As used in the disclosure, the term “detector probe” or “probe” refers to a molecule used in an amplification reaction, typically for quantitative or real-time PCR analysis, as well as end-point analysis. Such probes can be used to monitor the amplification of products of reverse transcription of the target micro RNAs. In some embodiments, probes present in an amplification reaction are suitable for monitoring the amount of amplicon(s) produced as a function of time. Such detector probes include, but are not limited to, the 5′-exonuclease assay (TaqMan® probes described herein (see also U.S. Pat. No. 5,538,848) various stem-loop molecular beacons (see e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, e.g., Kubista et al., 2001, SPIE 4264:53-58), non-FRET probes (see, e.g., U.S. Pat. No. 6,150,097), Sunrise®/Amplifluor® probes (U.S. Pat. No. 6,548,250), stem-loop and duplex Scorpion™ probes (Solinas et al., 2001, Nucleic Acids Research 29: E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Probes can also comprise quenchers, including without limitation black hole quenchers (Biosearch), Iowa Black (IDT), QSY quencher (Molecular Probes), and Dabsyl and Dabcel sulfonate/carboxylate Quenchers (Epoch). Probes can also comprise two probes, wherein for example a fluor is on one probe, and a quencher is on the other probe, wherein hybridization of the two probes together on a target quenches the signal, or wherein hybridization on the target alters the signal signature via a change in fluorescence. Probes can also comprise sulfonate derivatives of fluorescenin dyes with S03 instead of the carboxylate group, phosphoramidite forms of fluorescein, phosphoramidite forms of CY 5 (commercially available for example from Amersham). In some embodiments, intercalating labels are used such as ethidium bromide, SYBR® Green I (Molecular Probes), and PicoGreene® (Molecular Probes), thereby allowing visualization in real-time, or end point, of an amplification product in the absence of a probe. In some embodiments, real-time visualization can comprise both an intercalating probe and a sequence-based detector probe can be employed. In some embodiments, the detector probe is at least partially quenched when not hybridized to a complementary sequence in the amplification reaction, and is at least partially unquenched when hybridized to a complementary sequence in the amplification reaction. In some embodiments, probes can further comprise various modifications such as a minor groove binder (see for example U.S. Pat. No. 6,486,308) to further provide desirable thermodynamic characteristics. In some embodiments, detector probes can correspond to the zip-code introduced by the stem-loop reverse transcription primer.

As used in the disclosure, the term “stem-loop primer” or “stem-loop reverse transcription primer” refers to a molecule comprising an anchor sequence on its 3′end and a stem-loop structure on its 5′ end. The stem-loop structure comprises a stem portion and a loop portion. The term “anchor sequence” refers to the single stranded portion of a stem-loop primer that is complementary to a target miRNA. The anchor sequence is located downstream from the stem-loop structure of the stem-loop primer. Generally, the anchor sequence is between 3 and 12 nucleotides long. The term “stem” refers to the double stranded region of the stem-loop structure of the stem-loop primer that is between the anchor sequence and the loop, and is discussed more fully below. The term “loop” refers to a region of the stem-loop sequence that is located between the two complementary strands of the stem. Typically, the loop comprises single stranded nucleotides, though other moieties including modified RNA, Carbon spacers such as C18, and/or PEG (polyethylene glycol) are also possible. Generally, the loop is between 4 and 30 nucleotides long. In some embodiments, the loop is between 14 and 18 nucleotides long. In some embodiments, the loop is 16 nucleotides long. Those in the art will appreciate that loops shorter that 4 nucleotides and longer than 20 nucleotides can be identified in the course of routine methodology and without undue experimentation, and that such shorter and longer loops are contemplated by the present teachings.

As used in the disclosure, the term “Tm-enhancing tail” refers to a small number of nucleobases, typically between 3 and 10, that are included at the 5′ end of the forward primer used in the qPCR reaction. The Tm-enhancing tail is not complementary to the reverse transcription product. In some embodiments, the Tm-enhancing tail is 4 bases. In some embodiments, the Tm-enhancing tail is 5 bases. In some embodiments, the Tm-enhancing tail is 6 bases. In some embodiments, the Tm-enhancing tail is 7 bases. Generally, longer Tm enhancing tails are possible, but will come at the cost of increased expense in oligonucleotide manufacturing, will further add to reaction complexity, and may raise the Tm to undesirable levels.

As used in the disclosure, “multiplex” refers to a reaction in which multiple targets DNA/RNA and/or targets in or from multiple samples are transcribed, amplified or quantified in the same reaction. In some embodiments of any of the aspects, a multiplex reverse transcription reaction can comprise reverse transcription of 1 to 100 target nucleotide sequences. As a non-limiting example, a multiplex reverse transcription reaction can comprise reverse transcription of about 1 sample, about 2 samples, about 3 samples, about 4 samples, about 5 samples, about 6 samples, about 7 samples, about 8 samples, about 9 samples, about 10 samples, about 20 samples, about 30 samples, about 40 samples, about 50 samples, about 60 samples, about 70 samples, about 80 samples, about 90 samples, about 100 samples. “singleplex” refers to a reaction in which only one target DNA/RNA is transcribed, amplified or quantified in the same reaction.

As used in the disclosure, a “fragment” or “portion” of a nucleotide sequence refers to a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of and/or consisting of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 990/identical) to the reference nucleic acid or nucleotide sequence. Such a nucleic acid fragment or portion according to the invention may be, where appropriate, included in a larger polynucleotide of which it is a constituent. Thus, hybridizing to (or hybridizes to, and other grammatical variations thereof), for example, at least a portion of a target miRNA or cDNA, refers to hybridization to a nucleotide sequence that is identical or substantially identical to a length of contiguous nucleotides of the target miRNA or cDNA.

As used in the disclosure, a “heterologous” or a “recombinant” nucleotide sequence is a nucleotide sequence not naturally associated with a host cell into which it is introduced, including non-naturally occurring multiple copies of a naturally occurring nucleotide sequence. Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to said nucleotide sequence of the invention.

As used in the disclosure, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used in the disclosure, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90°%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 5 residues to about 150 residues in length. Thus, in some embodiments of the invention, the substantial identity exists over a region of the sequences that is at least about 3 to about 15 (e.g., 3, 4, 5, 6, 7, 8, 9, 10, 11, 12, 13, 14, 15 residues in length and the like or any value or any range therein), at least about 2 to about 30, at least about 5 to about 30, at least about 10 to about 30, at least about 16 to about 30, at least about 18 to at least about 25, at least about 18, at least about 22, at least about 25, at least about 30, at least about 40, at least about 50, about 60, about 70, about 80, about 90, about 100, about 110, about 120, about 130, about 140, about 150, or more residues in length, and any range therein. In representative embodiments, the sequences can be substantially identical over at least about 15 nucleotides. In some particular embodiments, the sequences are substantially identical over at least about 150 residues. In some embodiments, sequences of the invention can be about 70°% to about 100% identical over at least about 15 nucleotides to about 25 nucleotides. In some embodiments, sequences of the invention can be about 75% to about 100% identical over at least about 15 nucleotides to about 25 nucleotides. In further embodiments, sequences of the invention can be about 80% to about 100% identical over at least about 15 nucleotides to about 25 nucleotides. In further embodiments, sequences of the invention can be about 80% to about 100% identical over at least about 7 nucleotides to about 25 nucleotides. In some embodiments, sequences of the invention can be about 70% identical over at least about 15 nucleotides. In other embodiments, the sequences can be about 85% identical over about 22 nucleotides. In still other embodiments, the sequences can be 100% homologous over about 15 nucleotides. In a further embodiment, the sequences are substantially identical over the entire length of the coding regions. Furthermore, in representative embodiments, substantially identical nucleotide or protein sequences perform substantially the same function, e.g. reverse transcription.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues: always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Two nucleotide sequences can also be considered to be substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tm) for the specific sequence at a defined ionic strength and pH.

The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see. Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The multiplex real-time fluorescence qPCR (or, qPCR) detection/quantification described in the present invention refers to a process, in which, in one PCR tube, two or more qPCR systems are configured to perform qPCR reactions simultaneously, and thus double or multiple real-time fluorescence quantifications are conducted together.

The present invention provides a multiplex quantification method for miR-16-5p, miR-34c-5p, miR-9-3p, and miR-9-5p. The method includes two steps. The first step involves reverse transcription of each target miRNA in the sample using stem-loop reverse transcription primers. The stem-loop sequences in each reverse transcription primer are different. Then, in the second step, multiplex qPCR detection/quantification is performed on all reverse transcription products together. In the reverse transcription of the first step, miR-16-5p is reverse transcribed using the first stem-loop reverse transcription primer, which has 8 anchor bases. miR-34c-5p is reverse transcribed using the second stem-loop reverse transcription primer, which has 6 anchor bases. miR-9-3p is reverse transcribed using the third stem-loop reverse transcription primer, which has 6 anchor bases. miR-9-5p is reverse transcribed using the fourth stem-loop reverse transcription primer, which has 11 anchor bases.

The present invention also provides a multiple quantification/detection kit for miR-16-5p, miR-34c-5p, miR-9-3p, and miR-9-5p, including reverse transcription reagents. The reverse transcription reagents include two or more of the first, second, third, and fourth stem-loop reverse transcription primers, among which the first stem-loop reverse transcription primer is used for reverse transcription of miR-16-5p and contains 8 anchor bases. The second stem-loop reverse transcription primer is used for reverse transcription of miR-34c-5p and contains 6 anchor bases. The third stem-loop reverse transcription primer is used for reverse transcription of miR-9-3p and contains 6 anchor bases. The fourth stem-loop reverse transcription primer is used for reverse transcription of miR-9-5p and contains 11 anchor bases.

The length of the anchor sequence in the stem-loop reverse transcription primer is believed to be related to the efficiency of reverse transcription and the specificity of the stem-loop reverse transcription primer itself. Surprisingly, it has been found in the present invention that when quantifying multiple miRNAs, a combination of stem-loop reverse transcription primers, in which each of the stem-loop reverse transcription primers has a specific anchor sequence length demonstrates outstanding multi-specificity. That is, each of the stem-loop reverse transcription primers effectively and only reverse transcribing the target miRNA to which its anchor sequence is complimentary.

As a result, in one PCR tube, the multiplex RT can be performed on the sample containing the multiple target miRNAs with a combination of the stem-loop reverse transcription primers, and the multiplex qPCR can then be performed in the same PCR tube. The quantification results obtained by conducting the multiplex RT-qPCR in the same tube are substantially same to those of singleplex RT-singleplex qPCR detection in terms of amplification curves and Ct values. Therefore, the present invention can be used in clinical diagnosis of diseases.

### Example

The Target miRNAs

In this embodiment, target miRNAs, which relate to Alzheimer's disease, are quantified using a selected multiplex stem-loop reverse transcription primer combination in a multiplex RT-qPCR process. In one embodiment, the target miRNAs includes miR-16-5p, miR-34c-5p, miR-9-3p, and miR-9-5p. The nucleotide sequences of all four miRNAs are reported in the microRNA database www.mirbase.org. Specifically, the sequence of miR-16-5p is shown as SEQ ID No. 1, the sequence of miR-34c-5p is shown as SEQ ID No. 2, the sequence of miR-9-3p is shown as SEQ ID No. 3, and the sequence of miR-9-5p is shown as SEQ ID No. 4.

### RNA Templates and Mixed RNA Templates

Each single target miRNA template includes single synthesis RNA templates of each of miR-16-5p, miR-34c-5p, miR-9-3p, and miR-9-5p, respectively. The mixed target miRNAs templates include a mixture of two or more single target miRNA templates at a ratio desired.

### Principle for Designing Stem-Loop Reverse Transcription Primers

The design principles for the stem-loop reverse transcription primers are described as follows. From its 5′ to 3′ end, a stem-loop reverse transcription primer sequentially comprises a 5′ end, a stem-loop sequence, an anchor base sequence, and a 3′ end. The number of nucleotide bases in each of the stem-loop reverse transcription primers used is generally between 40 and 65, with a double-stranded region formed in the stem-loop sequence by a pair of fragments complimentary to each other. This double stranded region is the “stem”, and typically has 11-14 paired nucleotide bases. The unpaired region, located between the pair of complimentary fragments, protrudes to form a “loop,” which is typically a sequence of 16-36 nucleotide bases. In one embodiment, the stem-loop reverse transcription primers with different stem-loop sequences are designed, and their stem-loop sequences are as follows in Table 1.

TABLE 1
Stem-loop sequences for stem-loop reverse transcription primers

| Name | Sequence |
|---|---|
| Stem-loop structure 1 | SEQ ID No. 9 |
| Stem-loop structure 2 | SEQ ID No. 10 |
| Stem-loop structure 3 | SEQ ID No. 11 |
| Stem-loop structure 4 | SEQ ID No. 12 |
| Stem-loop structure 5 | SEQ ID No. 37 |

Oligo Analyzer (idtdna.com) is used to analyze and compare the differences and interference between the stem-loop sequences of the designed stem-loop reverse transcription primers. In this embodiment, a stem-loop reverse transcription primer containing the stem-loop structure 1 (SEQ ID No. 9) is selected for the quantification/detection of miR-16-5p using the multiplex RT-qPCR; a stem-loop reverse transcription primer containing the stem-loop structure 2 (SEQ ID No. 10) is selected for the quantification/detection of miR-34c-5p using the multiplex RT-qPCR; a stem-loop reverse transcription primer containing the stem-loop structure 3 (SEQ ID No. 11) is selected for the quantification/detection of miR-9-3p using the multiplex RT-qPCR; a stem-loop reverse transcription primer containing the stem-loop structure 4 (SEQ ID No. 12) is selected for the quantification/detection of miR-9-5p using the multiplex RT-qPCR. Table 2 reflects the stem-loop sequence selected for each stem-loop reverse transcription primer.

TABLE 2
Stem-loop sequence selected for each stem-
loop reverse transcription primer

| Name | Sequence | Remarks |
|---|---|---|
| Stem | SEQ ID | Located on the stem-loop reverse |
| loop 1 | No. 9 | transcription primer for miR-16-5p |
| Stem | SEQ ID | Located on the stem-loop reverse |
| loop 2 | No. 10 | transcription primer for miR-34c-5p |
| Stem | SEQ ID | Located on the stem-loop reverse |
| loop 3 | No. 11 | transcription primer for miR-9-3p |
| Stem | SEQ ID | Located on the stem-loop reverse |
| loop 4 | No. 12 | transcription primer for miR-9-5p |

The anchor sequence of the stem-loop reverse transcription primer locates downstream of the stem-loop sequence. The anchor sequence is complimentary to a 3′ sequence of the target miRNA of which the stem-loop reverse transcription primer is designed for reverse transcription. The anchor sequence typically has a length between 3-12 nt. In one embodiment, the anchor sequence has a length between 4-11 nt. In one embodiment, the anchor sequence has a length between 4-8 nt. In one embodiment, the anchor sequence has a length between 6-11 nt. In one embodiment, the anchor sequence has a length between 6-8 nt.

### Principles for Designing Forward Primers

The 5′ end to 3′ end of the forward primer is: 5′-Tm-enhancing tail-specific complimentary sequence-3′, where the Tm-enhancing tail comprises randomly arranged A, T, C, or G to increase the Tm value of the forward primer. The GC content of the forward primer is generally between 50% to 60%, and the 3′ end of primer should avoid having three or more consecutive G or C bases. The length of the Tm-enhancing tail of the forward primer should be selected to ensure that the difference between the Tm value of the forward primer and the reverse primer should be within 1° C., and both of the Tm values of the forward and reverse primer should be around 55-60° C. The designed forward primers are shown in Table 3.

TABLE 3
Sequences of forward primers for each target miRNA in qPCR

| Name | Sequence |
|---|---|
| miR-16-5p-F | SEQ ID No. 17 |
| miR-34c-5p-F | SEQ ID No. 18 |
| miR-9-3p-F | SEQ ID No. 19 |
| miR-9-5p-F | SEQ ID No. 20 |

The forward primers are named in a format of: target miRNA-F. For example, if a forward primer is named as miR-16-5p-F, the forward primer is used for the qPCR amplification of miR-16-5p.

### Principles for Designing Reverse Primers

The length of the reverse primer is generally about 15-25 nt, with a Tm value of 55-60° C. and a GC content of 50%-60%. The 3′ end of the primer should avoid having three or more consecutive G or C bases, and the Tm value of the forward and reverse primers should be within 1° C. of each other. To increase amplification efficiency, the reverse primer should be designed as close as possible to the loop of the stem-loop structure. The sequences of the designed reverse primers are shown in Table 4.

TABLE 4
Sequences of reverse primers for each target miRNA in qPCR

| Name | Sequence |
|---|---|
| miR-16-5p-R | SEQ ID No. 25 |
| miR-34c-5p-R | SEQ ID No. 26 |
| miR-9-3p-R | SEQ ID No. 27 |
| miR-9-5p-R | SEQ ID No. 28 |

The reverse primers are named in a format of: target miRNA-R. For example, if a reverse primer is named as miR-16-5p-R, the reverse primer is used for qPCR amplification of miR-16-5p.

### Principles for Designing Probes

In one embodiment, Taqman® probes are used as the probes in the qPCR reaction. The length of each probe generally ranges from 25 nt to 32 nt, and the Tm value is between 65-75° C., which is 5-10° C. higher than that of the forward/reverse primers. The GC content is 30-80%. The first base at the 5′ end of each of the probes cannot be G, and should be as close as possible to the 3′ end of the target miRNA sequence. The 5′ end of the probe is labeled with a fluorescent reporter group, and the 3′ end is labeled with a quencher group. The fluorescent reporter group may be selected from fluorescent reporter groups such as FAM, VIC, CY5 or ROX. The designed probes are shown in Table 5.

TABLE 5
Sequences of probes in qPCR

| | | Fluorescent | Quencher |
|---|---|---|---|
| | | reporter group | group |
| Name | Sequence | on the 5′ | on the 3′ |
| miR-16-5p-VIC | SEQ ID No. 21 | VIC | BHQ1 |
| miR-34c-5p-CY5 | SEQ ID No. 22 | CY5 | BHQ3 |
| miR-9-3p-ROC | SEQ ID No. 23 | ROC | BHQ2 |
| miR-9-5p-FAM | SEQ ID No. 24 | FAM | BHQ1 |

The probes are named in a format of: target miRNA-fluorescent reporter group. For example, if a probe is named as miR-16-5p-VIC, the probe is used for detection of miR-16-5p in the qPCR, and the VIC is used as the fluorescent reporter group on the 5′ end of the probe.

In one embodiment, in the above-mentioned multiplex qPCR, the design of each forward primer and each probe should satisfy the following principles: The 3′ end of each forward primer sequence has a Tm-enhancing sequence with a length of less than 8 nt. The 5′ end of each forward primer sequence has a sequence that is identical to a DNA equivalent (replacing any U of the target miRNA with a T) of a 5′ end sequence of the target miRNA and has a length between 9-13 nt. The 5′ end of each probe sequence has a sequence that is identical to a DNA equivalent of a 3′ end sequence of the target miRNA and has a length between 9-12 nt. The 3′ end of each probe sequence has a sequence that is identical to a 5′ end sequence of the stem-loop sequence and has a length greater than 6 nt.

The above-mentioned forward/reverse primers and probes are synthesized by Hippobio Ltd., Huzhou, Zhejiang, China.

Extraction of miRNA Samples from Biological Samples

In one embodiment, the peripheral blood is used as a biological sample for miRNA extraction. It should be noted that other biological samples, e.g. cerebrospinal fluid, epithelial cells, or bones maybe used as the biological samples for the miRNAs' extraction.

Blood serum processing: With the informed consent of all subjects, 5 mL of peripheral blood was collected from each participant using a coagulation-promoting separation gel vacuum blood collection tube. After standing for 30 minutes, the sample was centrifuged at room temperature at 3000×g for 10 minutes, and the upper layer serum was collected and stored at −80° C. for later use.

Extraction of serum miRNA: Qiagen serum/plasma miRNA extraction kit was used with the column-based nucleic acid extraction method. 200 μL of serum was used according to the instructions of the kit, followed by the addition of 20 μL of ddH₂O for dissolving. The extracted miRNA was stored at −80° C. for later use.

### Reverse Transcription System and Process

1 μL of mixed target miRNA template or water was mixed with 2 μL of 10×RT MIX, 2 μL of HiScript II Enzyme Mix, and 1 μL of one of the stem-loop reverse transcription primers. The reaction volume was adjusted to 20 μL with ddH₂O, and then briefly centrifuged before performing the reverse transcription. Table 6 shows the system for the reverse transcription.

TABLE 6
System for the reverse transcription of the target miRNAs
using each of the stem-loop reverse transcription primers

| Reagent | 20 μL system | |
|---|---|---|
| 10X RT MIX | 2 | μL |
| HiScript II Enzyme Mix | 2 | μL |
| RNA synthesis template/water | 10 pg-1 μg | |
| Stem-loop reverse transcription primer(s) (2 μM) | 0.5 | μL |
| ddH₂O | To 20 | μL |

The reverse transcription process is performed according to the thermal cycle in Table 7.

TABLE 7
Thermal cycle for the reverse transcription.

| Cycle | Temperature | Time |
|---|---|---|
| 1 | 25° C. | 5 min |
| 1 | 50° C. | 15 min |
| 1 | 85° C. | 5 min |

The reverse transcription products were immediately subjected to the qPCR reaction or stored at −20° C.

When performing the singleplex reverse transcription, a specific stem-loop reverse transcription primer for the reverse transcription of a particular target miRNA was added to the above reverse transcription system, with a concentration of 2 μM and an amount of 0.5 μL. For the multiplex reverse transcription, all of the stem-loop reverse transcription primers, each of which is selected for the reverse transcription of one of the multiple target miRNAs, should be added to the reverse transcription system, with a concentration of 2 μM and an amount of 0.5 μL for each of the stem-loop reverse transcription primers. The stem-loop sequences used for the stem-loop reverse transcription primers involved in the following experiments are shown in Table 2.

qPCR System and Procedure

Take 2 μL of the reverse transcription products and perform the qPCR quantification according to the following qPCR system and procedure in Tables 8 and 9.

TABLE 8
System for the qPCR of the reverse transcription products

| Reagent | 20 μL system | |
|---|---|---|
| Reverse transcription product | 2 | μL |
| 2X Taq Pro HS U⁺Probe Master Mix | 10 | μL |
| TaqMan probe (10 μM) | 0.2 | μL |
| Forward primer (10 μM) | 0.4 | μL |
| Reverse primer (10 μM) | 0.4 | μL |
| dd H₂O | To 20 | μL |

TABLE 9
Thermal Cycle for the qPCR

| Cycle | Temperature | Time | |
|---|---|---|---|
| 1X | 37° C. | 2 | min |
| | 95° C. | 30 | s |
| 45X | 95° C. | 10 | s |
| | 60° C. | 30 | s |

When the singleplex qPCR for only one particular target miRNA is performed, the forward primer, the reverse primer, and the probe designed for the particular target miRNA is used in the qPCR system to quantify the particular target miRNAs. In contrast, during the multiplex qPCR, a combination forward primers, a combination of reverse primers, and a combination of probes, designed for each and every of the target miRNAs are all included to quantify the multiple target miRNAs simultaneously. The forward primers, reverse primers, and probes involved in the experiments are shown in Tables 3-5.

### Specificity of the Stem-Loop Reverse Transcription Primers Based on the Lengths of the Anchor Sequences

Each of the stem-loop reverse transcription primers selected for one of the target miRNA is formed into three variants; and each of the variants has the selected stem-loop structure linked to an anchor sequence of a different length: 6 nt, 8 nt, 11 nt. Therefore, for each target miRNA, three stem-loop reverse transcription primer variants are formed, and each of the variants was separately used to singleplex reverse transcribe a single target miRNA template, each of which contains one of miR-16-5p, miR-34c-5p, miR-9-3p, and miR-9-5p. The singleplex reverse transcription products were then subjected to singleplex qPCR quantification.

TABLE 10
Stem-loop reverse transcription primers having
anchor sequences of different lengths

| | | Anchor |
|---|---|---|
| Primer Name (SEQ ID No.) | Stem loop structure | bases |
| miR-16-5p-RT11 (SEQ ID No. 62) | Stem loop structure 1 | 11 |
| miR-16-5p-RT8 (SEQ ID No. 5) | (SEQ ID No. 9) | 8 |
| miR-16-5p-RT6 (SEQ ID No. 63) | | 6 |
| miR-34c-5p-RT11 (SEQ ID No. 64) | Stem loop structure 2 | 11 |
| miR-34c-5p-RT8 (SEQ ID No. 65) | (SEQ ID No. 10) | 8 |
| miR-34c-5p-RT6 (SEQ ID No. 6) | | 6 |
| miR-9-3p-5p-RT11 (SEQ ID No. 66) | Stem loop structure 3 | 11 |
| miR-9-3p-5p-RT8 (SEQ ID No. 67) | (SEQ ID No. 11) | 8 |
| miR-9-3p-5p-RT6 (SEQ ID No. 7) | | 6 |
| miR-9-5p-RT11 (SEQ ID No. 8) | Stem loop structure 4 | 11 |
| miR-9-5p-RT8 (SEQ ID No. 68) | (SEQ ID No. 12) | 8 |
| miR-9-5p-RT6 (SEQ ID No. 69) | | 6 |

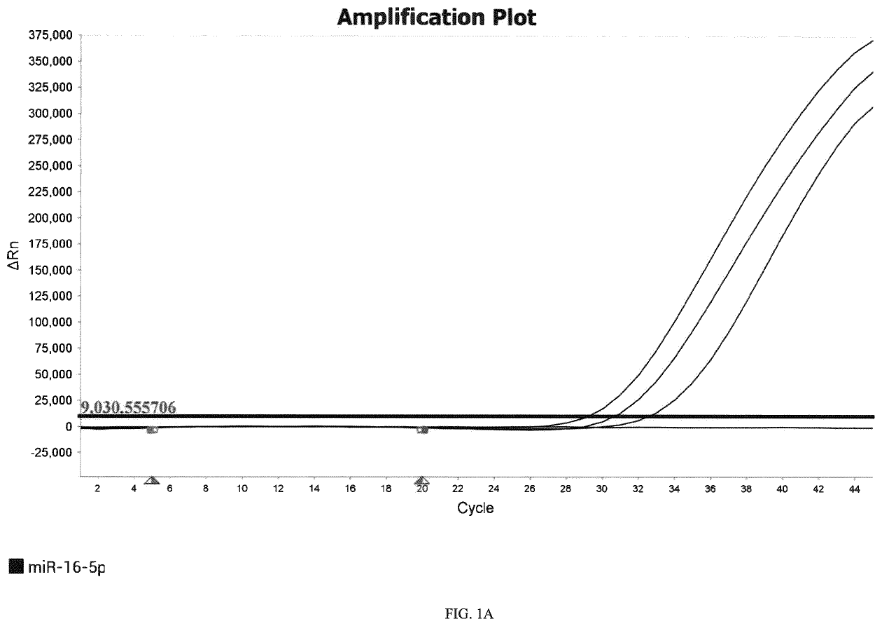

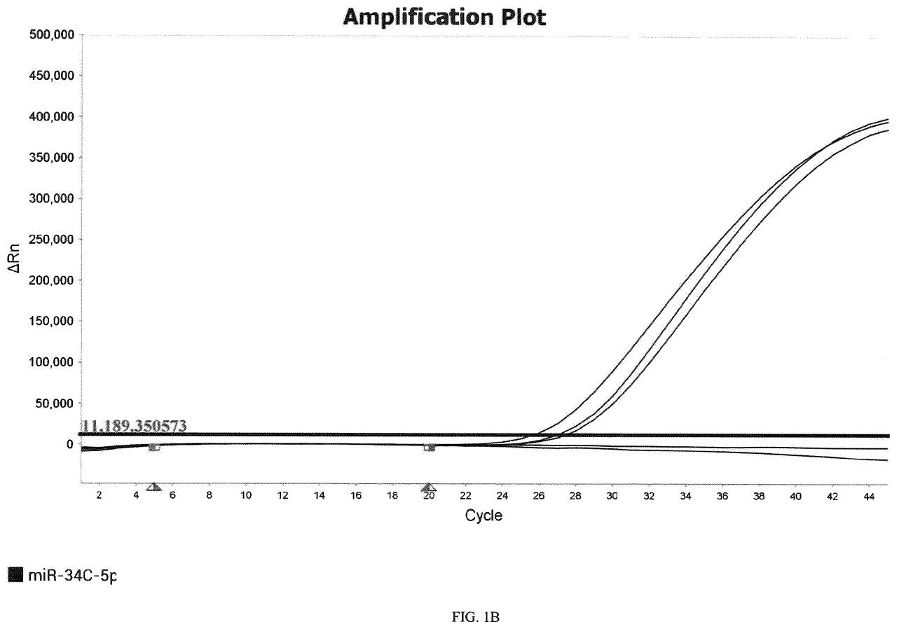

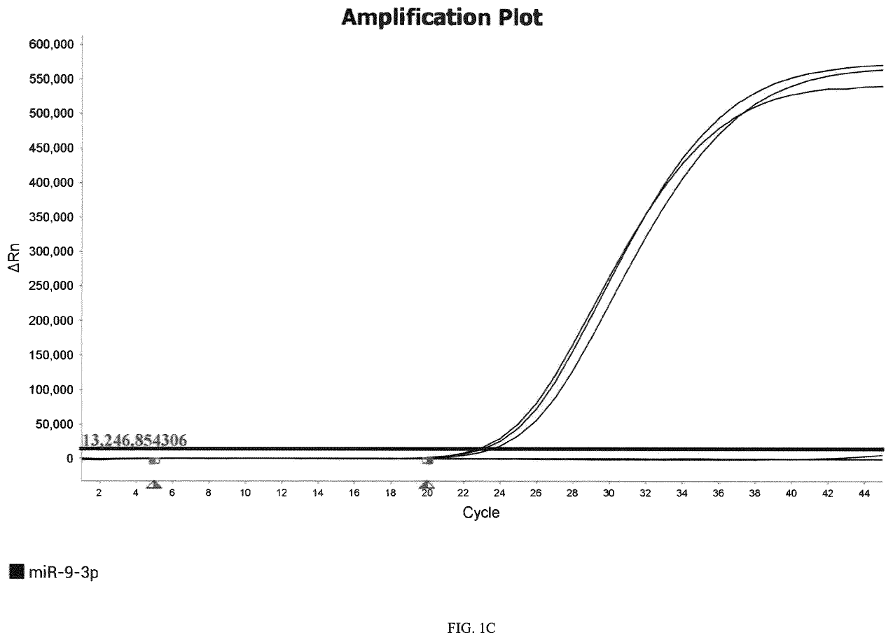

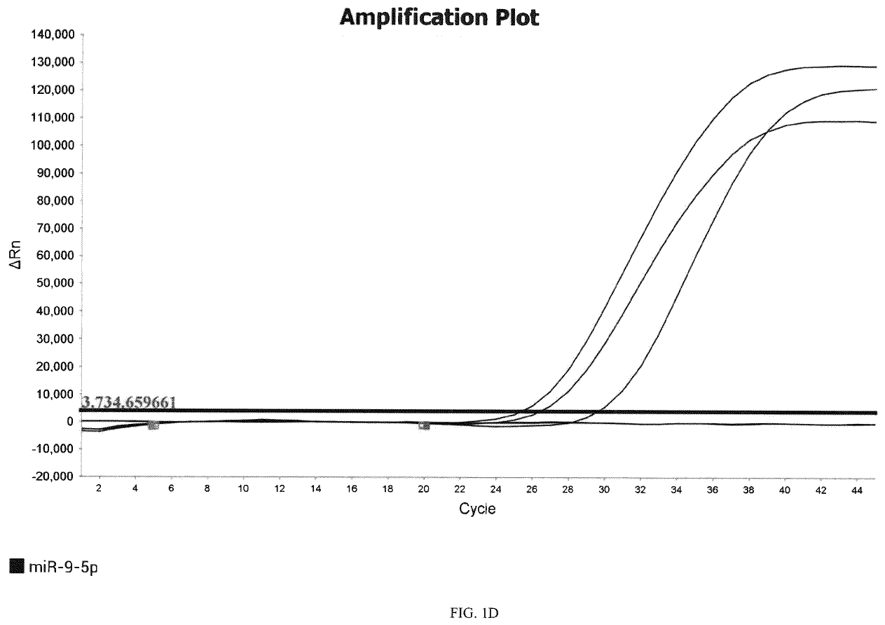

As shown in FIGS. 1A-D, each of the stem-loop reverse transcription primer variants specifically reverse transcribes the target miRNA for which it is designed to reverse transcribe, while each of the stem-loop reverse transcription primer variants demonstrates negative results for the negative control group using ddH2O, indicating good specificity. Results are shown in Table 11.

TABLE 11
Singleplex RT- singleplex qPCR using each of the
stem-loop reverse transcription primer variants

| | Anchor | | | | | |
|---|---|---|---|---|---|---|
| Experiment | sequence | | | | | |
| group | length | | miR-16-5p | miR-34c-5p | miR-9-3p | miR-9-5p |
| Single miRNA | 6 | nt | 29.006 | 25.339 | 24.946 | 22.540 |
| template | 8 | nt | 25.222 | 26.934 | 25.852 | 23.034 |
| | 11 | nt | 25.994 | 26.599 | 26.259 | 23.760 |
| ddH₂O | 6 | nt | Undetermined | Undetermined | Undetermined | Undetermined |
| | 8 | nt | Undetermined | Undetermined | Undetermined | Undetermined |
| | 11 | nt | Undetermined | Undetermined | Undetermined | Undetermined |

| | | | | | |
|---|---|---|---|---|---|
| ddH₂O | H | Undetermined | Undetermined | Undetermined | Undetermined |

### Screening the Anchor Sequence Lengths Achieving the Multi-Specificity for the Combination of Stem-Loop Reverse Transcription Primers

The following paragraphs describe an example in which the multi-specificity of the stem-loop reverse transcription primers is achieved by adjusting and/or screening the length of the anchor sequence of each of the stem-loop reverse transcription primers. During the screening process, the present invention individually accomplishes a duplex stem-loop reverse transcription primer combination and its use in duplex RT-qPCR, a triplex stem-loop reverse transcription primer combination and its use in triplex RT-qPCR, and finally a quadruplex stem-loop reverse transcription primer combination and its use in qaudruplex RT-qPCR. In particular, the duplex stem-loop reverse transcription primer combination includes two stem-loop reverse transcription primers and enables simultaneous quantifications of two of the target miRNAs in the duplex RT-qPCR. The triplex stem-loop reverse transcription primer combination includes three stem-loop reverse transcription primers and enables simultaneous quantifications of three of the target miRNAs in the triplex RT-qPCR. Consequently, a quadruplex stem-loop reverse transcription primer combination including four stem-loop reverse transcription primers is constructed, and effectively quantifies miR-16-5p, miR-34c-5p, miR-9-3p, and miR-9-5p in the quadruplex RT-qPCR. Based on homology restrictions, the screening method used in this example was expanded to other target miRNAs multiplex quantification detections.

Table 10 shows the stem-loop reverse transcription primers used in the experiment, while Tables 3-5 show the forward and reverse primers and probes used in the qPCR process.

In one embodiment of the invention, a multiplex system include at least a multiplex stem-loop reverse transcription primer combination having multiple stem-loop reverse transcription primers, each of which corresponds to one target miRNA and reverse transcribes the one target miRNA in a singleplex or multiplex RT reaction. In one embodiment, the multiplex system also includes multiple forward primers, multiple reverse primers, and multiple probes, each of the multiple forward primers, each of multiple reverse primers, and each of the probes correspond to one target miRNA, and specifically participate in a singleplex or multiplex qPCR of the reverse transcription product of the one target miRNA.

In one embodiment, the multiplex system refers to a duplex system, in which two stem-loop reverse transcription primers are used for quantifying two target miRNAs in a duplex RT-qPCR. In one embodiment, the multiplex system refers to a triplex system, in which three stem-loop reverse transcription primers are used for quantifying three target miRNAs in a triplex RT-qPCR. In one embodiment, the multiplex system refers to a quadruplex system, in which four stem-loop reverse transcription primers are used for quantifying four target miRNAs in a quadruplex RT-qPCR. In one embodiment, a multiplex system comprises more than four stem-loop reverse transcription primers, which are used for quantifying more than four target miRNAs in a multiplex RT-qPCR.

Duplex System for Quantification of Two Target miRNAs

The duplex system includes a stem-loop reverse transcription primer combination includes any two of the stem-loop reverse transcription primers each of which demonstrates specificity to one of the target miRNAs, their corresponding forward/reverse primers, and probes. The duplex RT-qPCR is performed to verify the sensitivity and specificity of the duplex stem-loop reverse transcription primer combination. In one embodiment, two of the target miRNAs are randomly selected to form one or more combinations.

In one embodiment, two combinations are formed:
Target miRNA combination 1: miR-34c-5p and miR-9-3p;
Target miRNA combination 2: miR-34c-5p and miR-16-5p.

For a mixed RNA template having either target miRNAs in combination 1 or combination 2 above, the corresponding stem-loop reverse transcription primers having anchor sequences of different lengths were used to perform the duplex RT with the mixed RNA template, and ddH₂O as the negative control. The duplex RT-qPCR quantification was performed using duplex reverse transcription primers for the mixed RNA template/negative control. Amplification results were observed to quantify the target miRNAs in the mixed RNA template. The specificity of each of the stem-loop reverse transcription primers having an anchor sequence of different lengths was observed. The anchor length for each of the two stem-loop reverse transcription primers in the duplex system is determined according to the specificity observed.

Based on the above steps, it was finally determined that for miR-34c-5p, a stem-loop reverse transcription primer with an anchor sequence length of 6 nt is selected, having the SEQ ID No. 6. For miR-16-5p, a stem-loop reverse transcription primer with an anchor sequence length of 8 nt is selected, having the SEQ ID No. 5. For miR-9-3p, a stem-loop reverse transcription primer with an anchor sequence length of 6 nt is selected, having the SEQ ID No. 7.

### Specificity of the Duplex Stem-Loop Reverse Transcription Primer Combinations

Target miRNA combination 1 of miR-34c-5p and miR-9-3p is used to form a mixed target miRNA template 1, and the target miRNA combination 2 of miR-34c-5p and miR-16-5p is used to form a mixed target miRNA template 2.

Each of the mixed target miRNA templates 1 and 2 separately undergoes a singleplex RT-qPCRs, in which each of the two corresponding stem-loop reverse transcription primers selected for the two target miRNAs is used in each of the singleplex RT-qPCRs.

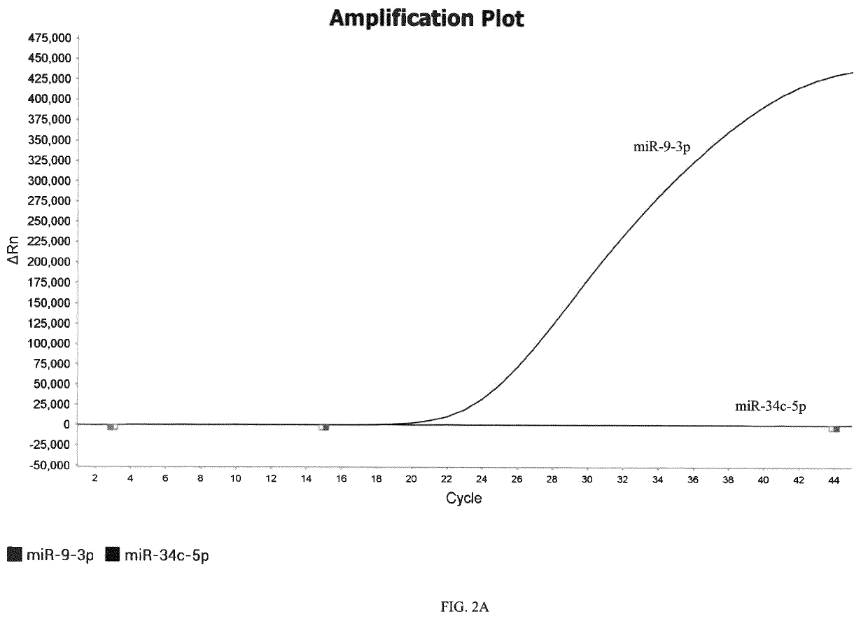

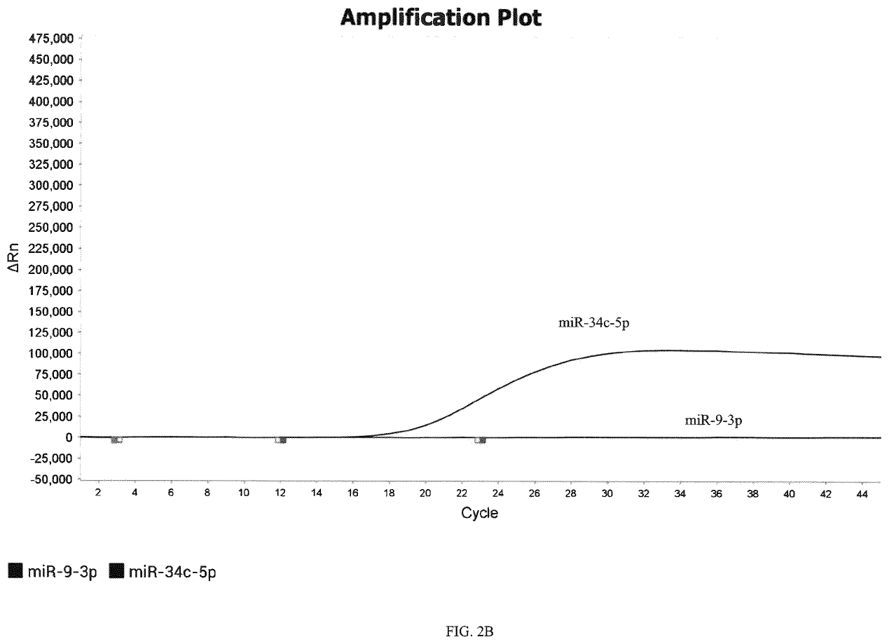

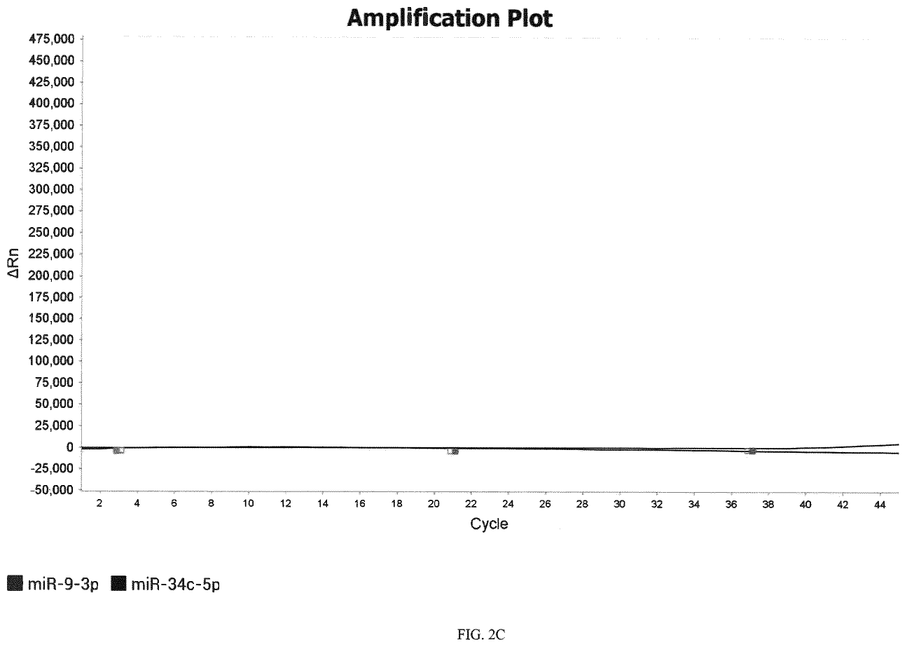

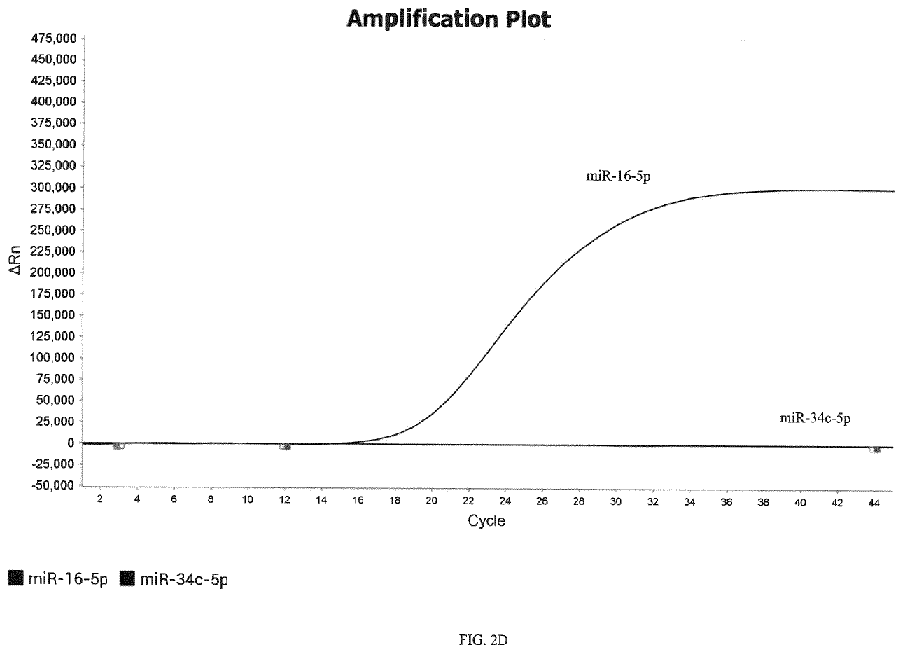

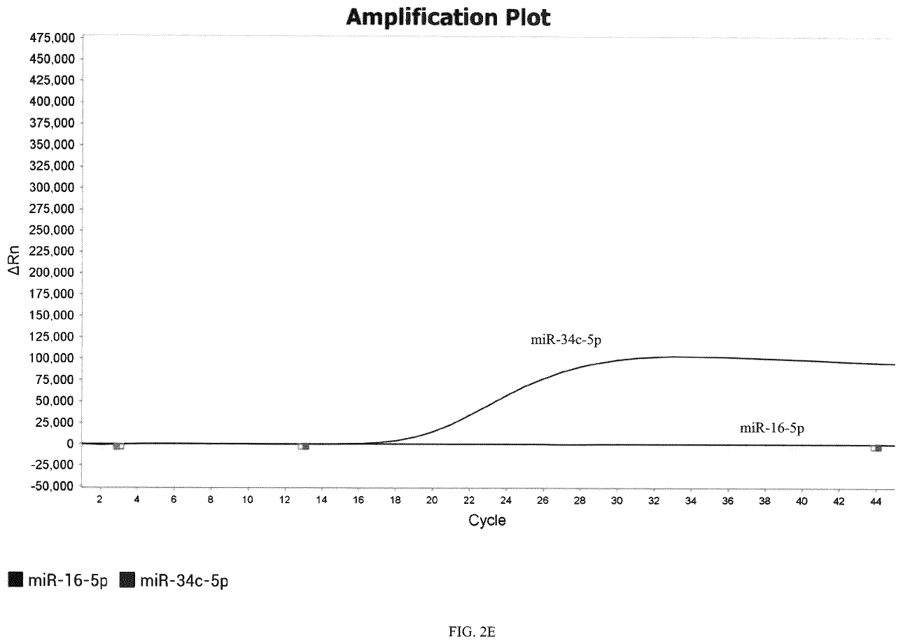

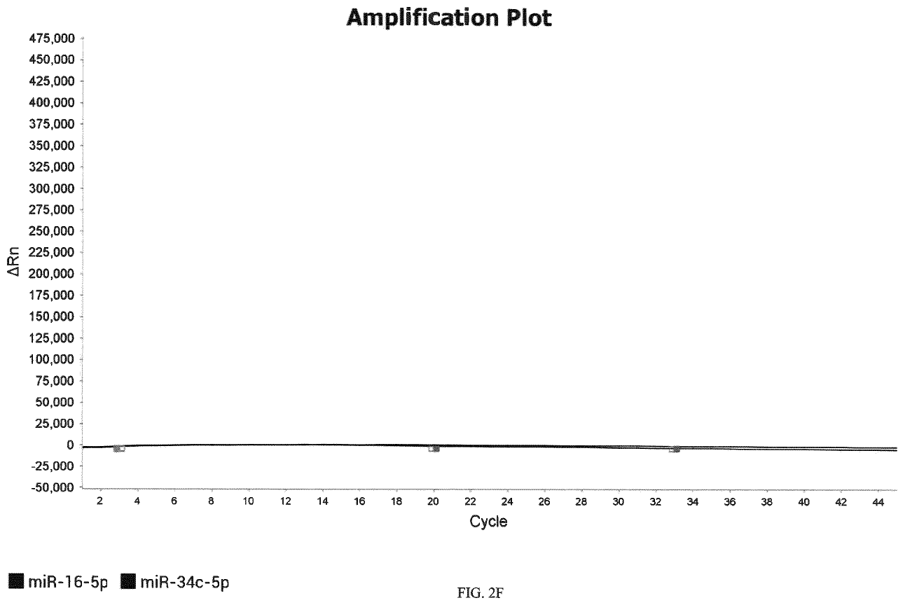

FIGS. 2A-F shows the results of the four singleplex RT-qPCR of two target miRNAs combinations 1 and 2. In particular, it is shown in FIGS. 2A-C that the stem-loop reverse transcription primer for miR-9-3p (SEQ ID No. 7) specifically reverse transcribes miR-9-3p, and the stem-loop reverse transcription primer for miR-34c-5p (SEQ ID No. 6) specifically reverse transcribes miR-34c-5p. Both of the stem-loop reverse transcription primers shows no signal in the negative control group using ddH₂O, as shown in FIG. 2C. It is shown in FIGS. 2D-F that the stem-loop reverse transcription primer for miR-16-5p (SEQ ID No. 5) specifically reverse transcribes miR-16-5p, and the stem-loop reverse transcription primer for miR-34c-5p (SEQ ID No. 6) specifically reverse transcribes miR-34c-5p. Both of the stem-loop reverse transcription primers shows no signal in the negative control group using ddH₂O, as shown in FIG. 2F.

Once the singleplex RT-qPCRs show good specificity for each of the selected corresponding stem-loop reverse transcription primers, the duplex RT-qPCR is performed for determination if there is any undesired cross-reaction between the two corresponding stem-loop reverse transcription primers selected for the target miRNA combinations 1 and 2.

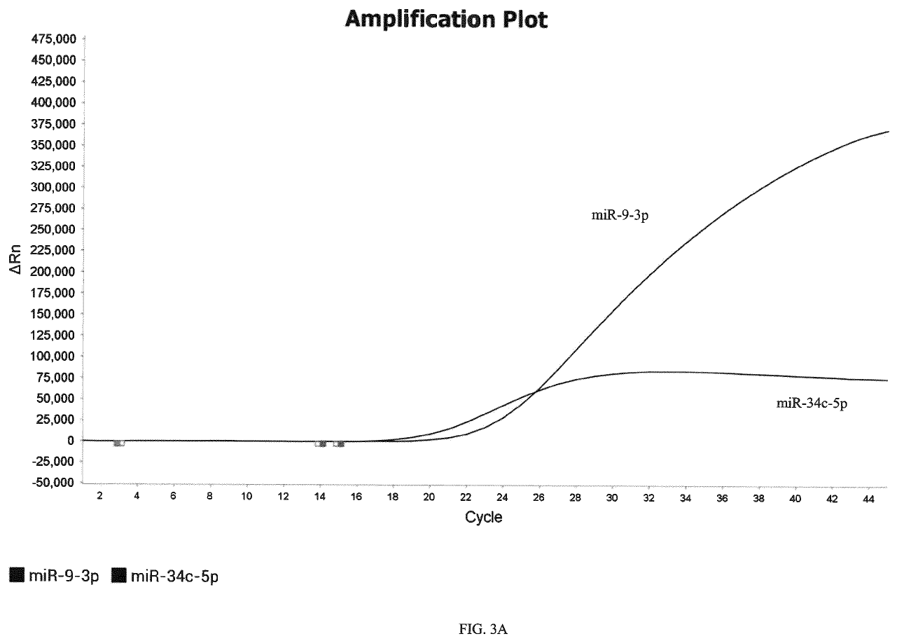

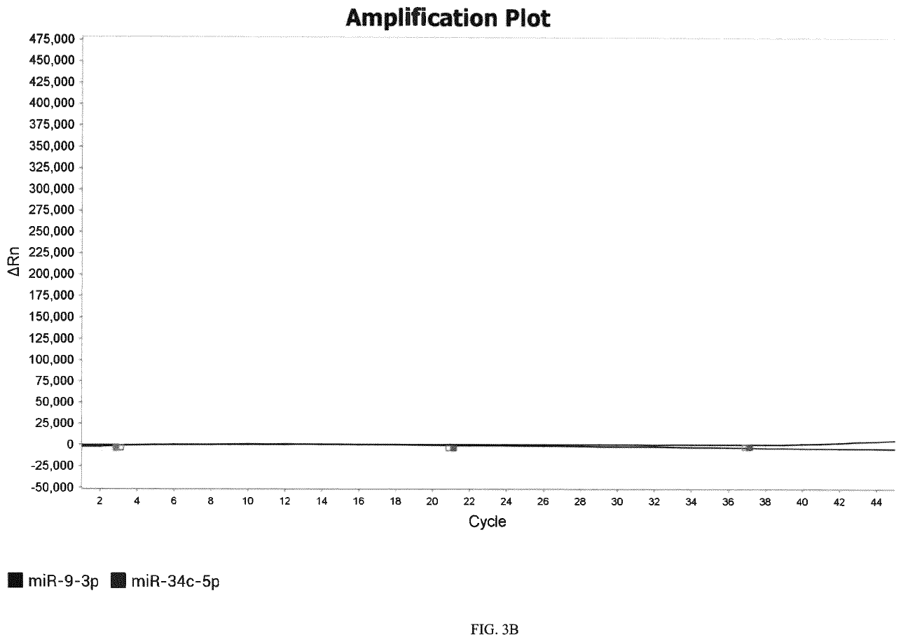

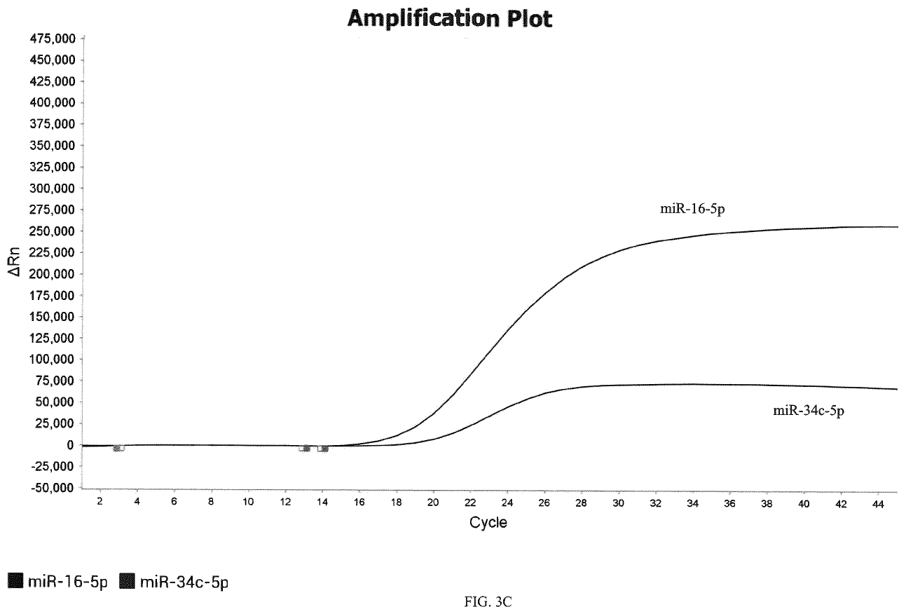

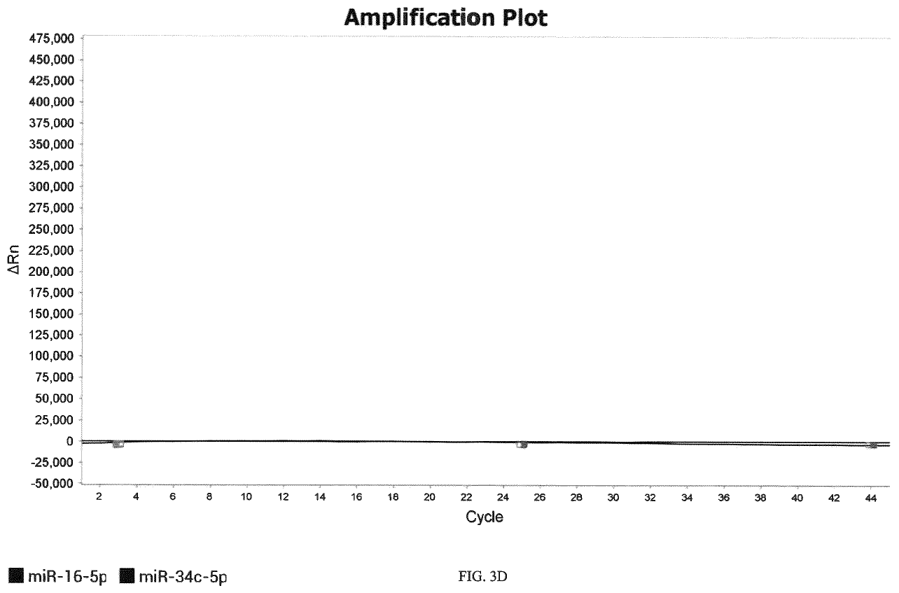

Each of the mixed target miRNA templates 1 and 2 separately undergoes the duplex RT-qPCR using the duplex stem-loop reverse transcription primer combination including the two corresponding stem-loop reverse transcription primers selected for the target miRNA combinations 1 and 2. FIGS. 3A-D shows the results of the two duplex RT-qPCRs of the two target miRNAs combinations. FIGS. 3A and 3C shows normal amplification curves for the target miRNAs, and FIGS. 3B and 3D shows no signal in the negative control group using ddH₂O. This indicates that there exists no cross-reaction between the stem-loop reverse transcription primer for miR-9-3p (SEQ ID No. 7) and the stem-loop reverse transcription primer for miR-34c-5p (SEQ ID No. 6); and between the stem-loop reverse transcription primer for miR-16-5p (SEQ ID No. 5) and the stem-loop reverse transcription primer for miR-34c-5p (SEQ ID No. 6).

### Sensitivity Test for the Duplex System

The two mixed RNA templates 1 and 2 were used for two groups of experiments targeting miR-34c-5p/miR-9-3p and miR-34c-5p/miR-16-5p, respectively. In particular, the duplex RT-qPCR was performed to determine the lowest detection limit, i.e. sensitivity, for each of the target miRNAs. Specifically, 1 pg of mixed target miRNA templates was added, the duplex reverse transcription was performed, and the resulting product was diluted 10-fold in a gradient to a single copy number for the reverse transcription product.

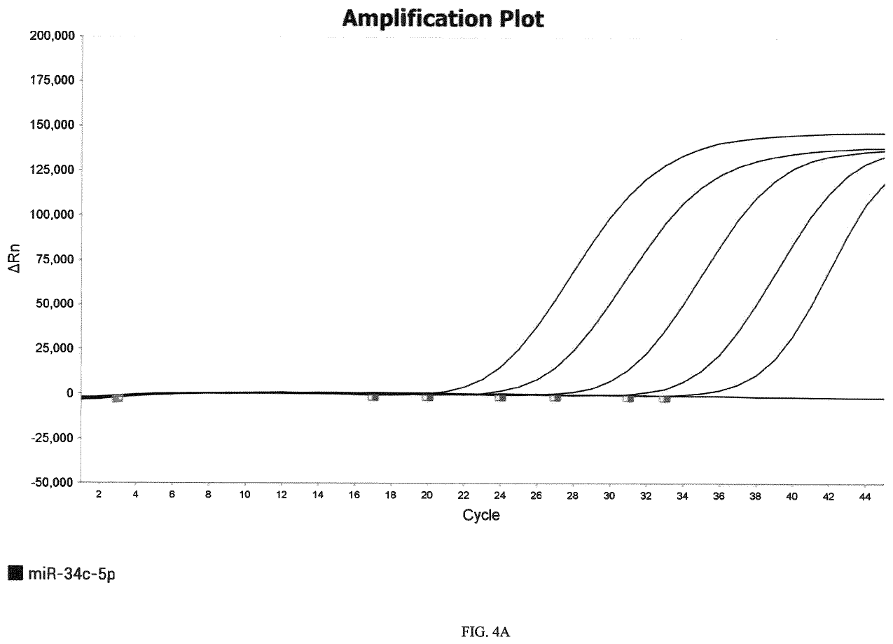

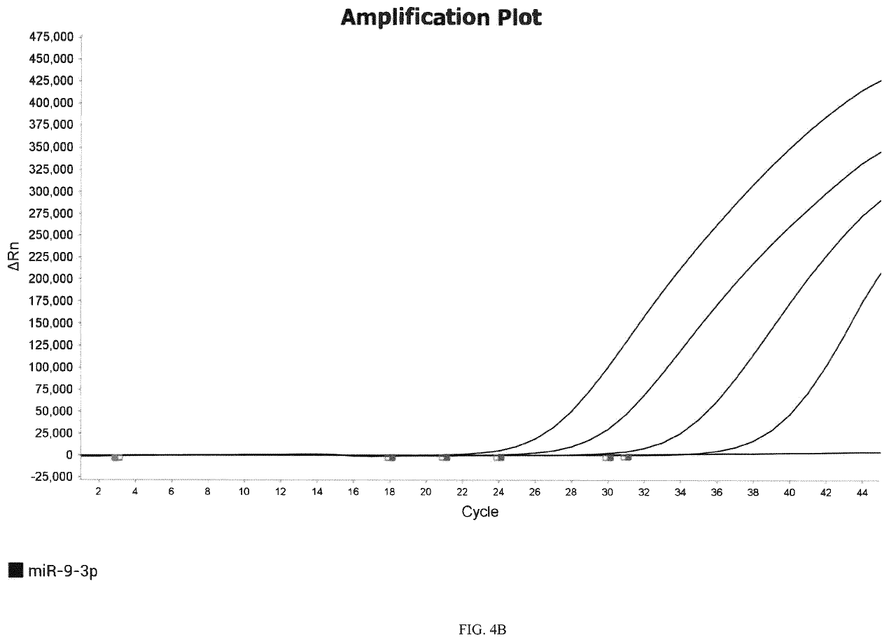

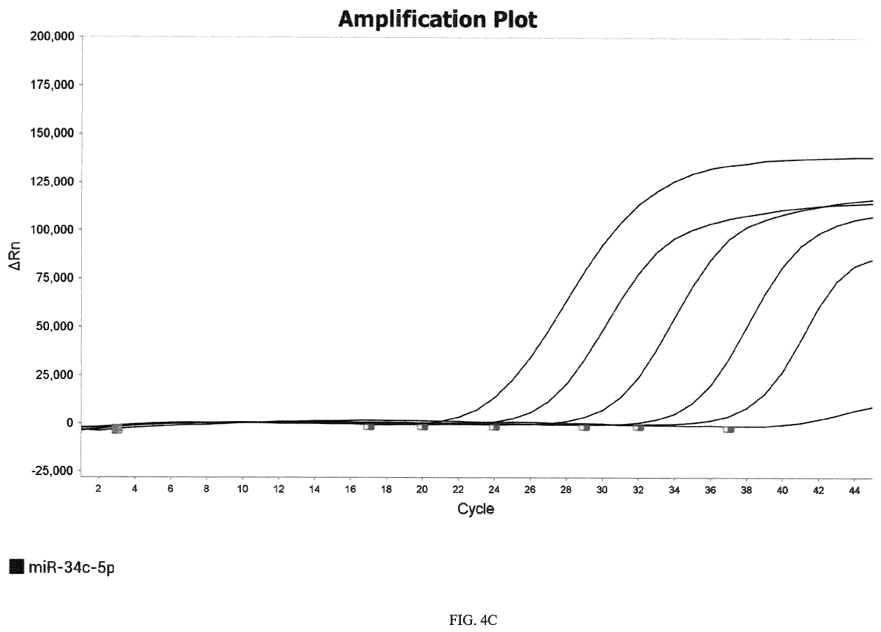

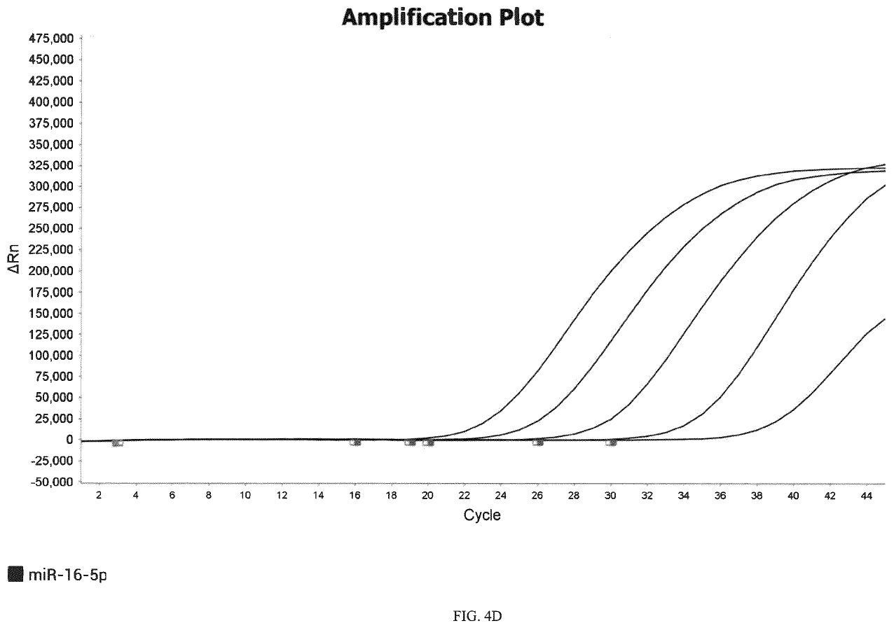

Then, all reverse transcription products obtained were subjected to the duplex qPCR. The results are shown in FIGS. 4A-D (note: the amplification curves in each figure from left to right are the results at concentrations of 1 pg/μL, 0.1 pg/μL, 0.01 pg/μL, 1 fg/μL, and 0.1 fg/μL, respectively).

The results showed that in the combination of miR-34c-5p and miR-9-3p, the detection limit of miR-34c-5p was 0.1 fg/μL, and the detection limit of miR-9-3p was 1 fg/μL. In the combination of miR-34c-5p and miR-16-5p, the detection limit of miR-34c-5p was 0.1 fg/μL, and the detection limit of miR-16-5p was 0.1 fg/μL.

Comparison of the Singleplex and the Duplex Quantification of the Target miRNAs

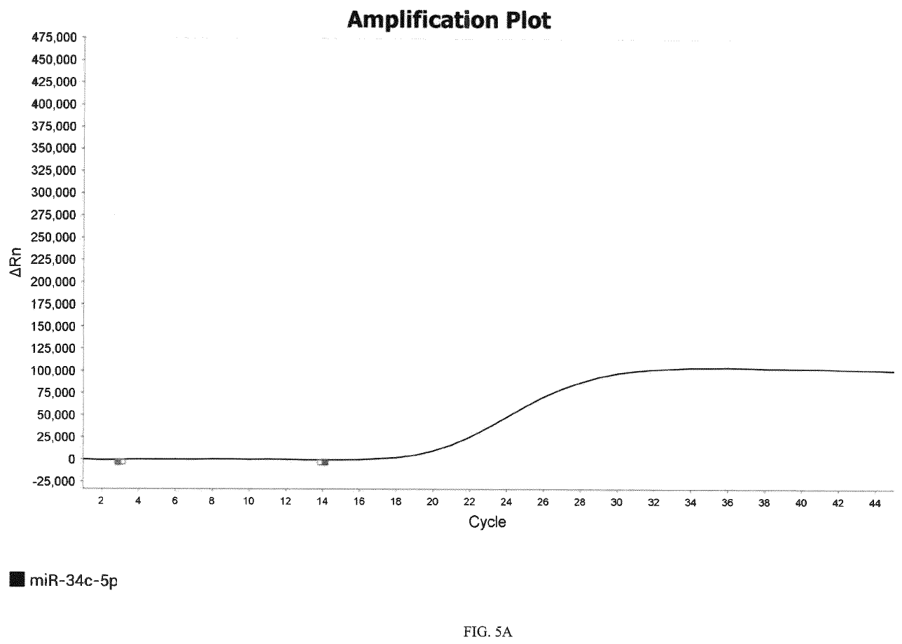

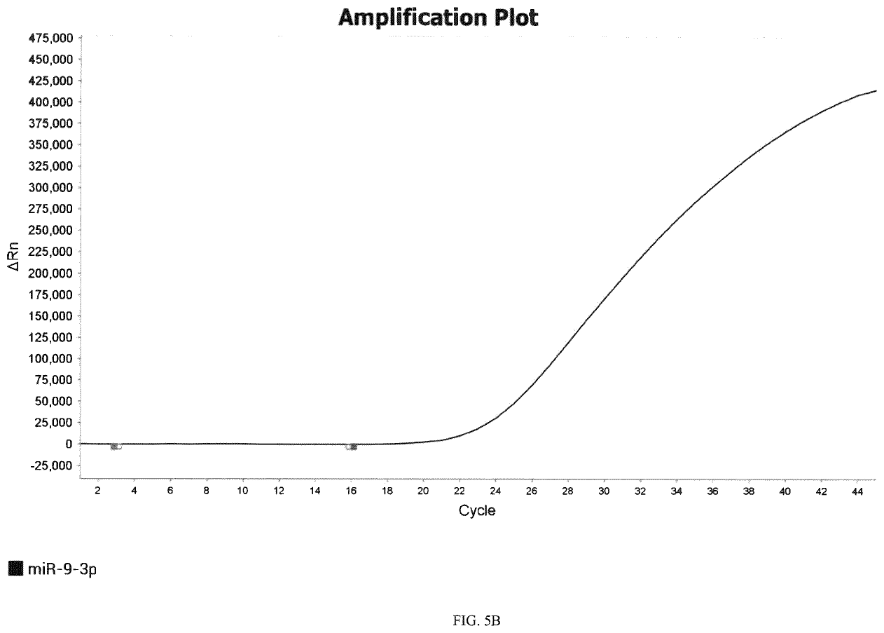

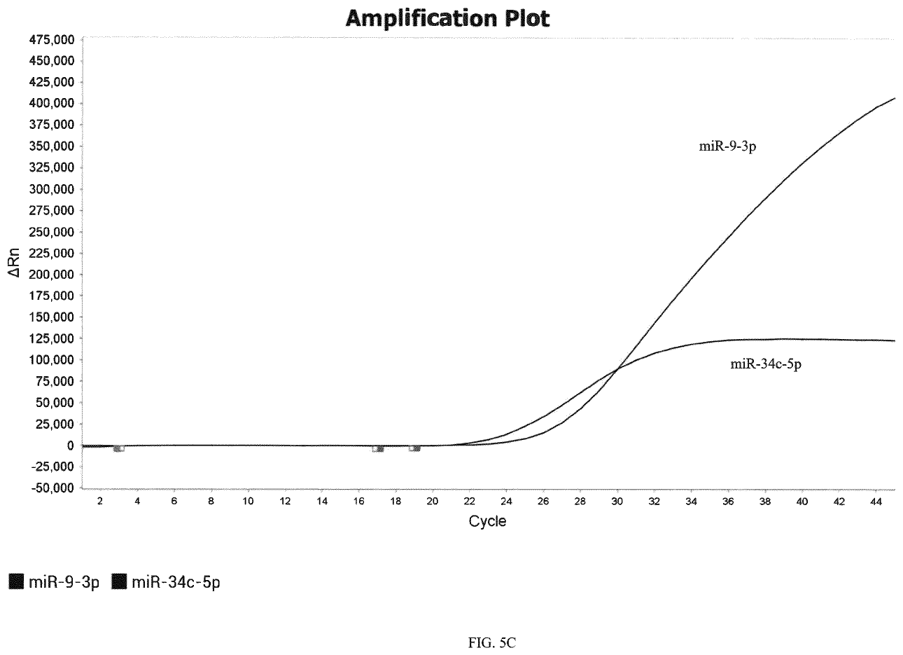

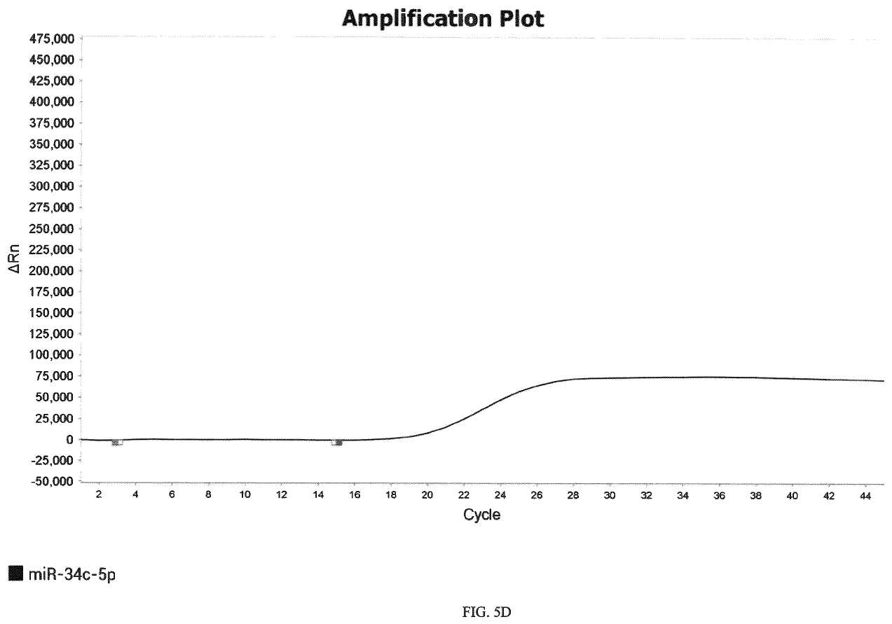

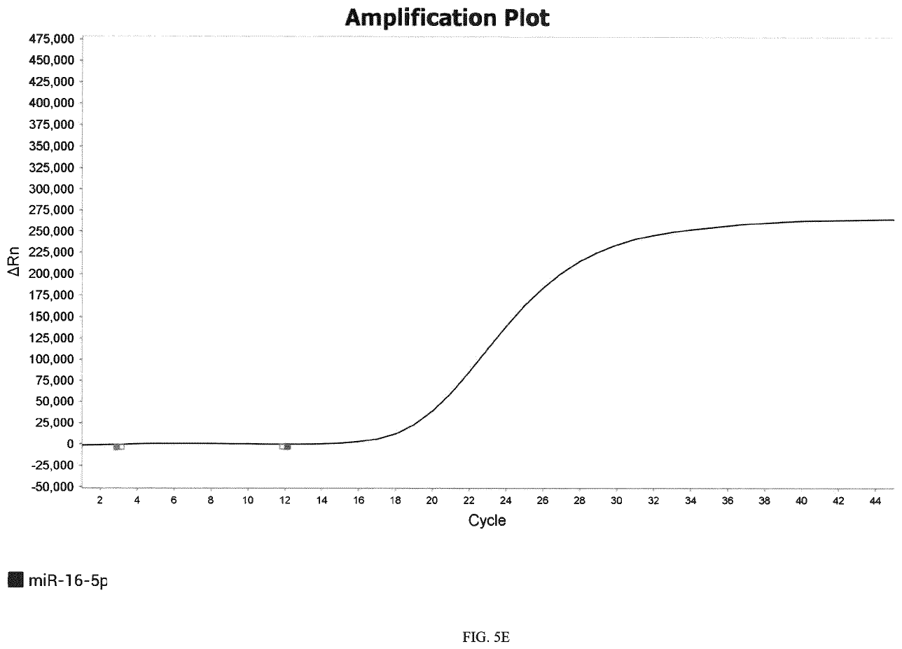

The serum samples were collected for the singleplex and the duplex RT-qPCR quantifications of the target miRNA samples, and the verification of the duplex RT-qPCR was carried out using two target miRNAs combinations: miR-34c-5p and miR-9-3p as combination 1, miR-34c-5p and miR-16-5p as combination 2. The stem-loop reverse transcription primer having an 8 nt anchor sequence (SEQ ID No. 5) is selected for miR-16-5p, and its corresponding probe is labeled with VIC. The stem-loop reverse transcription primer having a 6 nt anchor sequence (SEQ ID No. 7) is selected for miR-9-3p, and its corresponding probe is labeled with ROX. The stem-loop reverse transcription primer having a 6 nt anchor sequence (SEQ ID No. 6) is selected for miR-34c-5p, and its corresponding probe is labeled with CY5. The results are shown in FIGS. 5A-F and Table 12. The results showed that there was no significant difference in amplification curve and Ct value between the singleplex and duplex quantifications of the target miRNAs.

TABLE 12
Singleplex and duplex quantifications of the target miRNAs

| Group | Group I | | Group II | |
|---|---|---|---|---|
| Target miRNAs | miR-34c-5p | miR-9-3p | miR-34c-5p | miR-16-5p |
| Ct value of the | 18.68 | 22.36 | 18.29 | 20.55 |
| singleplex | | | | |
| Ct value of the | 18.85 | 22.56 | 18.47 | 20.78 |
| duplex | | | | |

Triplex System for Quantification of Triple Target miRNAs

The triplex system includes the stem-loop reverse transcription primer combination includes any three of the stem-loop reverse transcription primers each of which demonstrates specificity to one of the target miRNAs, their corresponding forward/reverse primers, and their corresponding probes. Triplex RT-qPCR is performed to verify the sensitivity and specificity of the triplex stem-loop reverse transcription primer combination. In one embodiment, three of the target miRNAs are randomly selected to form one or more combinations.

In one embodiment, two combinations are formed:
Target miRNA combination 3: miR-34c-5p, miR-9-3p, and miR-16-5p;
Target miRNA combination 4: miR-34c-5p, miR-16-5p, and miR-9-5p.

For the mixed RNA template having either the target miRNAs in combination 3 or combination 4 above, the corresponding stem-loop reverse transcription primers having anchor sequences of different lengths were used to perform triplex RT with the mixed RNA template, and ddH₂O as the negative control. Amplification results were observed to quantify the target miRNAs in the mixed RNA templates. The specificity of each of the stem-loop reverse transcription primers having anchor sequences of different lengths was observed. The anchor sequence length for each of the three stem-loop reverse transcription primers in the triplex system is determined according to the specificity observed.

Based on the above steps, it was finally determined that, in the target miRNA combination 3, for miR-34c-5p, the stem-loop reverse transcription primer with the anchor sequence of 6 nt is selected, having the SEQ ID No. 6. For miR-16-5p, the stem-loop reverse transcription primer with the anchor sequence of 8 nt is selected, having the SEQ ID No. 5. For miR-9-3p, the stem-loop reverse transcription primer with the anchor sequence of 6 nt is selected, having the SEQ ID No. 7.

In the target miRNA combination 4, for miR-34c-5p, the stem-loop reverse transcription primer with the anchor sequence of 6 nt is selected, having the SEQ ID No. 6. For miR-16-5p, the stem-loop reverse transcription primer with the anchor sequence of 8 nt is selected, having the SEQ ID No. 5. For miR-9-5p, the stem-loop reverse transcription primer with the anchor sequence of 11 nt is selected, having the SEQ ID No. 8.

### Specificity of the Triplex Stem-Loop Reverse Transcription Primer Combinations

Each of the target miRNAs combination 3 and 4 is used to form mixed target miRNA templates 3 and 4, respectively.

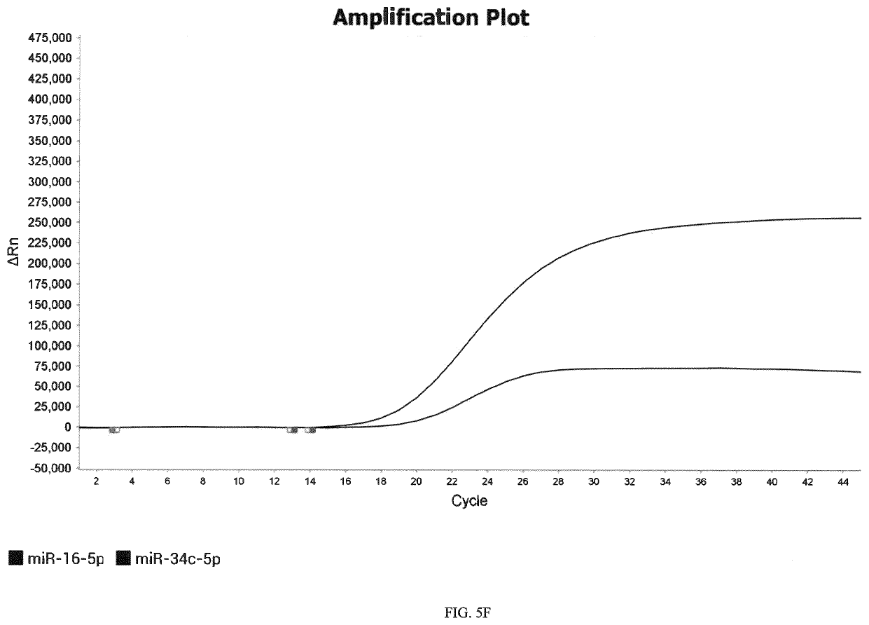

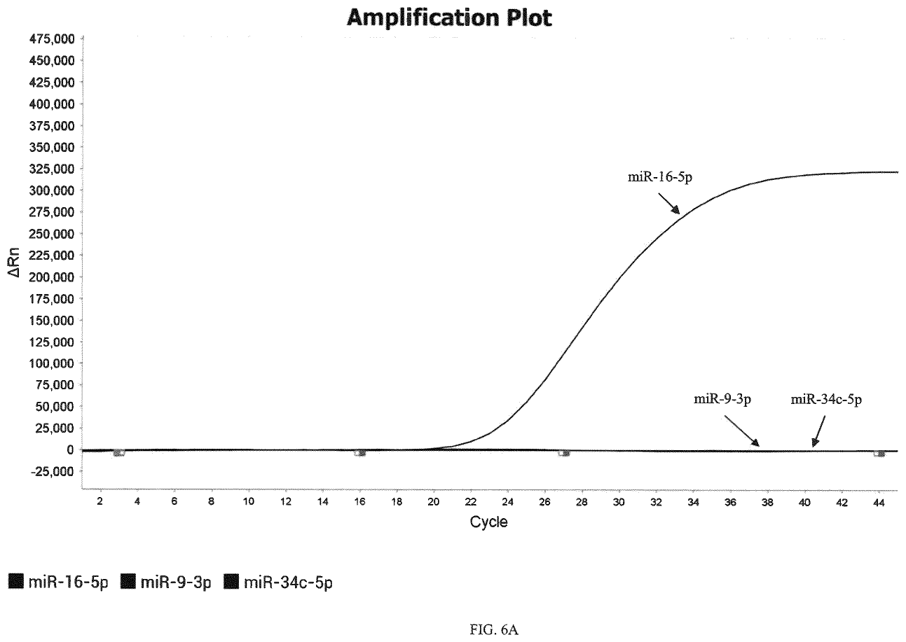

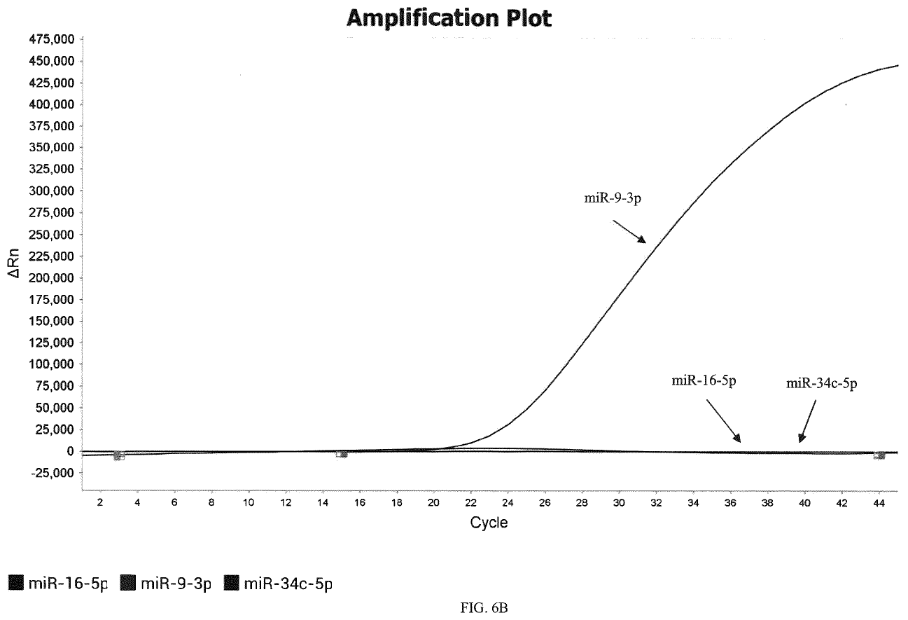

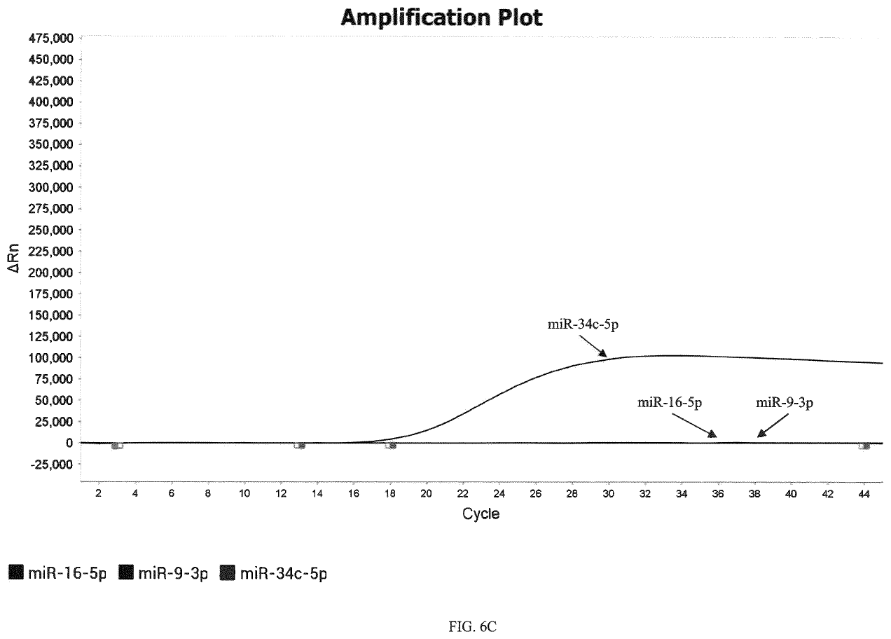

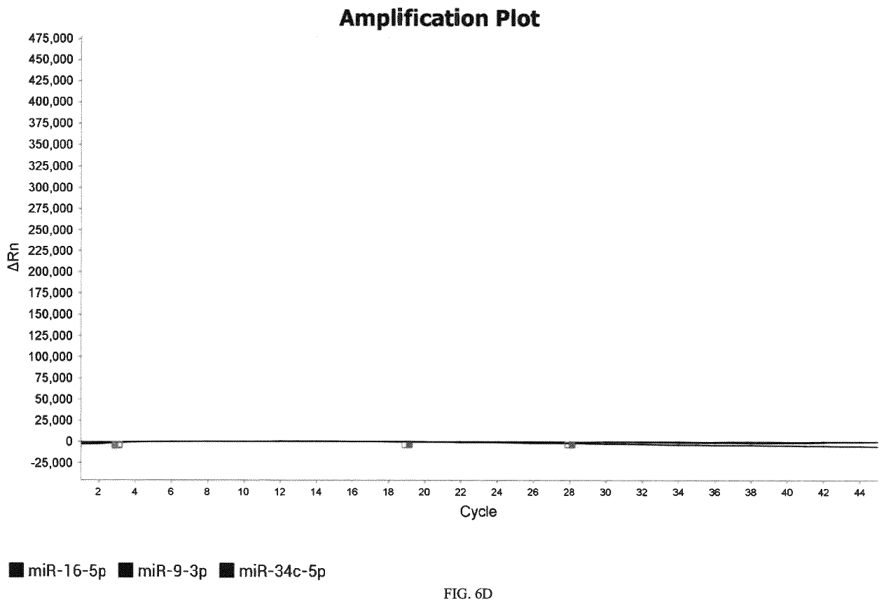

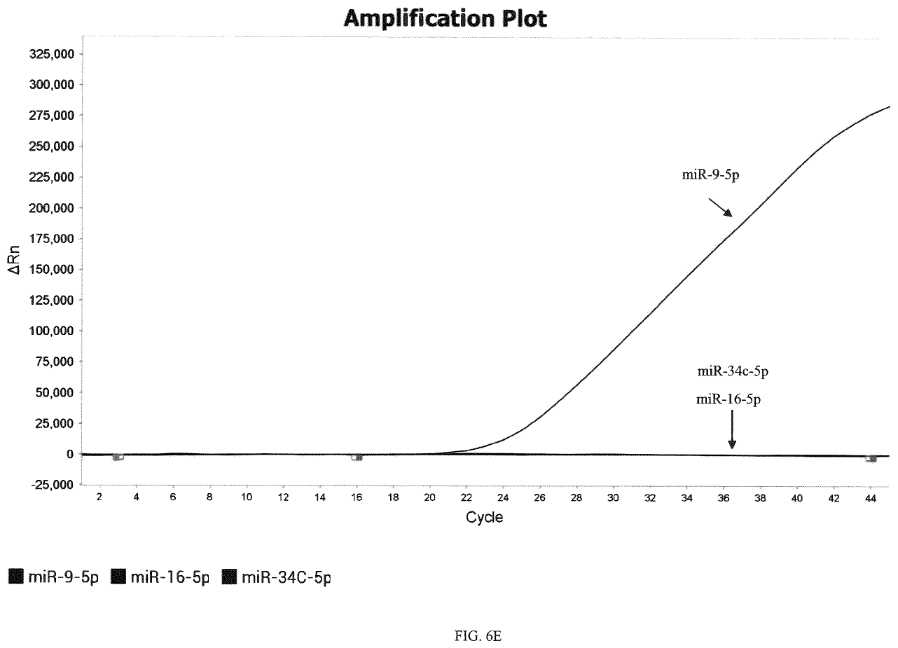

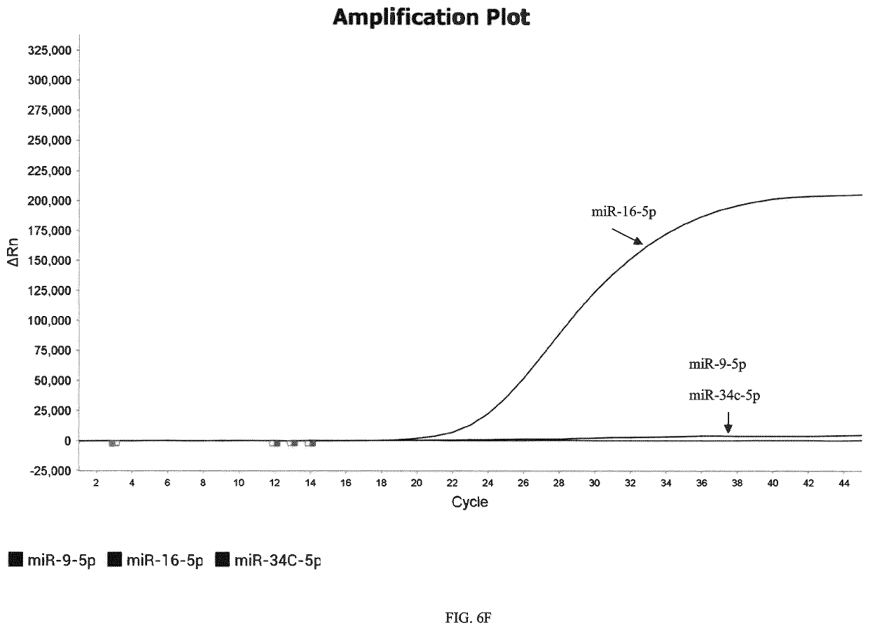

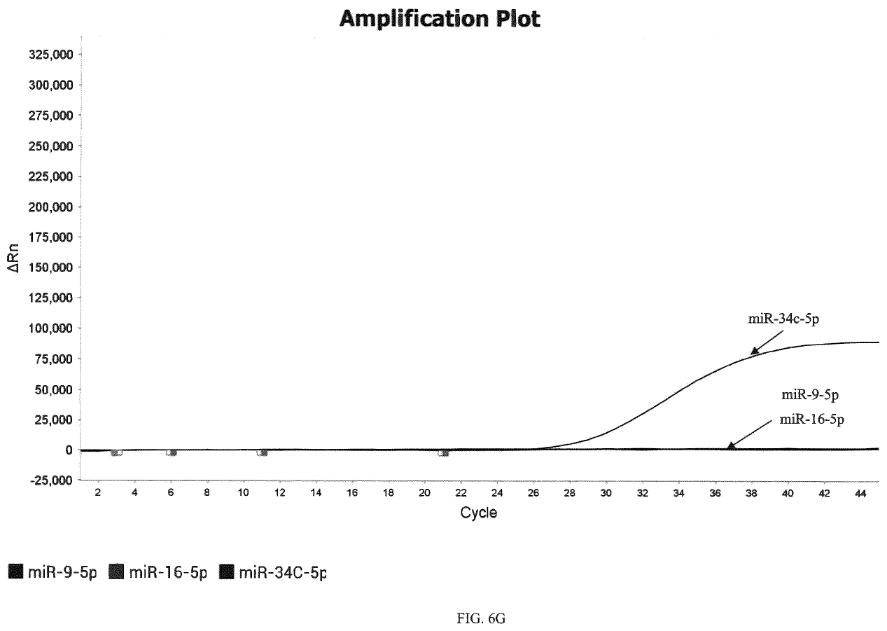

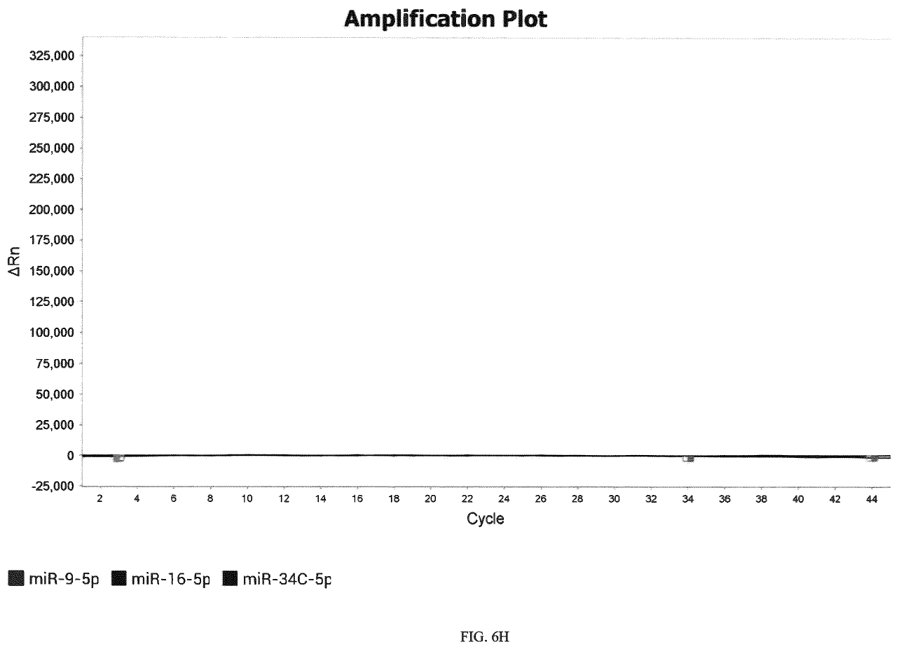

Each of the mixed target miRNA templates 3 and 4 separately undergoes the singleplex RT using each of the three corresponding stem-loop reverse transcription primers selected for the three target miRNAs in each combination. After the singleplex RT, the reverse transcription products from each of the processes of singleplex RT undergo the multiplex qPCR. FIGS. 6A-H shows the results of the six singleplex RT-multiplex qPCRs of two target miRNAs combinations 3 and 4. In particular, it is shown in FIGS. 6A-C that the stem-loop reverse transcription primer for miR-9-3p (SEQ ID No. 7) specifically reverse transcribes miR-9-3p, the stem-loop reverse transcription primer for miR-34c-5p (SEQ ID No. 6) specifically reverse transcribes miR-34c-5p, and the stem-loop reverse transcription primer for miR-16-5p (SEQ ID No. 5) specifically reverse transcribes miR-16-5p. All three of the stem-loop reverse transcription primers shows no signal in the negative control group using ddH₂O in FIG. 6D.

It is shown in FIGS. 6E-G that the stem-loop reverse transcription primer for miR-16-5p (SEQ ID No. 5) specifically reverse transcribes miR-16-5p, the stem-loop reverse transcription primer for miR-34c-5p (SEQ ID No. 6) specifically reverse transcribes miR-34c-5p, and the stem-loop reverse transcription primer for miR-9-5p (SEQ ID No. 8) specifically reverse transcribes miR-9-5p. All three of the stem-loop reverse transcription primers show no signal in the negative control group using ddH₂O, in FIG. 6H.

Once the singleplex RT-multiplex qPCRs show good specificity for each of the selected corresponding stem-loop reverse transcription primers, the triplex RT-qPCR is performed for determining if there is any undesired cross-reaction between the three corresponding stem-loop reverse transcriptions primers selected for the target miRNAs combination 3 and 4.

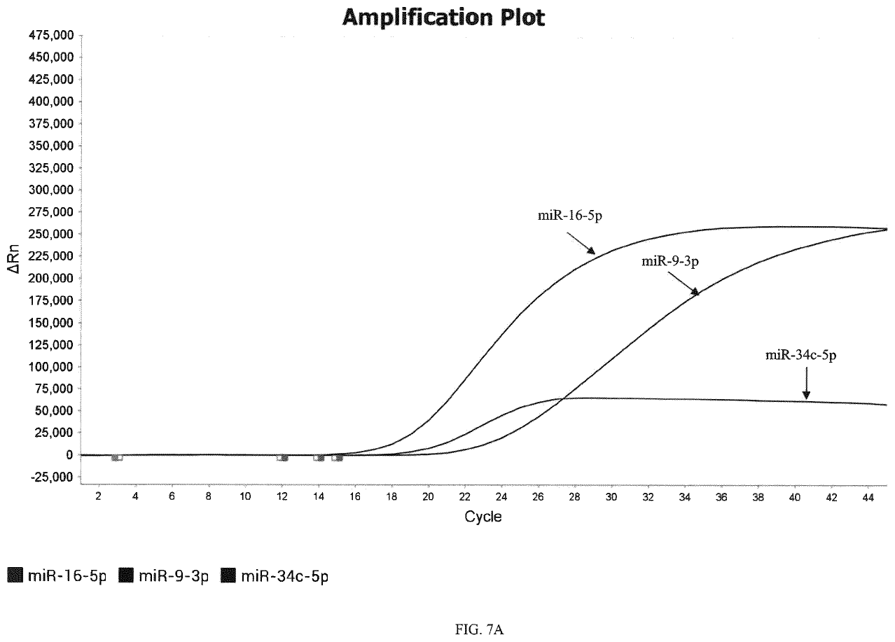

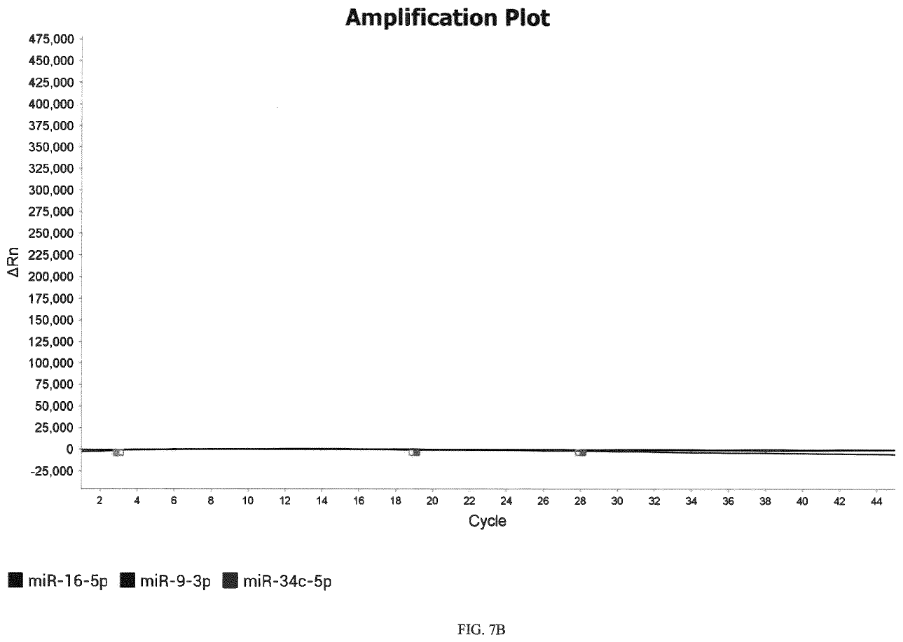

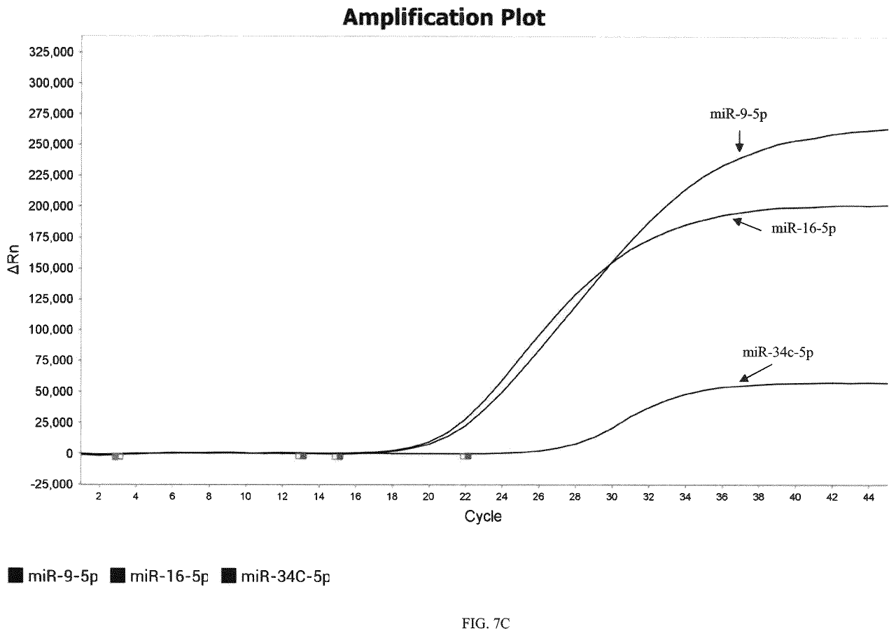

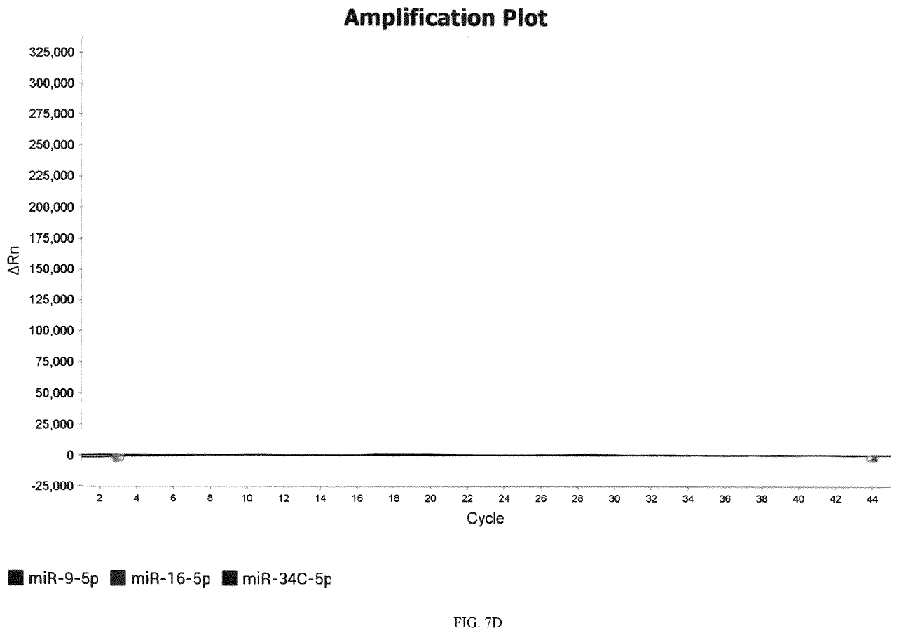

Each of the formed mixed target miRNA templates 3 and 4 separately undergoes the triplex RT-qPCR using its corresponding triplex stem-loop reverse transcription primer combination. FIGS. 7A-D show the results of the two triplex RT-qPCRs of two target miRNAs combinations 3 and 4, with normal amplification curves for the target miRNAs in FIGS. 7A and 7C and no signal in the negative control group using ddH₂O, as shown in FIGS. 7B and 7D. This indicates that there exists no cross-reaction between the stem-loop reverse transcription primer for miR-9-3p (SEQ ID No. 7), the stem-loop reverse transcription primer for miR-34c-5p (SEQ ID No. 6), and the stem-loop reverse transcription primer for miR-16-5p (SEQ ID No. 5); and no cross-reaction between the stem-loop reverse transcription primer for miR-16-5p (SEQ ID No. 5), the stem-loop reverse transcription primer for miR-34c-5p (SEQ ID No. 6), and the stem-loop reverse transcription primer for miR-9-5p (SEQ ID No. 8).

### Sensitivity Test for the Duplex System

The mixed RNA templates having two target miRNA combinations 3 and 4 were used for two groups of experiments, respectively. The triplex RT-qPCR was performed to determine the lowest detection limit, i.e. sensitivity, for each of the target miRNAs. Specifically, 1 pg of mixed target miRNA templates was added, the triplex reverse transcription was performed, and the resulting product was diluted 10-fold in a gradient to a single copy number for the reverse transcription product.

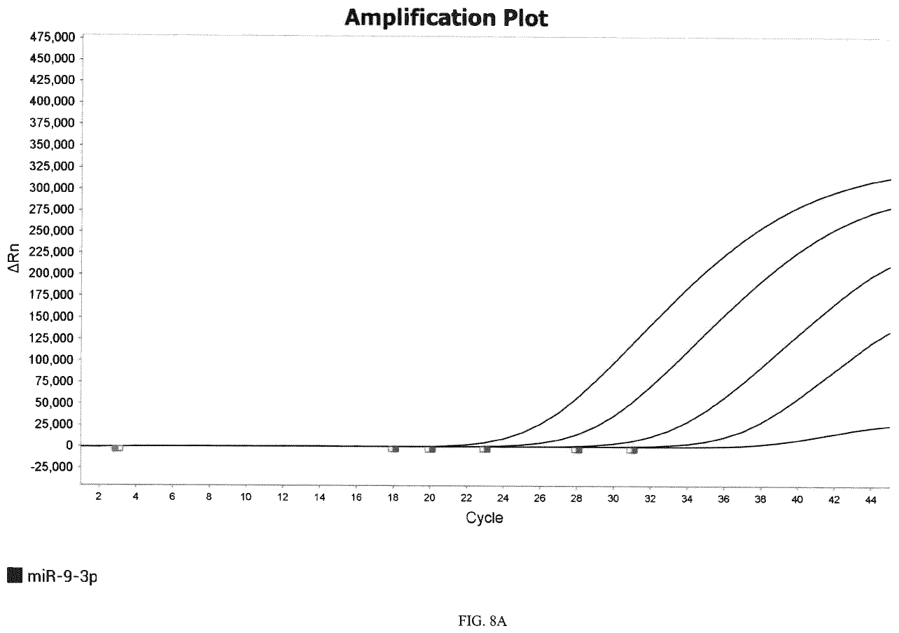

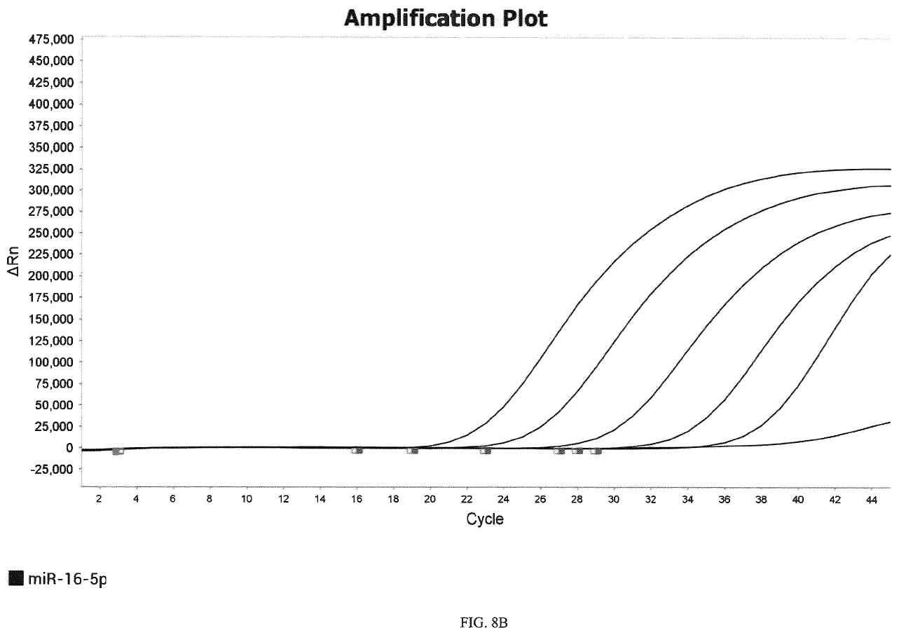

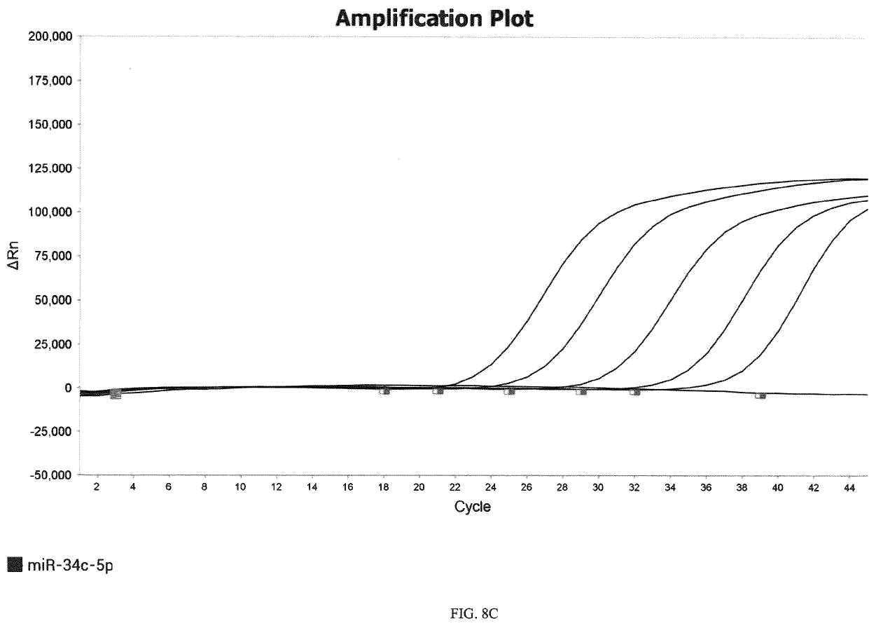

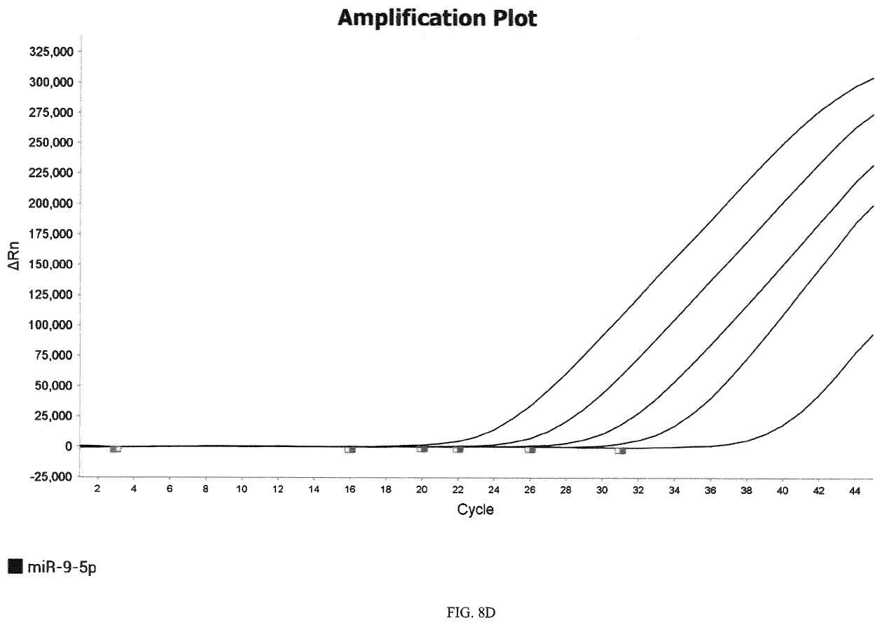

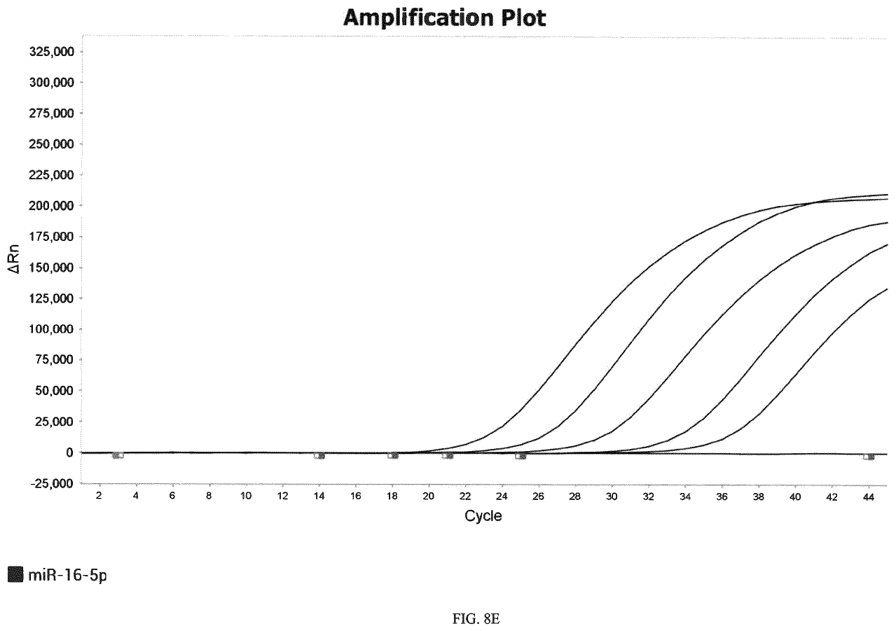

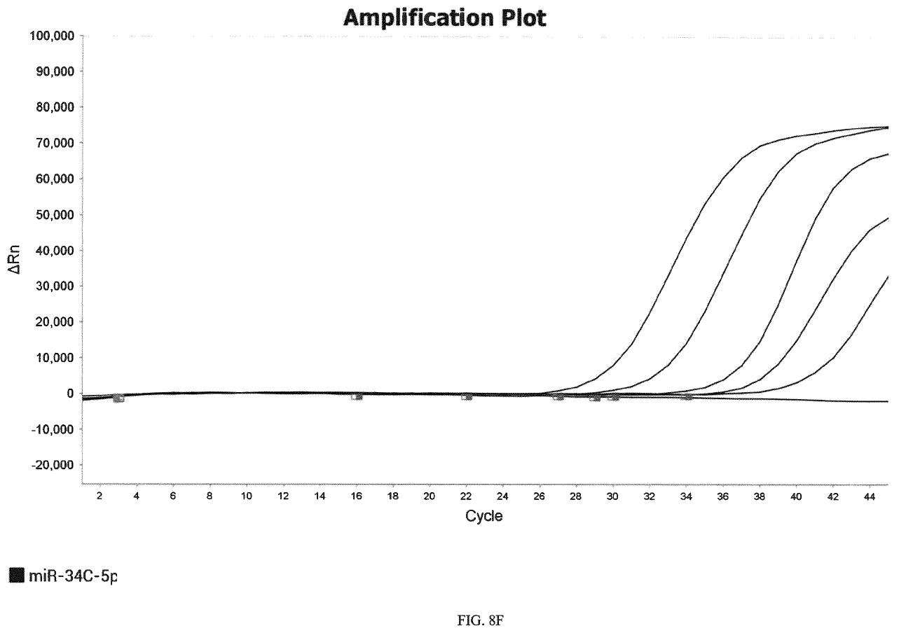

Then, all reverse transcription products obtained were subjected to the triplex qPCR. The results are shown in FIG. 8A-F (note: the amplification curves in each figure from left to right are the results at concentrations of 1 pg/μL, 0.1 pg/μL, 0.01 pg/μL, 1 fg/μL, and 0.1 fg/μL, respectively).

The results showed that in the combination 3, the detection limit of miR-16-5p was 0.1 fg/μL, the detection limit of miR-34c-5p was 0.1 fg/μL, and the detection limit of miR-9-3p was 1 fg/μL. In the combination 4, the detection limit of miR-9-5p was 0.1 fg/μL, the detection limit of miR-34c-5p was 0.1 fg/μL, and the detection limit of miR-16-5p was 0.1 fg/μL.

Comparison of the Singleplex and Triplex Quantification of Target miRNAs

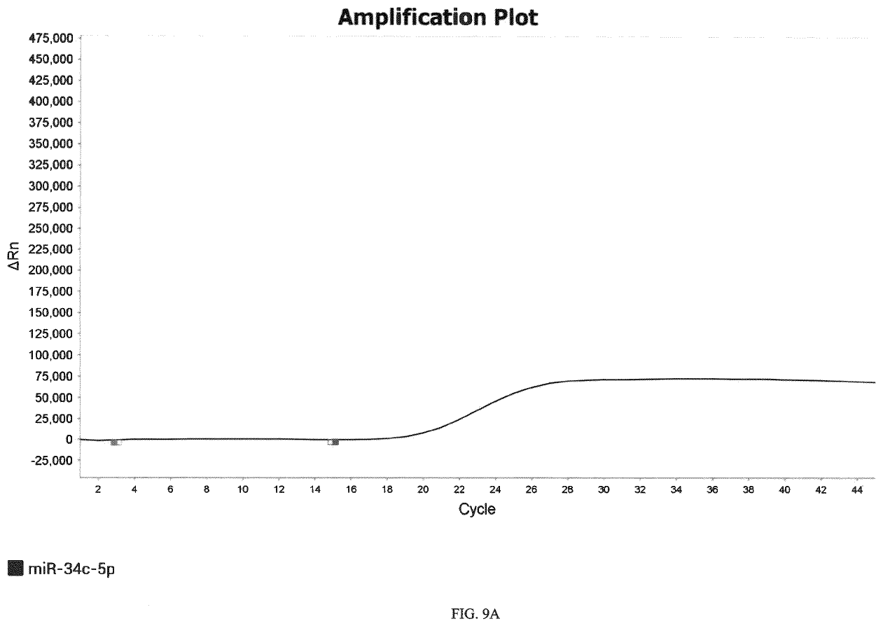

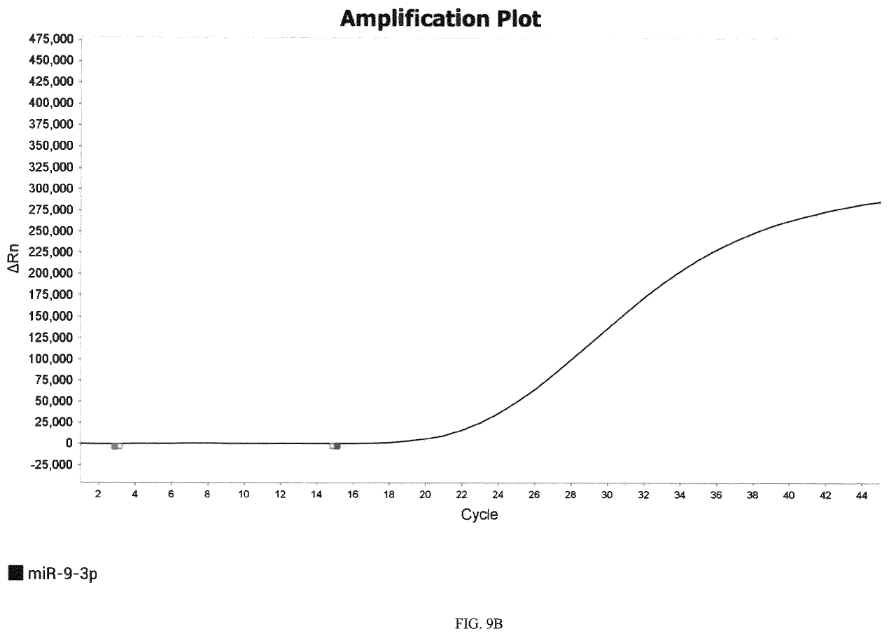

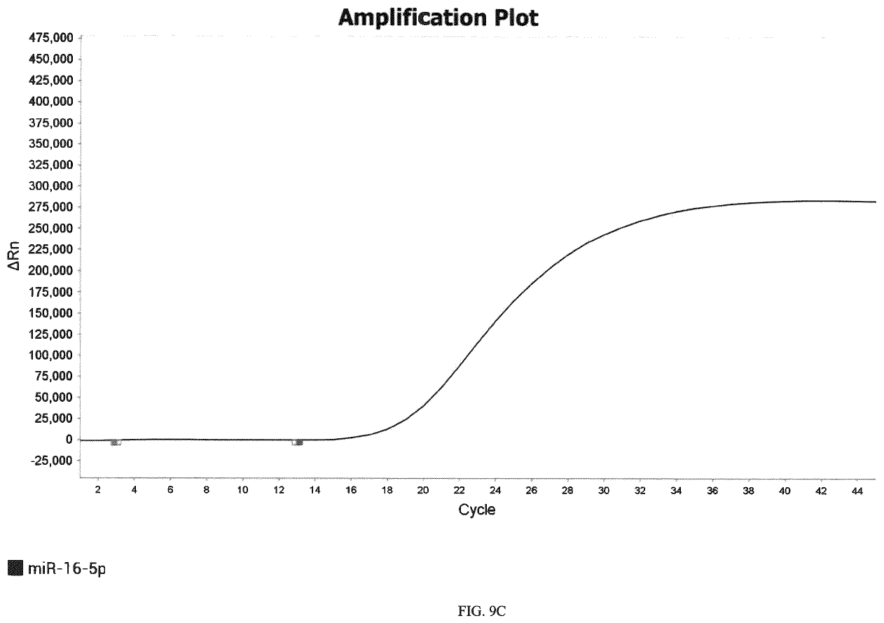

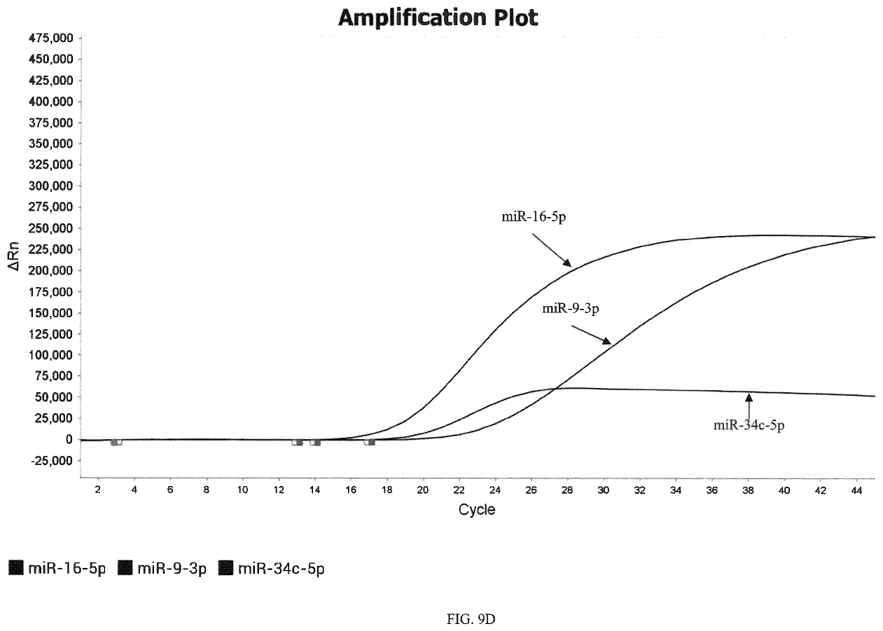

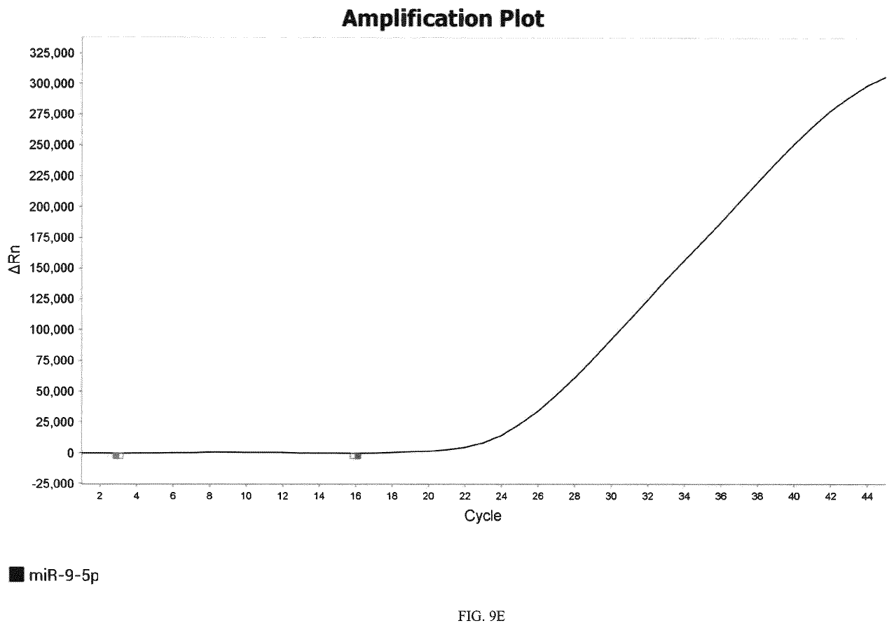

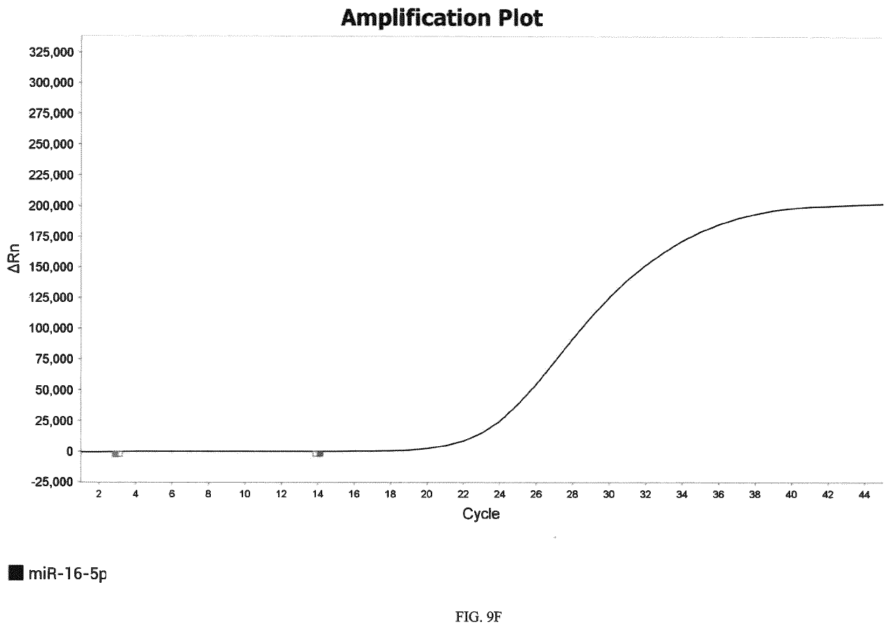

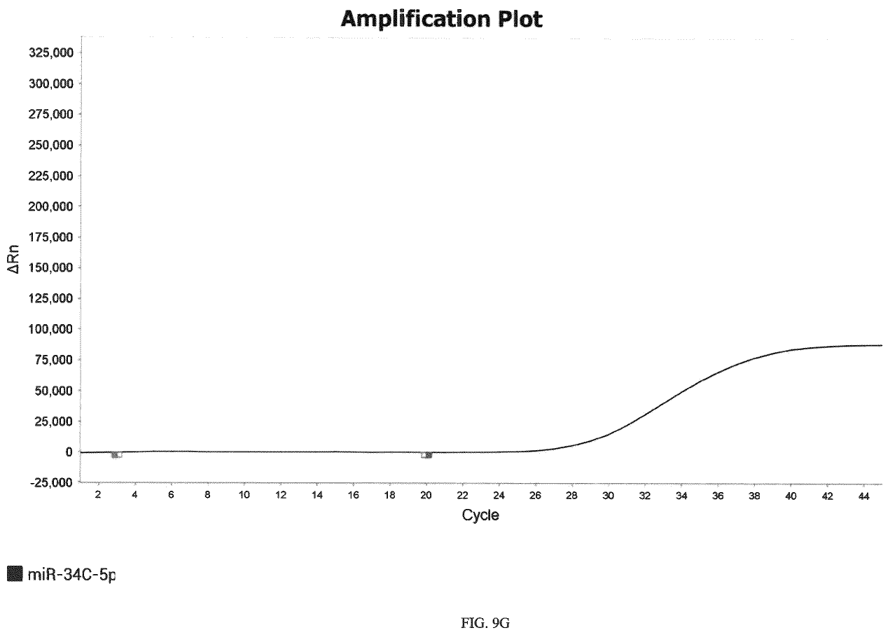

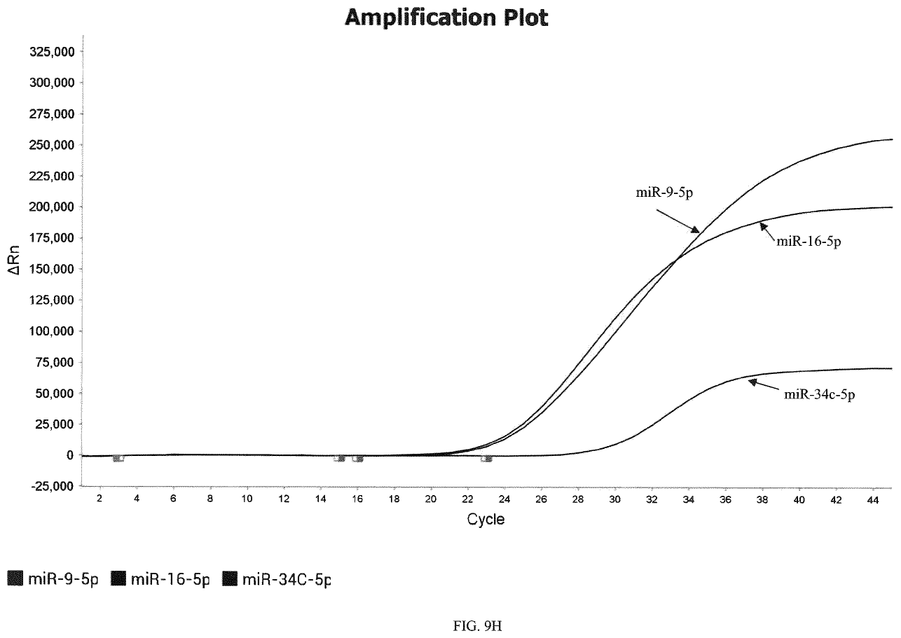

The serum samples were collected for the singleplex and triplex RT-qPCR quantification of the target miRNAs sample, and the verification of the triplex RT-qPCR was carried out using two target miRNAs combinations 3 and 4. The stem-loop reverse transcription primer having an 8 nt anchor sequence (SEQ ID No. 5) is selected for miR-16-5p, and its corresponding probe (SEQ ID No. 21) is labeled with VIC. The stem-loop reverse transcription primer having a 6 nt anchor sequence (SEQ ID No. 7) is selected for miR-9-3p, and its corresponding probe (SEQ ID No. 23) is labeled with ROX. The stem-loop reverse transcription primer having a 6 nt anchor sequence (SEQ ID No. 6) is selected for miR-34c-5p, and its corresponding probe (SEQ ID No. 22) is labeled with CY5. The stem-loop reverse transcription primer having a 11 nt anchor sequence (SEQ ID No. 8) is selected for miR-9-5p, and its corresponding probe (SEQ ID No. 24) is labeled with FAM. The results are shown in FIGS. 9A-H and Table 13. The results showed that there was no significant difference in amplification curve and Ct value between the singleplex and triplex quantifications of the target miRNAs.

TABLE 13
Singleplex and triplex quantifications of the target miRNAs

| Group | Group III | | | Group IV | | |
|---|---|---|---|---|---|---|
| Target | miR-34c-5p | miR-9-3p | miR-16-5p | miR-34c-5p | miR-16-5p | miR-9-5p |
| miRNA | | | | | | |
| Ct value of | 20.53 | 25.36 | 21.63 | 28.56 | 22.65 | 23.72 |
| the singleplex | | | | | | |
| Ct value of | 20.89 | 25.80 | 22.08 | 28.90 | 23.01 | 23.98 |
| the triplex | | | | | | |

Quadruplex System for Quantification of Four Target miRNAs

The quadruplex system includes the stem-loop reverse transcription primer combination having four stem-loop reverse transcription primers each of which demonstrates specificity to one of the target miRNAs, their corresponding forward/reverse primers, and their corresponding probes. The quadruplex RT-qPCR is performed to verify the sensitivity and specificity of the quadruplex stem-loop reverse transcription primer combination. In one embodiment, the target miRNA combination 5 includes miR-34c-5p, miR-16-5p, miR-9-3p, and miR-9-5p.

For the mixed RNA template 5 having all of miR-34c-5p, miR-16-5p, miR-9-3p, and miR-9-5p, the corresponding stem-loop reverse transcription primers having anchor sequences of different lengths were used to perform the quadruplex RT with the mixed RNA template 5, and ddH₂O as the negative control. Amplification results were observed to quantify the target miRNAs in the mixed RNA template 5. The specificity of each of the stem-loop reverse transcription primers having anchor sequences of different lengths was observed. The anchor sequence length for each of the four stem-loop reverse transcription primers in the quadruplex system is determined according to the specificity observed.

Based on the above steps, it was finally determined that, for miR-34c-5p, the stem-loop reverse transcription primer with the anchor sequence of 6 nt is selected, having the SEQ ID No. 6. For miR-16-5p, the stem-loop reverse transcription primer with the anchor sequence of 8 nt is selected, having the SEQ ID No. 5. For miR-9-3p, the stem-loop reverse transcription primer with the anchor sequence of 6 nt is selected, having the SEQ ID No. 7. For miR-9-5p, the stem-loop reverse transcription primer with the anchor sequence of 11 nt is selected, having the SEQ ID No. 8.

### Specificity of the Quadruplex Stem-Loop Reverse Transcription Primer Combinations

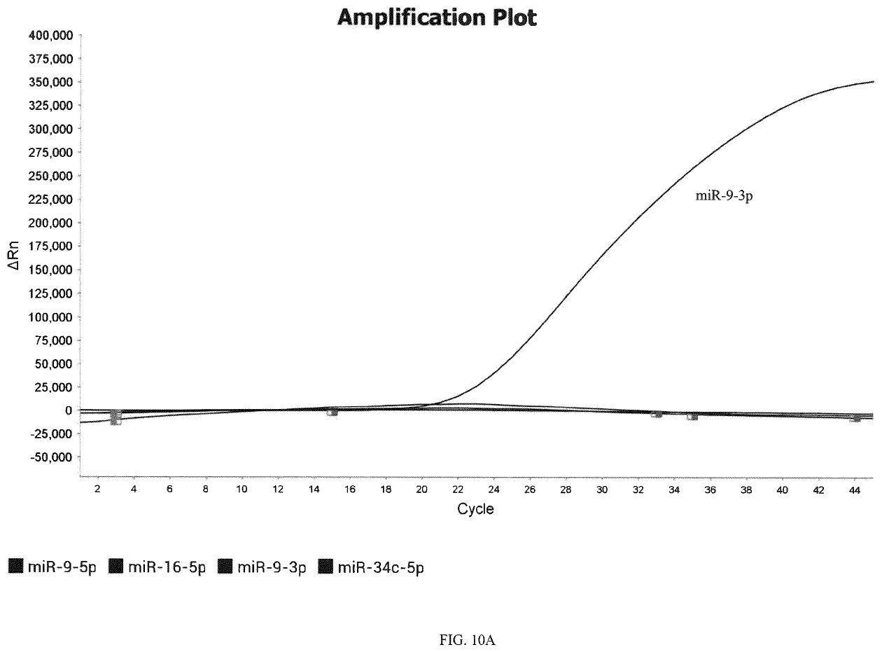

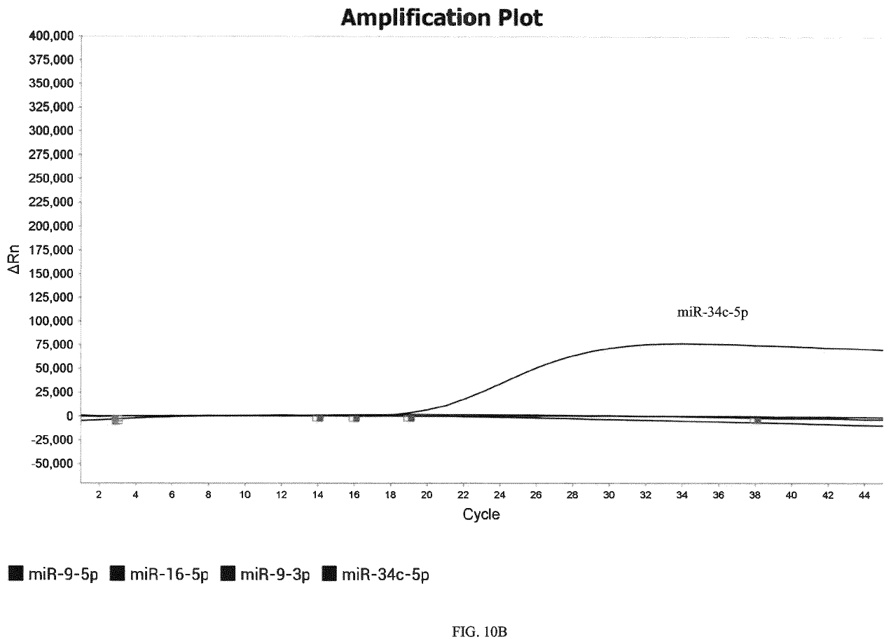

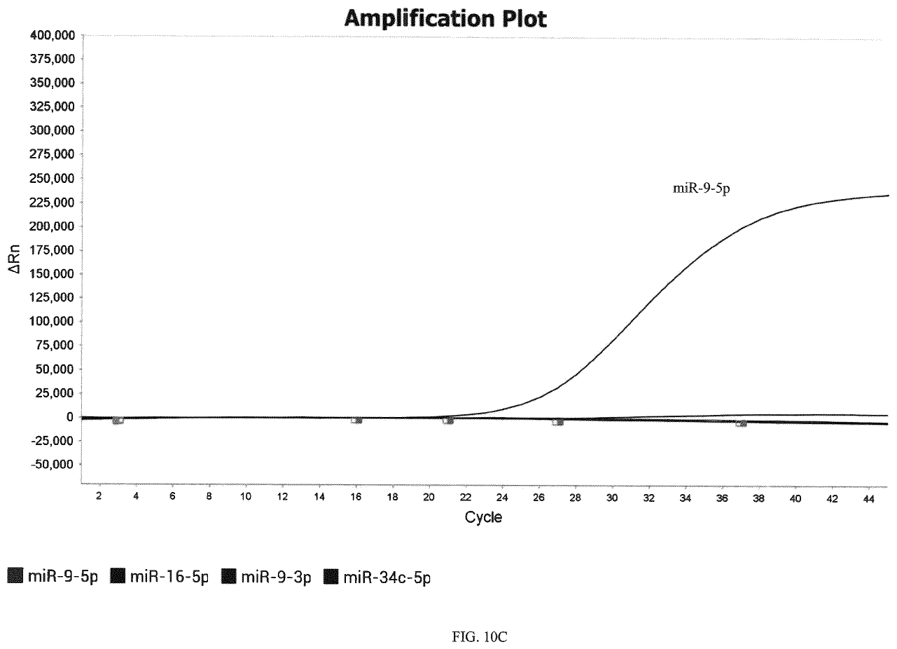

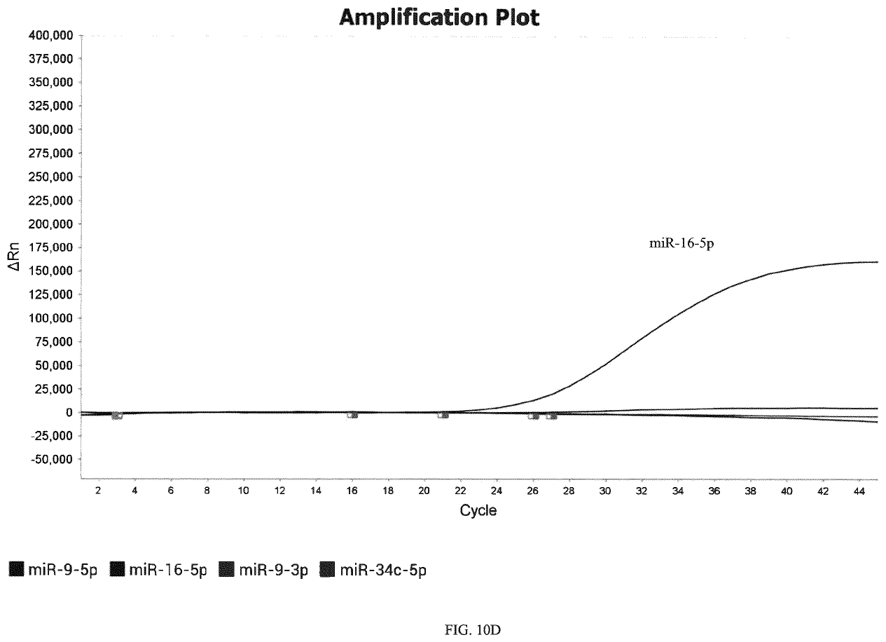

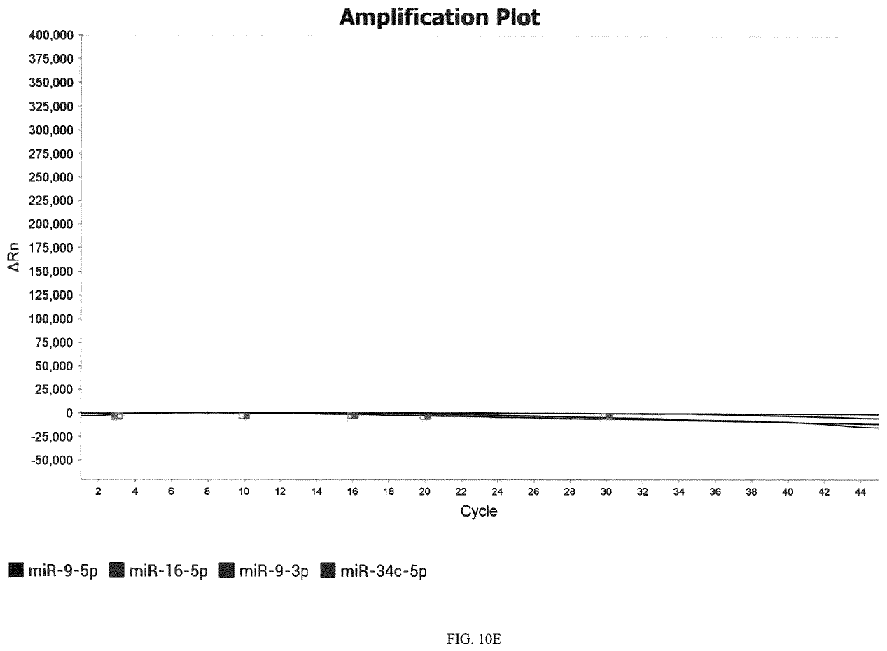

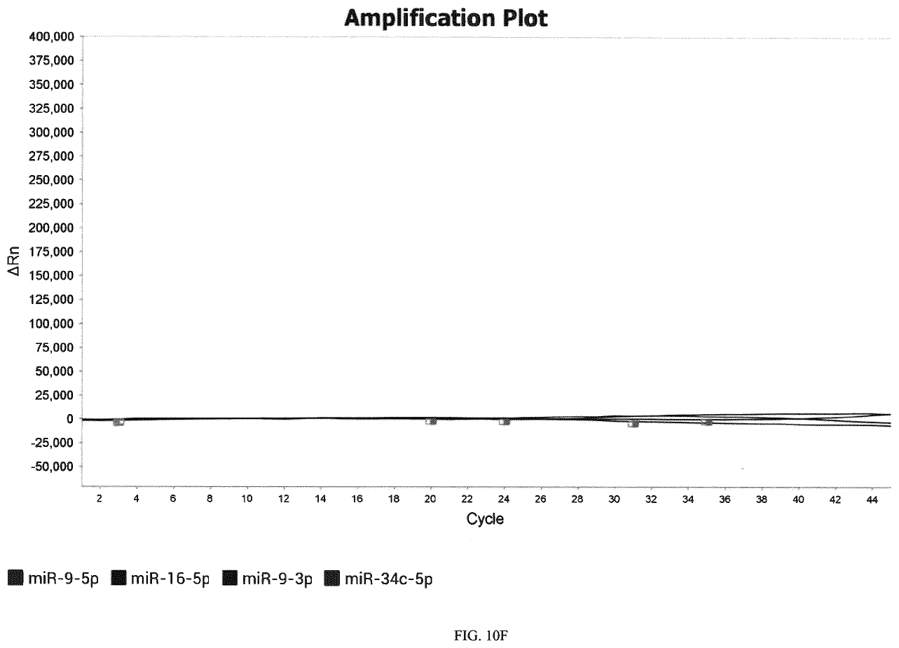

The mixed target miRNA template 5 undergoes the singleplex RT using each of the four corresponding stem-loop reverse transcription primers selected for the four target miRNAs, and then the quadruplex qPCR is formed on each of the singleplex reverse transcription products. FIGS. 10A-F shows the results of the four singleplex RT-quadruplex qPCRs, each using one corresponding stem-loop reverse transcription primer. In particular, it is shown in FIGS. 10A-D that the stem-loop reverse transcription primer for miR-9-3p (SEQ ID No. 7) specifically reverse transcribes miR-9-3p, the stem-loop reverse transcription primer for miR-34c-5p (SEQ ID No. 6) specifically reverse transcribes miR-34c-5p, and the stem-loop reverse transcription primer for miR-16-5p (SEQ ID No. 5) specifically reverse transcribes miR-16-5p, the stem-loop reverse transcription primer for miR-9-5p (SEQ ID No. 8) specifically reverse transcribes miR-9-5p. All four stem-loop reverse transcription primers show no signal in the negative control group using ddH₂O in FIGS. 10E-F.

Once the singleplex RT-quadruplex qPCRs show good specificity for each of the selected corresponding stem-loop reverse transcription primers, the quadruplex RT-qPCR is performed for determining if there is any undesired cross-reaction between the four corresponding stem-loop reverse transcriptions primers selected for the target miRNAs.

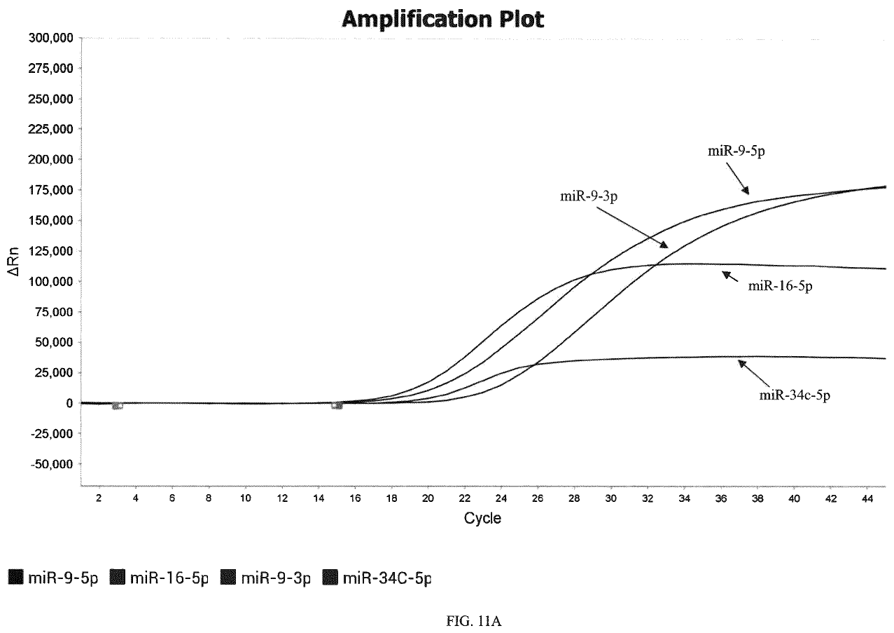

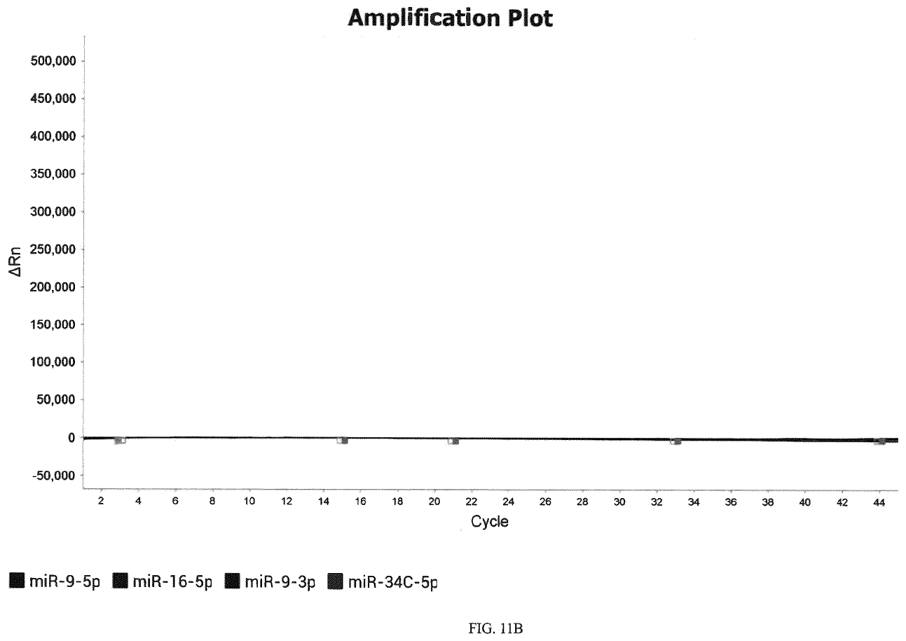

The mixed target miRNA templates 5 undergoes the quadruplex RT-qPCR using the quadruplex stem-loop reverse transcription primer combination containing all four corresponding stem-loop reverse transcription primers selected for the target miRNAs. FIGS. 11A-B shows the results of the quadruplex RT-qPCR of the mixed target miRNAs combination 5, with normal amplification curves for the target miRNAs in FIG. 11A and no signal in the negative control group using ddH₂O, as shown in FIG. 11B. This indicates that there exists no cross-reaction between the stem-loop reverse transcription primer for miR-9-3p (SEQ ID No. 7), the stem-loop reverse transcription primer for miR-34c-5p (SEQ ID No. 6), and the stem-loop reverse transcription primer for miR-16-5p (SEQ ID No. 5), and the stem-loop reverse transcription primer for miR-9-5p (SEQ ID No. 8).

### Sensitivity Test for the Duplex System

The quadruplex RT-qPCR was performed to determine the lowest detection limit, i.e. sensitivity, for each of the target miRNAs in the mixed target miRNA template combinations 5. Specifically, 1 pg of the mixed target miRNA template 5 was added, the quadruplex RT was performed, and the resulting product was diluted 10-fold in a gradient to a single copy number for the reverse transcription product.

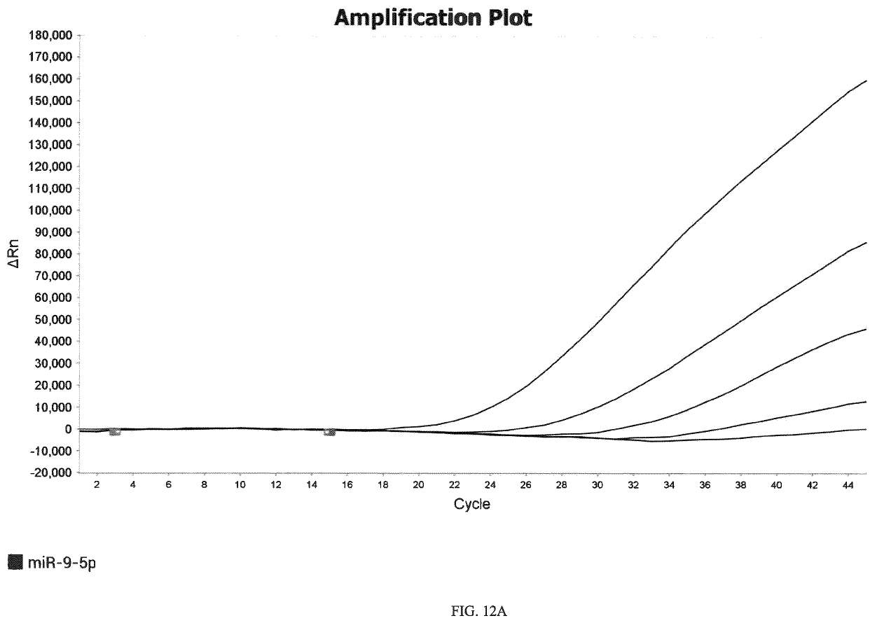

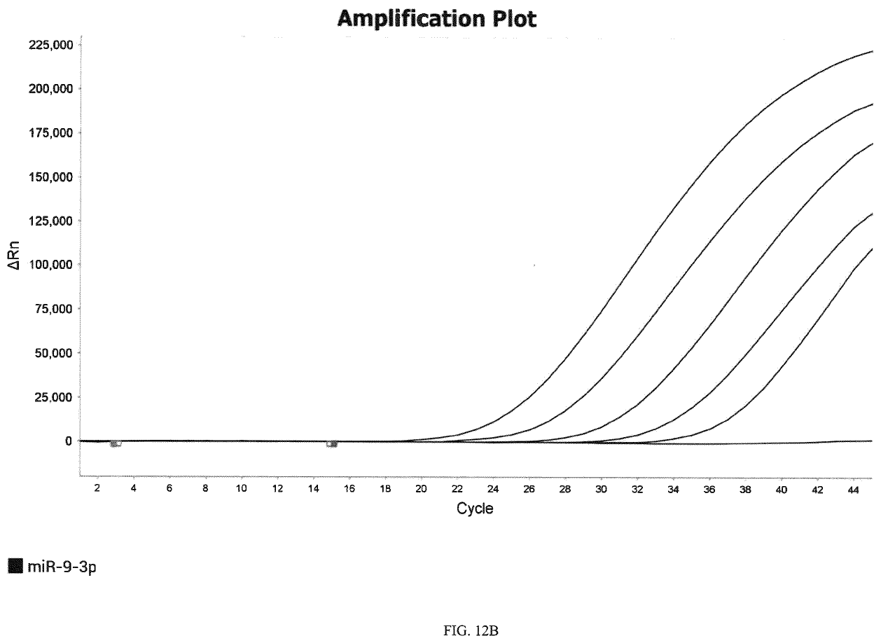

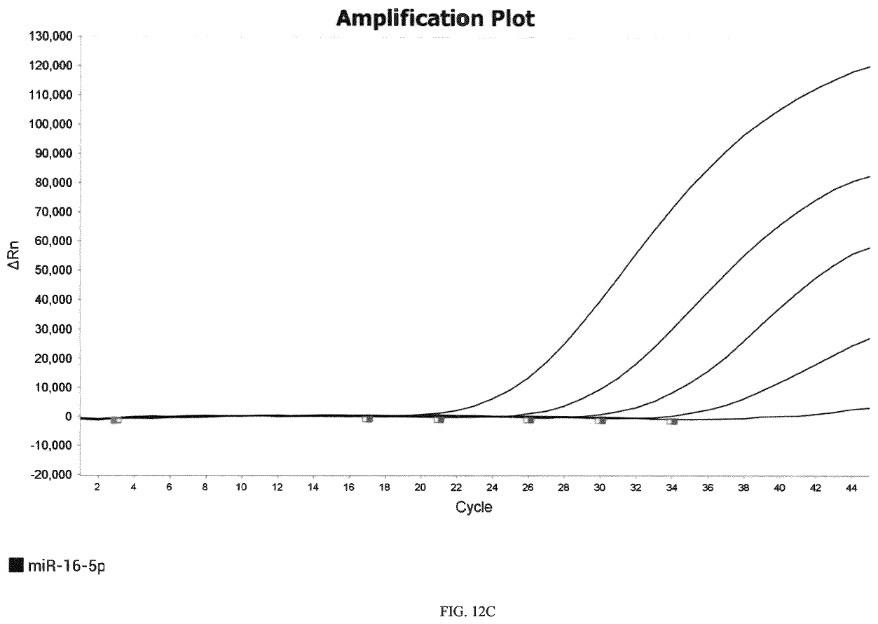

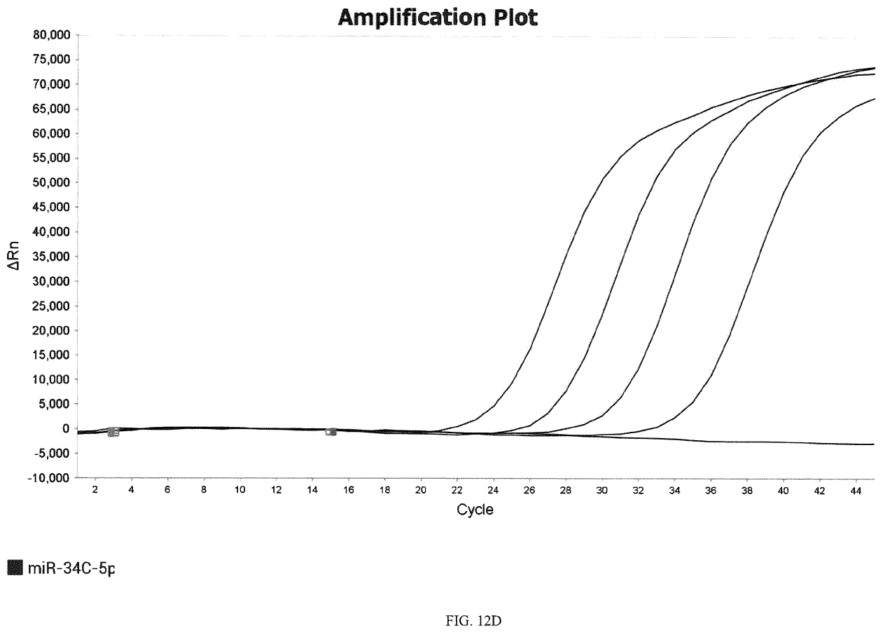

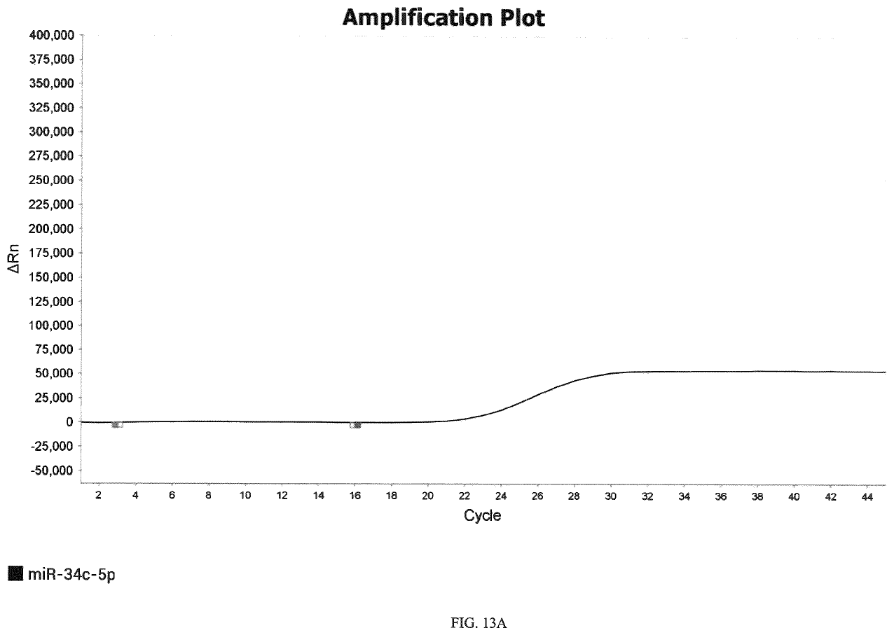

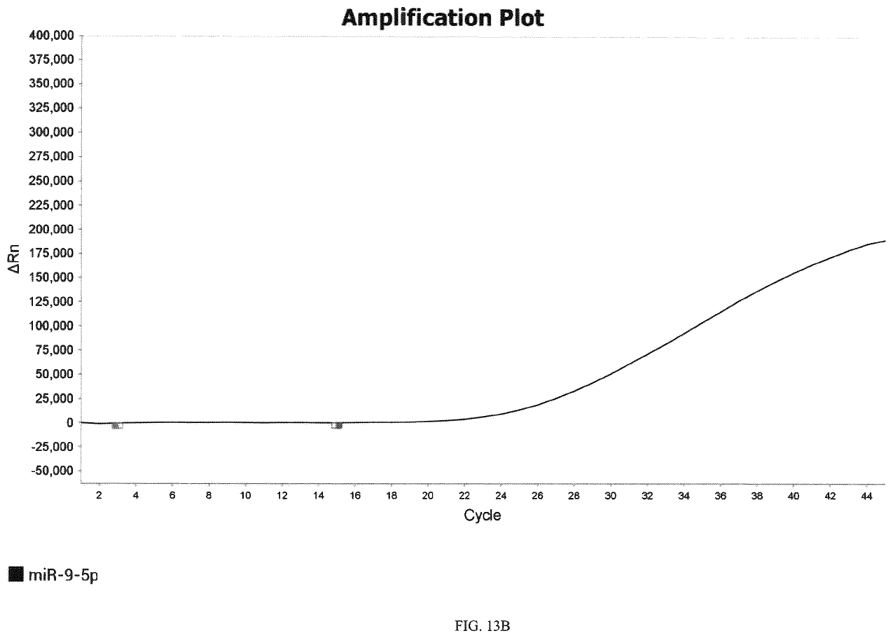

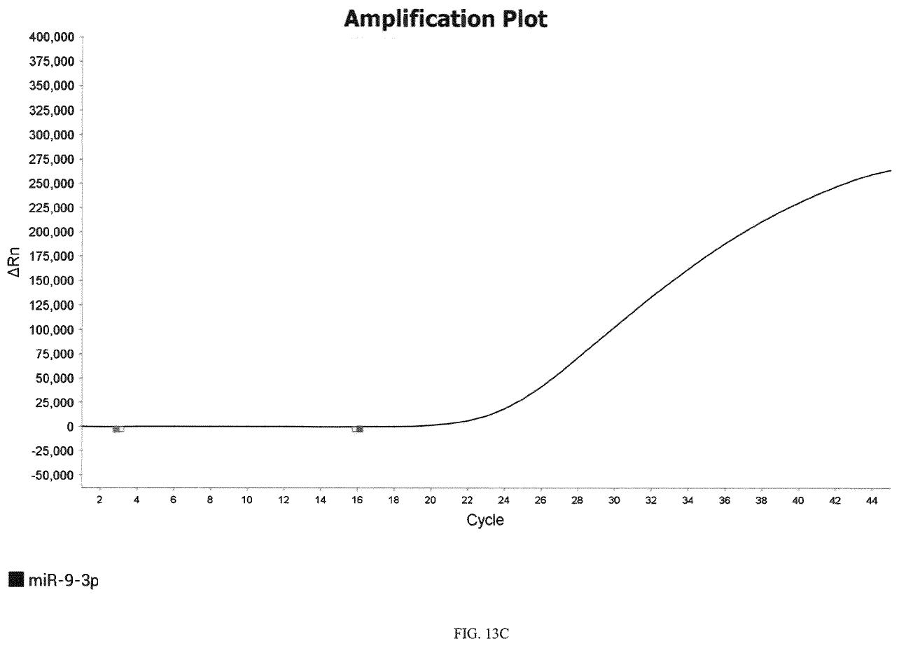

Then, all reverse transcription products obtained were subjected to the quadruplex qPCR. The results are shown in FIG. 12A-D (note: the amplification curves in each figure from left to right are the results at concentrations of 1 pg/μL, 0.1 pg/μL, 0.01 pg/μL, 1 fg/μL, and 0.1 fg/μL, respectively).

The results showed that, the detection limit of miR-16-5p was 0.1 fg/μL, the detection limit of miR-34c-5p was 0.1 fg/μL, and the detection limit of miR-9-3p was 0.1 fg/μL. In the combination 4, the detection limit of miR-9-5p was 1 fg/μL, the detection limit of miR-34c-5p was 1 fg, and the detection limit of miR-16-5p was 1 fg/μL.

Comparison of the Singleplex and Quadruplex Quantification of Target miRNAs

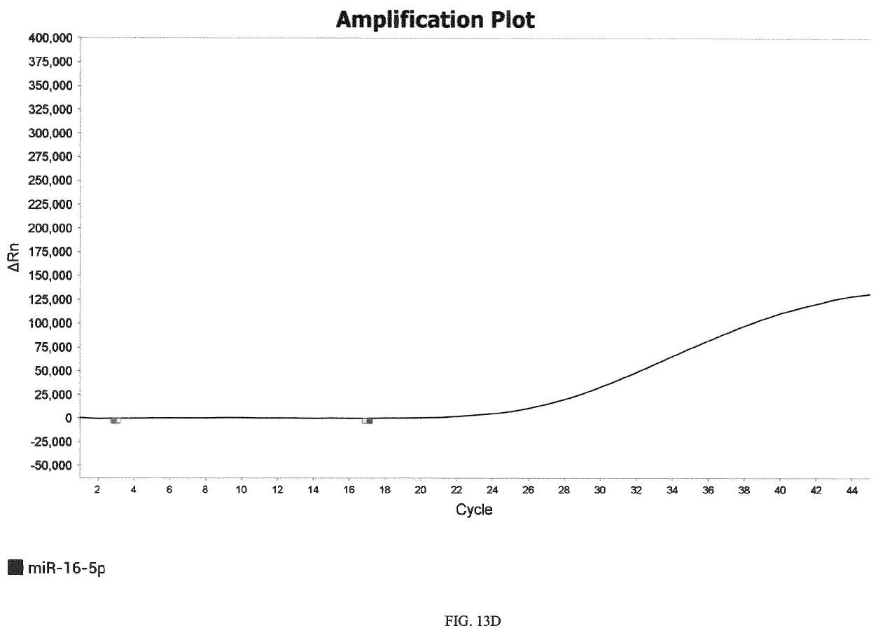

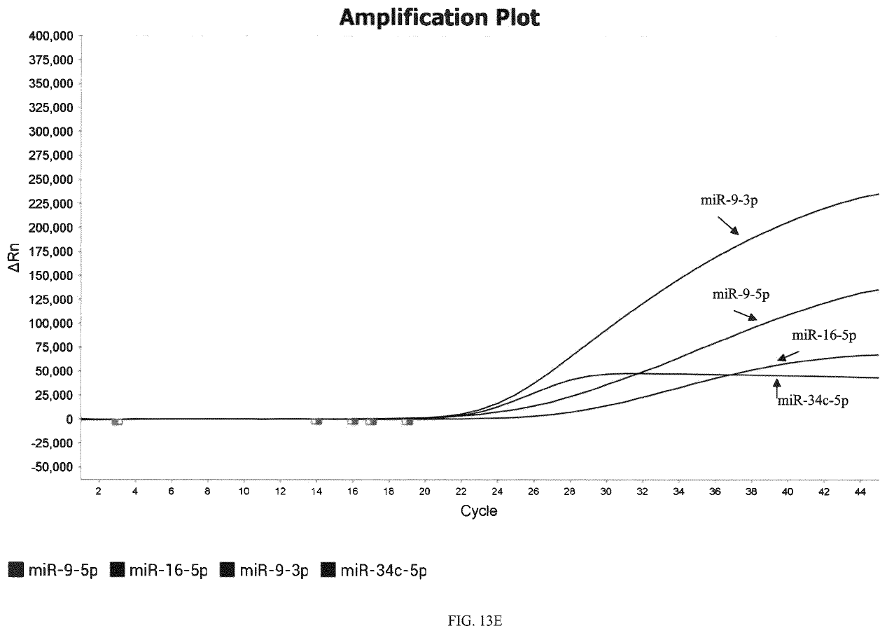

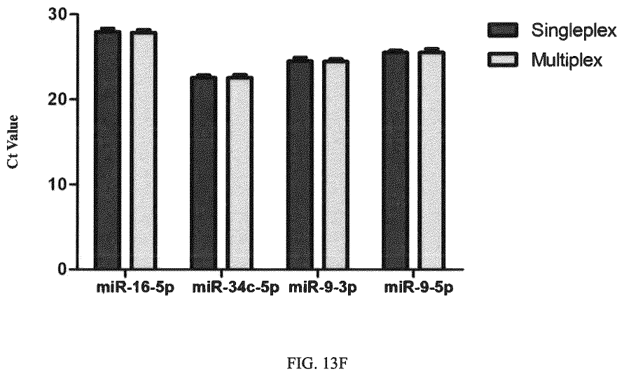

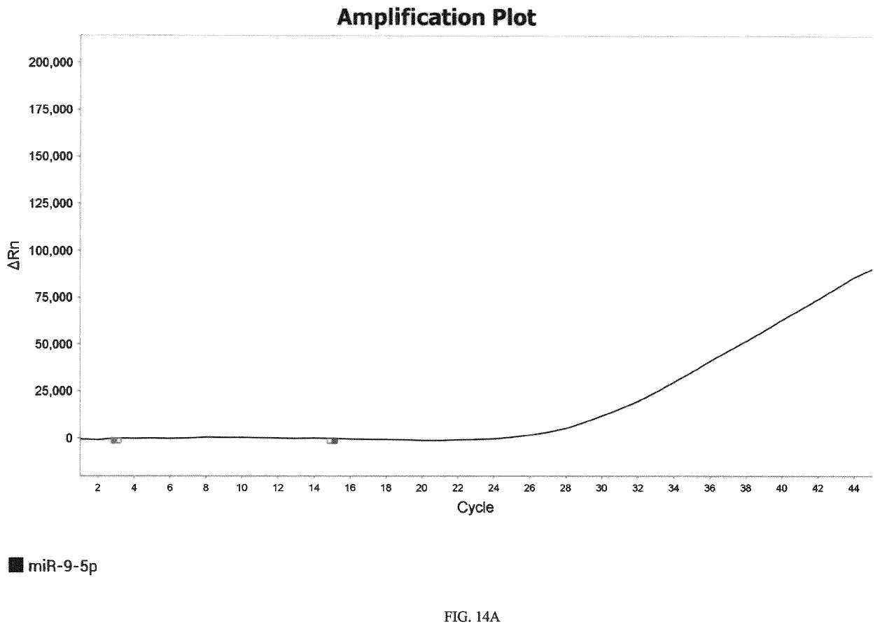

The serum samples were collected for the singleplex and quadruplex RT-qPCR quantification of the target miRNAs, and the verification of the quadruplex RT-qPCR was carried out using the mixed target miRNA template combination 5. The stem-loop reverse transcription primer having an 8 nt anchor sequence (SEQ ID No. 5) is selected for miR-16-5p, and its corresponding probe (SEQ ID No. 21) is labeled with VIC. The stem-loop reverse transcription primer having a 6 nt anchor sequence (SEQ ID No. 7) is selected for miR-9-3p, and its corresponding probe (SEQ ID No. 23) is labeled with ROX. The stem-loop reverse transcription primer having a 6 nt anchor sequence (SEQ ID No. 6) is selected for miR-34c-5p, and its corresponding probe (SEQ ID No. 22) is labeled with CY5. The stem-loop reverse transcription primer having a 11 nt anchor sequence (SEQ ID No. 8) is selected for miR-9-5p, and its corresponding probe (SEQ ID No. 24) is labeled with FAM. The results are shown in FIGS. 13A-F. The results showed that there was no significant difference in amplification curve and Ct value between the singleplex and quadruplex quantifications of the target miRNAs.

### Stability of Quadruplex RT-qPCR Quantification

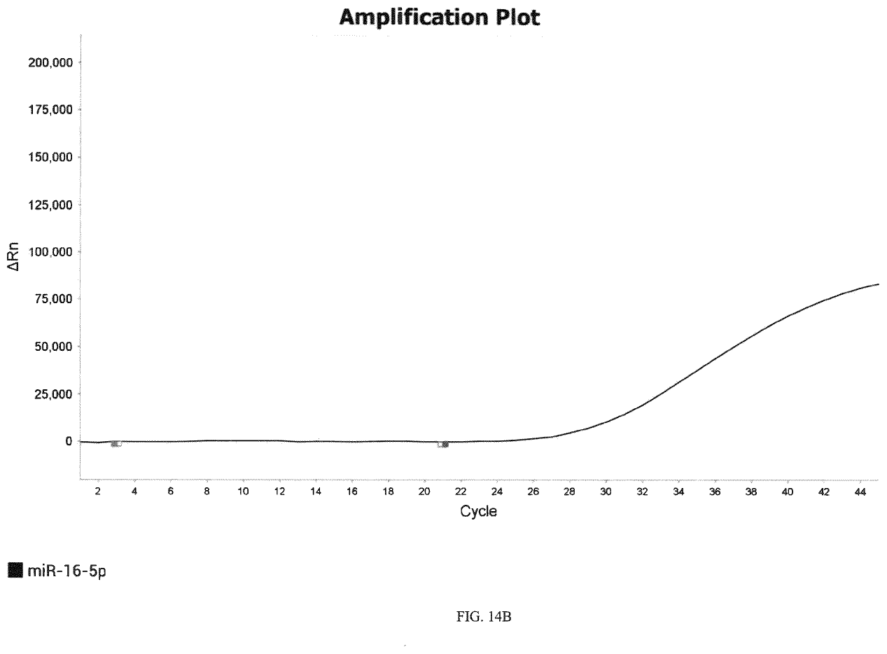

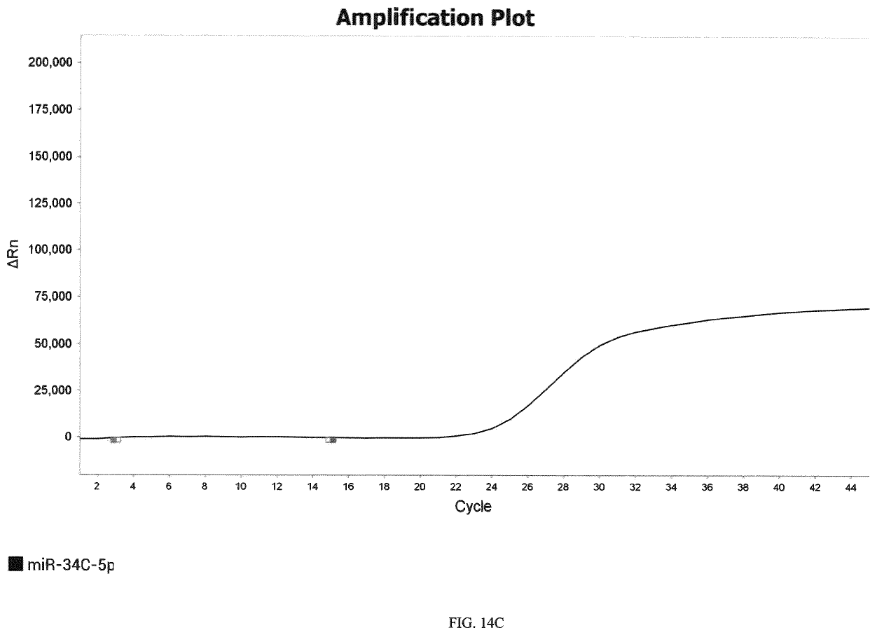

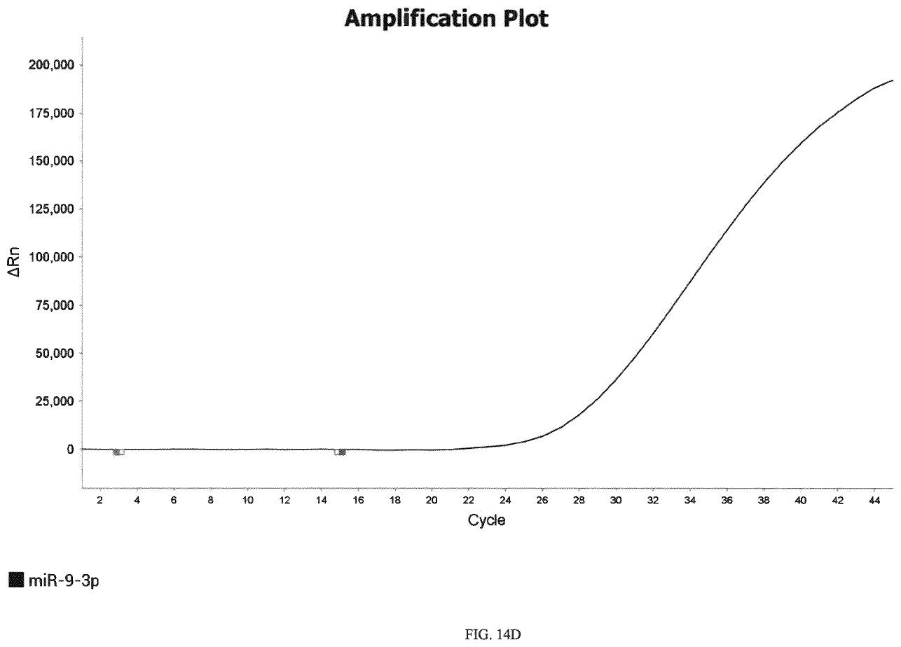

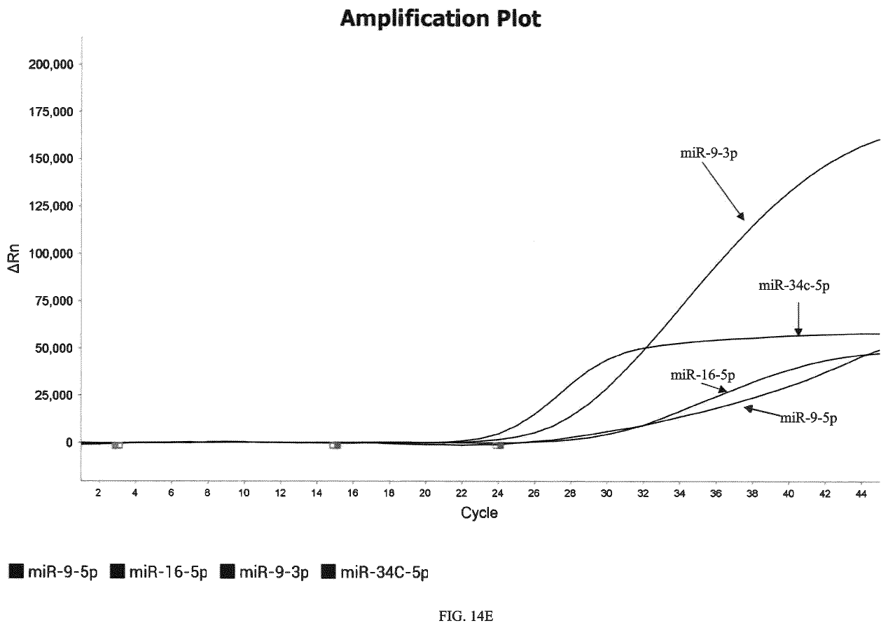

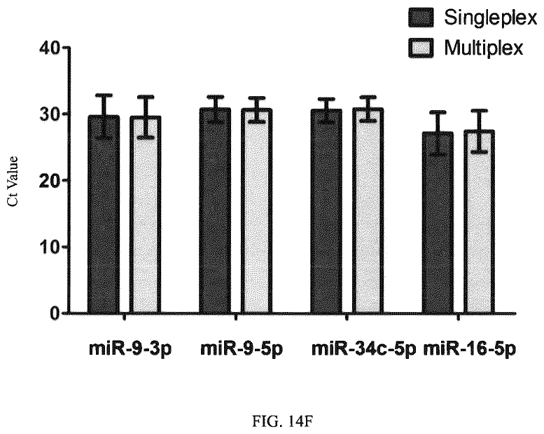

T The size of the test was expanded to 20 subjects for determining the stability of the quadruplex RT-qPCR quantification. The results are shown in FIG. 14A-F and Table 14. In particular, as reflected, the quadruplex stem-loop reverse transcription primer combination (SEQ ID No. 5-8) can accurately quantify the target miRNAs of miR-34c-5p, miR-9-3p, miR-16-5p and miR-9-5p using the quadruplex RT-qPCR.

TABLE 14
Singleplex and quadruplex quantifications of the target miRNAs

| Sample | miR-9-3p | miR-9-3p | miR-9-5p | miR-9-5p | miR-34C-5p | miR-34C-5p | miR-16-5p | miR-16-5p |
|---|---|---|---|---|---|---|---|---|
| No. | (Singleplex) | (Multiplex) | (Singleplex) | (Multiplex) | (Singleplex) | (Multiplex) | (Singleplex) | (Multiplex) |
| 1 | 23.626 | 23.794 | 23.618 | 23.727 | 23.618 | 23.727 | 23.618 | 23.727 |
| 2 | 23.618 | 23.727 | 31.567 | 31.334 | 31.567 | 31.334 | 29.695 | 31.334 |
| 3 | 23.085 | 23.702 | 30.9 | 31.545 | 30.79 | 31.645 | 29.67 | 29.335 |
| 4 | 31.306 | 31.832 | 31.285 | 31.165 | 30.58 | 31.64 | 29.655 | 29.585 |
| 5 | 31.42 | 31.31 | 32.005 | 31.58 | 31.08 | 31.895 | 29.945 | 30.01 |
| 6 | 23.618 | 23.727 | 32.145 | 30.495 | 31.18 | 31.935 | 29.835 | 30.135 |
| 7 | 31.567 | 31.334 | 31.35 | 31.235 | 31.09 | 31.925 | 29.99 | 29.605 |
| 8 | 31.2 | 30.9 | 32.39 | 32.145 | 31.3 | 31.525 | 29.83 | 29.93 |
| 9 | 29.45 | 29.335 | 30.15 | 30.135 | 31.46 | 31.055 | 31.05 | 31.08 |
| 10 | 32.04 | 31.645 | 32.06 | 31.935 | 30.94 | 31.175 | 29.79 | 29.93 |
| 11 | 31.43 | 31.285 | 31.26 | 31.35 | 31.33 | 31.525 | 31.3 | 31.175 |
| 12 | 29.73 | 29.585 | 29.67 | 29.605 | 31.11 | 31.165 | 26.167 | 26.04 |
| 13 | 32.04 | 31.64 | 31.96 | 31.925 | 29.73 | 29.67 | 25.042 | 25.364 |
| 14 | 32.14 | 32.005 | 31.71 | 31.545 | 31.12 | 31.18 | 25.374 | 25.991 |
| 15 | 29.9 | 30.01 | 29.72 | 29.695 | 31.76 | 31.58 | 24.178 | 26.04 |
| 16 | 32.39 | 31.895 | 31.1 | 31.235 | 29.6 | 29.655 | 23.384 | 25.364 |
| 17 | 31.04 | 30.94 | 29.67 | 29.835 | 31.15 | 31.09 | 22.14 | 25.991 |
| 18 | 30.64 | 30.58 | 31.09 | 31.46 | 30.52 | 30.495 | 24.444 | 22.809 |
| 19 | 29.83 | 29.83 | 30.79 | 30.79 | 29.81 | 29.945 | 23.195 | 21.262 |
| 20 | 31.47 | 31.055 | 29.7 | 29.99 | 31.42 | 31.3 | 24.153 | 23.989 |
| Mean | 29.577 | 29.5065 | 30.707 | 30.6363 | 30.5577 | 30.7730 | 27.1227 | 27.4348 |

Specificity of the Multiplex Stem-Loop Reverse Transcription Primer Combination with Varied Anchor Sequence Lengths

The accuracy of the multiplex RT-qPCR quantification would be compromised if the stem-loop reverse transcription primers are modified. In one embodiment, a mixed RNA template including miR-16-5p, miR-34c-5p, miR-9-3p, miR-9-5p was used to perform singleplex reverse transcription. An alternate stem-loop reverse transcription primer combination is selected. Among them, miR-16-5p chose a reverse transcription primer with an anchor sequence length of 11 nt with the stem-loop 1 (SEQ ID No. 62), miR-34c-5p chose a reverse transcription primer with an anchor sequence length of 8 nt with the stem-loop 2 (SEQ ID No. 65), miR-9-3p chose a reverse transcription primer with an anchor sequence length of 8 nt with the stem-loop 3 (SEQ ID No. 67), and miR-9-5p chose a reverse transcription primer with an anchor sequence length of 6 nt with the stem-loop 4 (SEQ ID No. 69).

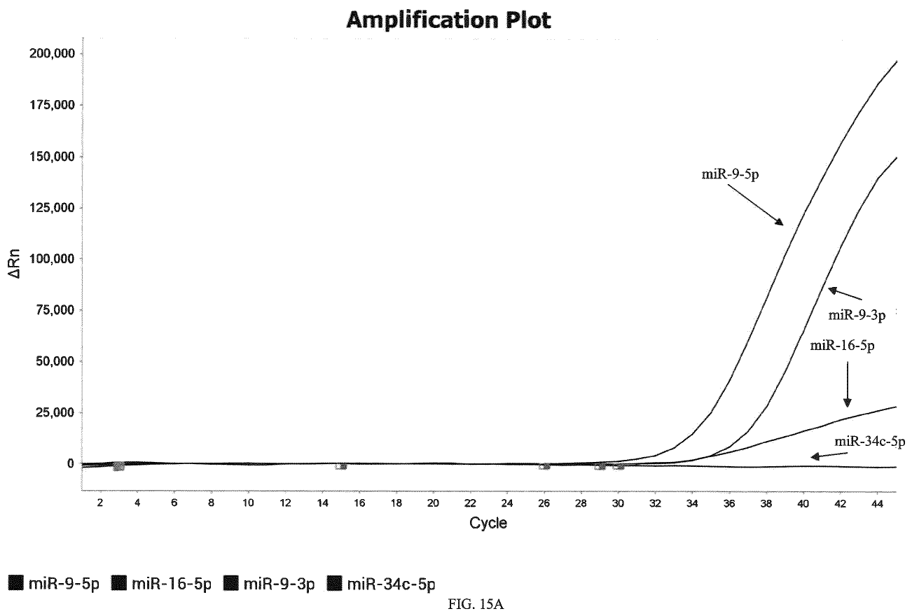

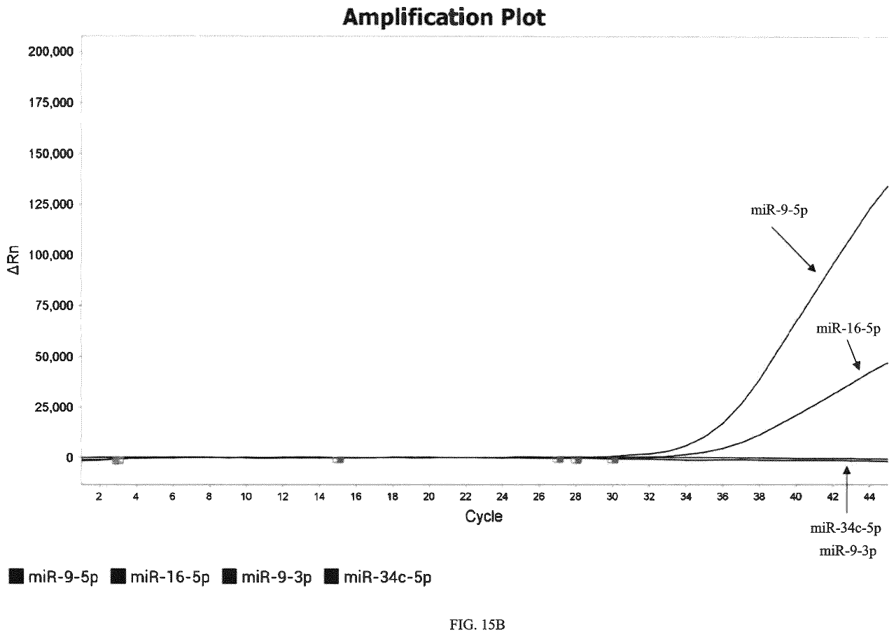

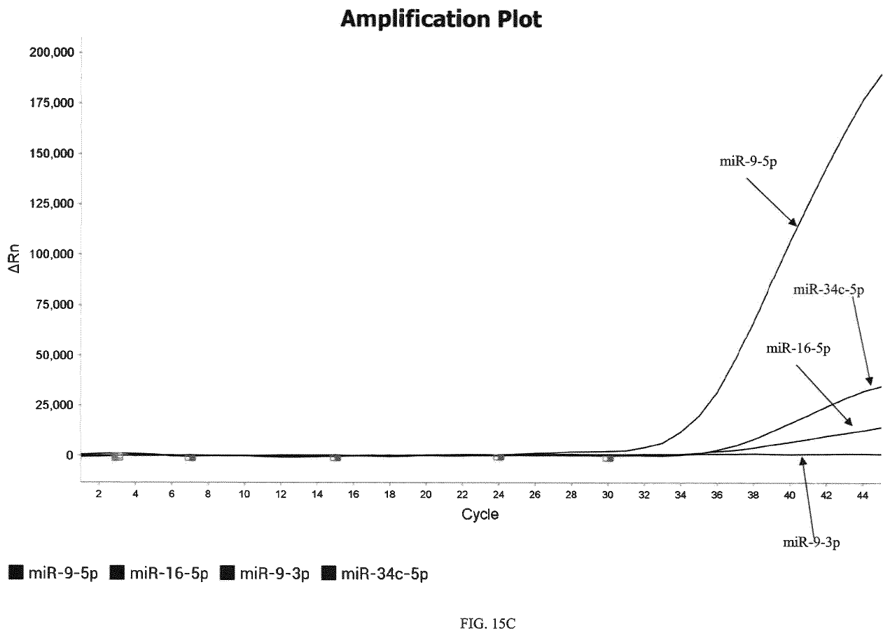

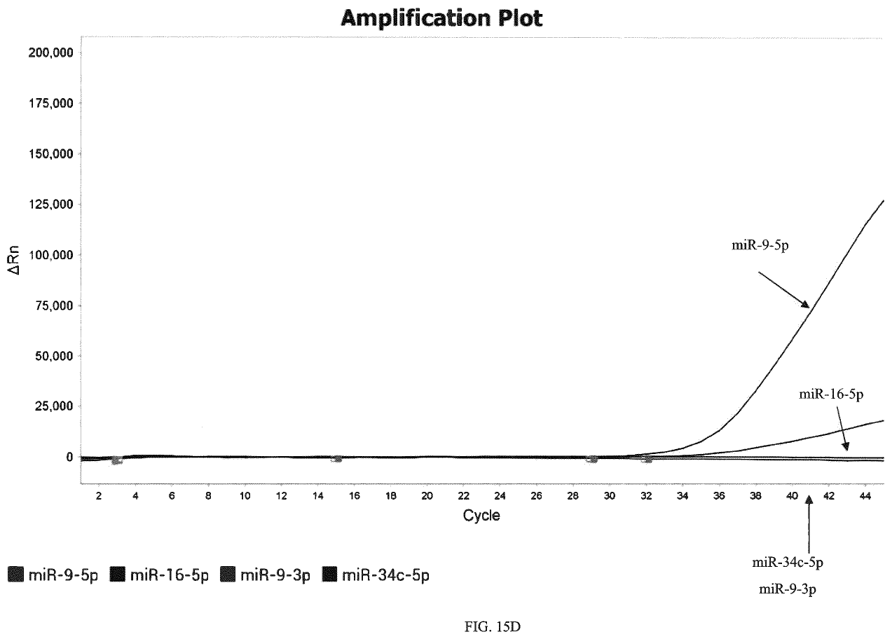

Then, the multiplex qPCR was performed to determine whether there was any cross-reaction among the quantification of the four target miRNAs, as so to verify the specificity of the alternate stem-loop reverse transcription primer combination, and to compare the Ct values of each of the target miRNAs. The results are shown in FIG. 15A-D and Table 15.

TABLE 15
The singleplex and quadruplex quantifications by the
alternate stem-loop reverse transcription primer

| Target miRNAs | miR-9-3p | miR-16-5p | miR-34c-5p | miR-9-5p |
|---|---|---|---|---|
| Ct value of the | 30.25 | 29.82 | 33.61 | 33.60 |
| singleplex | | | | |
| Ct value of the | 33.30 | 33.60 | 31.23 | 28.39 |
| quadruplex | | | | |

By observing the amplification curves of the target miRNAs, it was found that when miR-9-3p was reverse transcribed alone, there was signal interference from miR-16-5p and miR-9-5p; when miR-9-5p was reverse transcribed alone, there was signal interference from miR-16-5p; when miR-16-5p was reverse transcribed alone, there was signal interference from miR-9-5p; when miR-34c-5p was reverse transcribed alone, there was signal interference from miR-16-5p and miR-9-5p; and there was a significant difference in Ct values between single and quadruple quantitative detection.

The multiplex target miRNA quantification method of the present invention is also applicable to other miRNA targets, as long as the homology of the miRNA sequence is low. The 3′ end of the miRNA sequence cannot contain two or more consecutive identical bases to avoid non-specific binding of the stem-loop reverse transcription primers and affecting the experimental results. In the construction of the multiplex target miRNA quantification system, the stem-loop reverse transcription primers with different stem-loop sequences are used to obtain cDNA products with significantly different sequences. Specific primers and probes are designed for the cDNA to verify the sensitivity and specificity of the multiplex system. Once the multiplex RT-qPCR with the multiplex stem-loop reverse transcription primer combination is verified for its multi-specificity and sensitivity, and then the sample quantification can be performed in desired settings, e.g. clinic diagnosis of diseases.

In one embodiment, miRNAs other than miR-16-5p, miR-34c-5p, miR-9-3p, and miR-9-5p can be used as target miRNAs for using in determination of Alzheimer's Disease. Each target miRNA has a corresponding stem-loop reverse transcription primer for its reverse transcription, and a corresponding forward primer, a corresponding reverse primer, and a corresponding probe having a fluorescent reporter group and a quencher group for qPCR of the reverse transcription products. Table 16 shows the target miRNAs and their corresponding forward/reverse primers and probes for qPCR.

TABLE. 16
Target miRNAs for Alzheimer′s Disease and their corresponding stem-loop
reverse transcription primers, forward/reverse primers and probes for qPCR

| | | Stem-loop reverse | Anchor | Forward | | Reverse |
|---|---|---|---|---|---|---|
| Target | miRNA | transcription | sequence | primer | Probe | primer |
| miRNA | sequence | primer (5′-3′) | (nt) | (5′-3′) | (5′-3′) | (5′-3′) |
| hsa- | UGUAGUGU | GTCGTATCCAGTGCA | 8 | gccgccTGTAG | FAM- | GTGCAGGGT |
| miR- | UUCCUACU | GGGTCCGAGGTATTC | | TGTTTCC | CTTTATGGAG | CCGAGGT |
| 142-3p | UUAUGGA | GCACTGGATACGACT | | (SEQ ID No. | TCGTATCCAG- | (SEQ ID No. |
| | (SEQ ID No. | CCATAAA (SEQ ID | | 138) | MGB (SEQ ID | 50) |
| | 70) | No. 104) | | | No. 172) | |
| hsa- | UAACAGUC | GTCGTATCCAGTGCA | 8 | cgccgccTAAC | ROX- | GTGCAGGGT |
| miR- | UACAGCCA | GGGTCCGAGGTATTC | | AGTCTAC | GCCATGGTC | CCGAGGT |
| 132-3p | UGGUCG | GCACTGGATACGACC | | (SEQ ID No. | GGTCGT-MGB | (SEQ ID No. |
| | (SEQ ID No. | GACCATG (SEQ ID | | 139) | (SEQ ID No. | 50) |
| | 71) | No. 105) | | | 173) | |
| hsa- | UGAGAACU | GTCGTATCCAGTGCA | 8 | cgcgcgTGAGA | VIC- | GTGCAGGGT |
| miR- | GAAUUCCA | GGGTCCGAGGTATTC | | ACTGAAT | TCCATGGGTT | CCGAGGT |
| 146a- | UGGGUU | GCACTGGATACGAC | | (SEQ ID No. | GTCGTAT- | (SEQ ID No. |
| 5p | (SEQ ID No. | AACCCATG (SEQ ID | | 140) | MGB (SEQ ID | 50) |
| | 72) | No. 106) | | | No. 174) | |
| hsa- | AAAAGCUG | GTCGTATCCAGTGCA | 8 | ccgcgAAAAG | CY5- | GTGCAGGGT |
| miR- | GGUUGAGA | GGGTCCGAGGTATTC | | CTGGGT | TGAGAGGGC | CCGAGGT |
| 320a- | GGGCGA | GCACTGGATACGACT | | (SEQ ID No. | GAGTCGT- | (SEQ ID No. |
| 3p | (SEQ ID No. | CGCCCTC (SEQ ID | | 141) | MGB (SEQ ID | 50) |
| | 73) | No. 107) | | | No. 175) | |
| hsa- | GCCUGCUG | GTCGTATCCAGTGCA | 8 | ttcGCCTGCTG | VIC- | GTGCAGGGT |
| miR- | GGGUGGAA | GGGTCCGAGGTATTC | | GGGT (SEQ | GGAACCTGG | CCGAGGT |
| 370-3p | CCUGGU | GCACTGGATACGAC | | ID No. 142) | TGTCGT-MGB | (SEQ ID No. |
| | (SEQ ID No. | ACCAGGTT (SEQ ID | | | (SEQ ID No. | 50) |
| | 74) | No. 108) | | | 176) | |
| hsa- | GCAGUCCA | GTCGTATCCAGTGCA | 8 | tccgccGCAGT | ROX- | GTGCAGGGT |
| miR- | UGGGCAUA | GGGTCCGAGGTATTC | | CCAT (SEQ | GGGCATATA | CCGAGGT |
| 455-3p | UACAC | GCACTGGATACGAC | | ID No. 143) | CACGTCG- | (SEQ ID No. |
| | (SEQ ID No. | GTGTATAT (SEQ ID | | | MGB (SEQ ID | 50) |
| | 75) | No. 109) | | | No. 177) | |
| hsa- | UCCUGUAC | GTCGTATCCAGTGCA | 8 | gccgccTCCTG | FAM- | GTGCAGGGT |
| miR- | UGAGCUGC | GGGTCCGAGGTATTC | | TACTGA | TGCCCCGAG | CCGAGGT |
| 486-5p | CCCGAG | GCACTGGATACGACC | | (SEQ ID No. | GTCGTATC- | (SEQ ID No. |
| | (76) | TCGGGGC (SEQ ID | | 144) | MGB (SEQ ID | 50) |
| | | No. 110) | | | No. 178) | |
| hsa- | AAAAGUGC | GTCGTATCCAGTGCA | 8 | gccgccAAAAG | CY5- | GTGCAGGGT |
| miR- | UUACAGUG | GGGTCCGAGGTATTC | | TGCTTACA | GTGCAGGTA | CCGAGGT |
| 106a- | CAGGUAG | GCACTGGATACGACC | | (SEQ ID No. | GGTCGTAT- | (SEQ ID No. |
| 5p | (SEQ ID No. | TACCTGC (SEQ ID | | 145) | MGB (SEQ ID | 50) |
| | 77) | No. 111) | | | No. 179) | |
| hsa- | AGCAGCAU | GTCGTATCCAGTGCA | 8 | cgcgcAGCAG | FAM- | GTGCAGGGT |
| miR- | UGUACAGG | GGGTCCGAGGTATTC | | CATTGT (SEQ | ACAGGGCTA | CCGAGGT |
| 103a- | GCUAUGA | GCACTGGATACGACT | | ID No. 146) | TGAGTCGT- | (SEQ ID No. |
| 3p | (SEQ ID No. | CATAGCC (SEQ ID | | | MGB (SEQ ID | 50) |
| | 78) | No. 112) | | | No. 180) | |
| hsa- | UCGUACCG | GTCGTATCCAGTGCA | 6 | ttcgcgcTCGTA | ROX- | GTGCAGGGT |
| miR- | UGAGUAAU | GGGTCCGAGGTATTC | | CCGT (SEQ ID | AGTAATAAT | CCGAGGT |
| 126-3p | AAUGCG | GCACTGGATACGACC | | No. 147) | GCGGTCGTAT | (SEQ ID No. |
| | (SEQ ID No. | GCATT (SEQ ID No. | | | CC-MGB (SEQ | 50) |
| | 79) | 113) | | | ID No. 181) | |
| hsa- | UAGCAGCA | GTCGTATCCAGTGCA | 8 | cgcgcTAGCAG | VIC- | GTGCAGGGT |
| miR- | CAUCAUGG | GGGTCCGAGGTATTC | | CACAT (SEQ | TGGTTTACAG | CCGAGGT |
| 15b-5p | UUUACA | GCACTGGATACGACT | | ID No. 148) | TCGTATCCAG | (SEQ ID No. |
| | (SEQ ID No. | GTAAACC (SEQ ID | | | T-MGB (SEQ | 50) |
| | 80) | No. 114) | | | ID No. 182) | |
| hsa- | UCUCACAC | GTCGTATCCAGTGCA | 8 | gcgcgcTCTCA | CY5- | GTGCAGGGT |
| miR- | AGAAAUCG | GGGTCCGAGGTATTC | | CACAGA | TCGCACCCGT | CCGAGGT |
| 342-3p | CACCCGU | GCACTGGATACGAC | | (SEQ ID No. | GTCGTAT- | (SEQ ID No. |
| | (SEQ ID No. | ACGGGTGC (SEQ ID | | 149) | MGB (SEQ ID | 50) |
| | 81) | No. 115) | | | No. 183) | |
| hsa- | AGCAGCAU | GTCGTATCCAGTGCA | 8 | cgcgcgAGCAG | FAM- | GTGCAGGGT |
| miR- | UGUACAGG | GGGTCCGAGGTATTC | | CATTGTA | CAGGGCTAT | CCGAGGT |
| 107-3p | GCUAUCA | GCACTGGATACGACT | | (SEQ ID No. | CAGTCGTAT- | (SEQ ID No. |
| | (SEQ ID No. | GATAGCC (SEQ ID | | 150) | MGB (SEQ ID | 50) |
| | 82) | No. 116) | | | No. 184) | |
| hsa- | AACAUUCA | GTCGTATCCAGTGCA | 8 | cgcgcgAACAT | VIC- | GTGCAGGGT |
| miR- | ACCUGUCG | GGGTCCGAGGTATTC | | TCAACCT | GTCGGTGAG | CCGAGGT |
| 181c- | GUGAGU | GCACTGGATACGAC | | (SEQ ID No. | TGTCGTAT- | (SEQ ID No. |
| 5p | (SEQ ID No. | ACTCACCG (SEQ ID | | 151) | MGB (SEQ ID | 50) |
| | 83) | No. 117) | | | No. 185) | |
| hsa- | UAGCUUAU | GTCGTATCCAGTGCA | 8 | gcgcgcgTAGC | ROX- | GTGCAGGGT |
| miR- | CAGACUGA | GGGTCCGAGGTATTC | | TTATCAGA | CTGATGTTGA | CCGAGGT |
| 21-5p | UGUUGA | GCACTGGATACGACT | | (SEQ ID No. | GTCGTATCCA | (SEQ ID No. |
| | (SEQ ID No. | CAACATC (SEQ ID | | 152) | G-MGB (SEQ | 50) |
| | 84) | No. 118) | | | ID No. 186) | |
| hsa- | CGCAUCCC | GTCGTATCCAGTGCA | 8 | gcggCGCATC | FAM- | GTGCAGGGT |
| miR- | CUAGGGCA | GGGTCCGAGGTATTC | | CCCTA (SEQ | GGGCATTGG | CCGAGGT |
| 324-5p | UUGGUGU | GCACTGGATACGAC | | ID No. 153) | TGTGTCGT- | (SEQ ID No. |
| | (SEQ ID No. | ACACCAAT (SEQ ID | | | MGB (SEQ ID | 50) |
| | 85) | No. 119) | | | No. 187) | |
| hsa- | CUAGACUG | GTCGTATCCAGTGCA | 8 | cgccgccCTAG | VIC- | GTGCAGGGT |
| miR- | AAGCUCCU | GGGTCCGAGGTATTC | | ACTGAA | GCTCCTTGAG | CCGAGGT |
| 151a- | UGAGG | GCACTGGATACGACC | | (SEQ ID No. | GGTCGT-MGB | (SEQ ID No. |
| 3p | (SEQ ID No. | CTCAAGG (SEQ ID | | 154) | (SEQ ID No. | 50) |
| | 86) | No. 120) | | | 188) | |
| hsa- | UAGCACCA | GTCGTATCCAGTGCA | 8 | gccgccTAGCA | ROX- | GTGCAGGGT |
| miR- | UUUGAAAU | GGGTCCGAGGTATTC | | CCATTT (SEQ | GAAATCGGT | CCGAGGT |
| 29c-3p | CGGUUA | GCACTGGATACGACT | | ID No. 155) | TAGTCGTATC | (SEQ ID No. |
| | (SEQ ID No. | AACCGAT (SEQ ID | | | CA-MGB (SEQ | 50) |
| | 87) | No. 121) | | | ID No. 189) | |
| hsa- | AAAAGCUG | GTCGTATCCAGTGCA | 8 | ccgccAAAAG | CY5- | GTGCAGGGT |
| miR- | GGUUGAGA | GGGTCCGAGGTATTC | | CTGGGTT | GAGAGGGCA | CCGAGGT |
| 320b- | GGGCAA | GCACTGGATACGACT | | (SEQ ID No. | AGTCGT-MGB | (SEQ ID No. |
| 3p | (SEQ ID No. | TGCCCTC (SEQ ID | | 156) | (SEQ ID No. | 50) |
| | 88) | No. 122) | | | 190) | |
| hsa- | UGAGGUAG | GTCGTATCCAGTGCA | 8 | gcgcgTGAGGT | FAM- | GTGCAGGGT |
| let- | UAGUUUGU | GGGTCCGAGGTATTC | | AGTAGTT | TGTGCTGTTG | CCGAGGT |
| 7i-5p | GCUGUU | GCACTGGATACGAC | | (SEQ ID No. | TCGTATCCA- | (SEQ ID No. |
| | (SEQ ID No. | AACAGCAC (SEQ ID | | 157) | MGB (SEQ ID | 50) |
| | 89) | No. 123) | | | No. 191) | |
| hsa- | UUCAAGUA | GTCGTATCCAGTGCA | 8 | gcgcgcgTTCA | VIC- | GTGCAGGGT |
| miR- | AUUCAGGA | GGGTCCGAGGTATTC | | AGTAATTCA | GGATAGGTG | CCGAGGT |
| 26b-5p | UAGGU | GCACTGGATACGAC | | (SEQ ID No. | TCGTATCCAG | (SEQ ID No. |
| | (SEQ ID No. | ACCTATCC (SEQ ID | | 158) | T-MGB (SEQ | 50) |
| | 90) | No. 124) | | | ID No. 192) | |
| hsa- | AGGGGUGC | GTCGTATCCAGTGCA | 8 | tcgcgAGGGGT | ROX- | GTGCAGGGT |
| miR- | UAUCUGUG | GGGTCCGAGGTATTC | | GCTAT (SEQ | CTGTGATTGA | CCGAGGT |
| 342-5p | AUUGA | GCACTGGATACGACT | | ID No. 159) | GTCGTATCCA- | (SEQ ID No. |
| | (SEQ ID No. | CAATCAC (SEQ ID | | | MGB (SEQ ID | 50) |
| | 91) | No. 125) | | | No. 193) | |
| hsa- | UGGCAGUG | GTCGTATCCAGTGCA | 8 | cgcgTGGCAG | CY5- | GTGCAGGGT |
| miR- | UCUUAGCU | GGGTCCGAGGTATTC | | TGTCT (SEQ | TAGCTGGTTG | CCGAGGT |
| 34a-5p | GGUUGU | GCACTGGATACGAC | | ID No. 160) | TGTCGTAT- | (SEQ ID No. |
| | (SEQ ID No. | ACAACCAG (SEQ ID | | | MGB (SEQ ID | 50) |
| | 92) | No. 126) | | | No. 194) | |
| hsa- | AGGCAGUG | GTCGTATCCAGTGCA | 8 | ttccggAGGCA | FAM- | GTGCAGGGT |
| miR- | UAGUUAGC | GGGTCCGAGGTATTC | | GTGTAGT | TAGCTGATTG | CCGAGGT |
| 34c-5p | UGAUUGC | GCACTGGATACGAC | | (SEQ ID No. | CGTCGTAT- | (SEQ ID No. |
| | (SEQ ID No. | GCAATCAG (SEQ ID | | 161) | MGB (SEQ ID | 50) |
| | 93) | No. 127) | | | No. 195) | |
| hsa- | AGUGGGGA | GTCGTATCCAGTGCA | 8 | gcggcggAGTG | VIC- | GTGCAGGGT |
| miR- | ACCCUUCC | GGGTCCGAGGTATTC | | GGGAA (SEQ | CTTCCATGAG | CCGAGGT |
| 491-5p | AUGAGG | GCACTGGATACGACC | | ID No. 162) | GGTCGTAT- | (SEQ ID No. |
| | (SEQ ID No. | CTCATGG (SEQ ID | | | MGB (SEQ ID | 50) |
| | 94) | No. 128) | | | No. 196) | |
| hsa- | UAUUGCAC | GTCGTATCCAGTGCA | 8 | cggcggTATTG | ROX- | GTGCAGGGT |
| miR- | UUGUCCCG | GGGTCCGAGGTATTC | | CACTTGT | CGGCCTGTGT | CCGAGGT |
| 92a-3p | GCCUGU | GCACTGGATACGAC | | (SEQ ID No. | CGTATCCA- | (SEQ ID No. |
| | (SEQ ID No. | ACAGGCCG (SEQ ID | | 163) | MGB (SEQ ID | 50) |
| | 95) | No. 129) | | | No. 197) | |
| hsa- | CUAUACGA | GTCGTATCCAGTGCA | 8 | gcgcgCTATAC | FAM- | GTGCAGGGT |
| let- | CCUGCUGC | GGGTCCGAGGTATTC | | GACCT (SEQ | CTGCCTTTCT | CCGAGGT |
| 7d-3p | CUUUCU | GCACTGGATACGAC | | ID No. 164) | GTCGTATCCA- | (SEQ ID No. |
| | (SEQ ID No. | AGAAAGGC (SEQ ID | | | MGB (SEQ ID | 50) |
| | 96) | No. 130) | | | No. 198) | |
| hsa- | UUCAAGUA | GTCGTATCCAGTGCA | 8 | cgcgcgcgTTCA | VIC- | GTGCAGGGT |
| miR- | AUCCAGGA | GGGTCCGAGGTATTC | | AGTAATCCA | CCAGGATAG | CCGAGGT |
| 26a-5p | UAGGCU | GCACTGGATACGAC | | (SEQ ID No. | GCTGTCGT- | (SEQ ID No. |
| | (SEQ ID No. | AGCCTATC (SEQ ID | | 165) | MGB (SEQ ID | 50) |
| | 97) | No. 131) | | | No. 199) | |
| hsa- | CAUGCCUU | GTCGTATCCAGTGCA | 8 | cgcgCATGCC | ROX- | GTGCAGGGT |
| miR- | GAGUGUAG | GGGTCCGAGGTATTC | | TTGAGT (SEQ | TAGGACCGT | CCGAGGT |
| 532-5p | GACCGU | GCACTGGATACGAC | | ID No. 166) | GTCGTATCCA- | (SEQ ID No. |
| | (SEQ ID No. | ACGGTCCT (SEQ ID | | | MGB (SEQ ID | 50) |
| | 98) | No. 132) | | | No. 200) | |
| hsa- | GAUCUCAC | GTCGTATCCAGTGCA | 8 | cgcgcgGATCT | CY5- | GTGCAGGGT |
| miR- | UUUGUUGC | GGGTCCGAGGTATTC | | CACTT (SEQ | TGTTGCCCAG | CCGAGGT |
| 1285- | CCAGG | GCACTGGATACGACC | | ID No. 167) | GGTCGT-MGB | (SEQ ID No. |
| 5p | (SEQ ID No. | CTGGGCA (SEQ ID | | | (SEQ ID No. | 50) |
| | 99) | No. 133) | | | 201) | |
| hsa- | UGAGGUAG | GTCGTATCCAGTGCA | 8 | cggcggTGAGG | FAM- | GTGCAGGGT |
| let- | UAGAUUGU | GGGTCCGAGGTATTC | | TAGTAGA | TGTATAGTTG | CCGAGGT |
| 7f-5p | AUAGUU | GCACTGGATACGAC | | (SEQ ID No. | TCGTATCCAG | (SEQ ID No. |
| | (SEQ ID No. | AACTATAC (SEQ ID | | 168) | T-MGB (SEQ | 50) |
| | 100) | No. 134) | | | ID No. 202) | |
| hsa- | UUUUGUGU | GTCGTATCCAGTGCA | 8 | ttcggcggTTTT | VIC- | GTGCAGGGT |
| miR- | CUCCCAUU | GGGTCCGAGGTATTC | | GTGTCT (SEQ | CCATTCCCCA | CCGAGGT |
| 5010- | CCCCAG | GCACTGGATACGACC | | ID No. 169) | GGTCGTAT- | (SEQ ID No. |
| 3p | (SEQ ID No. | TGGGGAA (SEQ ID | | | MGB (SEQ ID | 50) |
| | 101) | No. 135) | | | No. 203) | |
| brain- | GGUCCUGA | GTCGTATCCAGTGCA | 8 | gcggcGGTCCT | ROX- | GTGCAGGGT |
| miR- | CAUCCACG | GGGTCCGAGGTATTC | | GACA (SEQ | TCCACGGAA | CCGAGGT |
| 112 | GAA (SEQ | GCACTGGATACGACT | | ID No. 170) | GTCGTATCCA | (SEQ ID No. |
| | ID No. 102) | TCCGTGG (SEQ ID | | | GT-MGB (SEQ | 50) |
| | | No. 136) | | | ID No. 204) | |
| brain- | AAAAGCUG | GTCGTATCCAGTGCA | 8 | ggcggAAAAG | CY5- | GTGCAGGGT |
| miR- | GGUUGAGA | GGGTCCGAGGTATTC | | CTGGGTT | GAGAGGGCG | CCGAGGT |
| 161 | GGGCGAA | GCACTGGATACGACT | | (SEQ ID No. | AAGTCGTAT- | (SEQ ID No. |
| | (SEQ ID No. | TCGCCCT (SEQ ID | | 171) | MGB (SEQ ID | 50) |
| | 103) | No. 137) | | | No. 205) | |

To summarize the present invention, the sequences of the stem-loop reverse transcription primers, forward/reverse primers, and probes are listed below in Tables 17 and 18.

TABLE 17
Primers and probes used for multiplex RT-qPCR

| | | Stem-loop | | | | | |
|---|---|---|---|---|---|---|---|
| | miRNA | reverse | | | Forward | | Reverse |
| Target | Seq′ | transcription | Stem-loop | Anchor | primer | Probe | primer |
| miRNA | (5′-3′) | primer (5′-3′) | Seq′ (5′-3′) | Seq′ | (5′-3′) | (5′-3′) | (5′-3′) |
| miR-16- | UAGCA | GTCGTATCCAGT | GTCGTATCCA | CGCC | CGCGC | VIC- | TGGCGGTC |
| 5p | GCACG | GCAGGGTCCGAG | GTGCAGGGTC | AATA | GTCTT | GTGCAGGG | GTATCCAG |
| | UAAAU | GTATTCGCACTG | CGAGGTATTC | (SEQ | TGGTT | TCCGAGGT- | TGCGAA |
| | AUUGG | GATACGACCGCC | GCACTGGATA | ID No. | ATCT | BHQ1 (SEQ | (SEQ ID |
| | CG (SEQ | AATA (SEQ ID | CGAC (SEQ ID | 13) | (SEQ ID | ID No. 21) | No. 25) |
| | ID No. 1) | No. 5) | No. 9) | | No. 17) | | |
| miR- | AGGCA | GTCTGTATGGTT | GTCTGTATGG | GCAA | GCGCG | CY5- | CTGATTGC |
| 34c- | GUGUA | GGATAGGGATGT | TTGGATAGGG | TC | AGGCA | TTGGATAG | GTCTGTAT |
| 5p | GUUAG | GAACCAGTCGTG | ATGTGAACCA | (SEQ | GTGTA | GGATGTGA | GGTTGTTC |
| | CUGAU | AACAACCATACA | GTCGTGAACA | ID No. | GTTA | ACCAG- | ACG (SEQ |
| | UGC | GACGCAATC | ACCATACAGA | 14) | (SEQ ID | BHQ3 (SEQ | ID No. 26) |
| | (SEQ ID | (SEQ ID No. 6) | C (SEQ ID | | No. 18) | ID No. 22) | |
| | No. 2) | | No. 10) | | | | |
| miR-9- | AUAAA | GTTGGCTCTGGT | GTTGGCTCTG | ACTTT | TTGCG | ROX- | ACCGAAAG |
| 3p | GCUAG | GCAGGGTCCGAG | GTGCAGGGTC | C (SEQ | CGCAT | GTGCAGGG | TGTTGGCT |
| | AUAAC | GTATTCGCACCA | CGAGGTATTC | ID No. | AAAGC | TCCGAGGT- | CTGGTGC |
| | CGAAA | GAGCCAACACTT | GCACCAGAGC | 15) | TAGAT | BHQ2 (SEQ | (SEQ ID |
| | GU (SEQ | TC (SEQ ID | CAAC (SEQ ID | | (SEQ ID | ID No. 23) | No. 27) |
| | ID No. 3) | No. 7) | No. 11) | | No. 19) | | |
| miR-9- | UCUUU | GGTCGTATGCAA | GGTCGTATGC | TCAT | CGCGC | FAM- | TGTATGAG |
| 5p | GGUUA | AGCAGGGTCCGA | AAAGCAGGGT | ACAG | GTCTT | AGCAGGGT | GTCGTATG |
| | UCUAG | GGTATCCATCGC | CCGAGGTATC | CTA | TGGTT | CCGAGGTA | CAGTGCGA |
| | CUGUA | ACGCATCGCACT | CATCGCACGC | (SEQ | ATCTA | TC-BHQ1 | T (SEQ ID |
| | UGA | GCATACGACCTC | ATCGCACTGC | ID No. | G (SEQ | (SEQ ID | No. 28) |
| | (SEQ ID | ATACAGCTA | ATACGACC | 16) | ID No. | No. 24) | |
| | No. 4) | (SEQ ID No. 8) | (SEQ ID No. | | 20) | | |
| | | | 12) | | | | |

TABLE 17
Stem-loop primer variants with different anchor lengths

| Anchor | Stem-loop primer | Stem-loop primer | Stem-loop primer | Stem-loop primer |
|---|---|---|---|---|
| Seq′ | for | for | for | for |
| Length | miR-16-5p | miR-34c-5p | miR-9-3p | miR-9-5p |
| 11 nt | GTCGTATCCAGTGCA | GTCTGTATGGTTGGAT | GTTGGCTCTGGTGCA | GGTCGTATGCAAAGCA |
| | GGGTCCGAGGTATTC | AGGGATGTGAACCAG | GGGTCCGAGGTATTC | GGGTCCGAGGTATCCA |
| | GCACTGGATACGAC | TCGTGAACAACCATA | GCACCAGAGCCAAC | TCGCACGCATCGCACT |
| | CGCCAATATTT (SEQ | CAGACGCAATCAGCT | ACTTTCGGTTA (SEQ | GCATACGACCTCATAC |
| | ID No. 62) | A (SEQ ID No. 64) | ID No. 66) | AGCTA (SEQ ID No. 8) |
| 8 nt | GTCGTATCCAGTGCA | GTCTGTATGGTTGGAT | GTTGGCTCTGGTGCA | GGTCGTATGCAAAGCA |
| | GGGTCCGAGGTATTC | AGGGATGTGAACCAG | GGGTCCGAGGTATTC | GGGTCCGAGGTATCCA |
| | GCACTGGATACGAC | TCGTGAACAACCATA | GCACCAGAGCCAAC | TCGCACGCATCGCACT |
| | CGCCAATA (SEQ ID | CAGACGCAATCAG | ACTTTCGG (SEQ ID | GCATACGACCTCATAC |
| | No. 5) | (SEQ ID No. 65) | No. 67) | AG (SEQ ID No. 68) |
| 6 nt | GTCGTATCCAGTGCA | GTCTGTATGGTTGGAT | GTTGGCTCTGGTGCA | GGTCGTATGCAAAGCA |
| | GGGTCCGAGGTATTC | AGGGATGTGAACCAG | GGGTCCGAGGTATTC | GGGTCCGAGGTATCCA |
| | GCACTGGATACGAC | TCGTGAACAACCATA | GCACCAGAGCCAAC | TCGCACGCATCGCACT |
| | CGCCAA (SEQ ID | CAGACGCAATC (SEQ | ACTTTC (SEQ ID | GCATACGACCTCATAC |
| | No. 63) | ID No. 6) | No. 7) | (SEQ ID No. 69) |

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

1. A multiplex reverse transcription primer combination for simultaneously quantifying miR-16-5p, miR-34c-5p, miR-9-3p, and miR-9-5p, the multiplex reverse transcription primer combination comprising:
a first stem-loop reverse transcription primer comprising a nucleic acid sequence of SEQ ID No. 5;
a second stem-loop reverse transcription primer comprising a nucleic acid sequence of SEQ ID No. 6;
a third stem-loop reverse transcription primer comprising a nucleic acid sequence of SEQ ID No. 7; and
a fourth stem-loop reverse transcription primer comprising a nucleic acid sequence of SEQ ID No. 8.

2. A multiplex reverse transcription primer combination for simultaneously quantifying a plurality of target miRNAs, the multiplex reverse transcription primer combination comprising:
a plurality of stem-loop reverse transcription primers; wherein each of the plurality of stem-loop reverse transcription primers has a stem-loop sequence forming a stem-loop structure and an anchor sequence complimentary to a unique 3′ sequence of one of the plurality of target miRNAs;
wherein the lengths of the anchor sequences of at least two of the stem-loop reverse transcription primers are different from each other;
wherein the plurality of stem-loop reverse transcription primers comprises a first stem-loop reverse transcription primer having a first stem-loop sequence and a first anchor sequence; and
a second stem-loop reverse transcription primer having a second stem-loop sequence and a second anchor sequence,
wherein each of the first and second stem-loop sequences is selected from the group consisting of SEQ ID Nos. 9-12; and the first and second stem-loop sequences are different from each other; and
wherein the length of each of the first and second anchor sequences is selected from one of 4 nt, 6 nt, 8 nt, and 11 nt.

3. The multiplex reverse transcription primer combination according to claim 2, wherein each of the plurality of stem-loop reverse transcription primers has a length of about 40-65 nt.

4. The multiplex reverse transcription primer combination according to claim 2, wherein the anchor sequence of each of the plurality of stem-loop reverse transcription primers has a length of about 3-12 nt.

5. (canceled)

6. The multiplex reverse transcription primer combination according to claim 2, wherein the first anchor sequence of the first stem-loop reverse transcription primer is complimentary to a unique 3′ sequence of a first target miRNA of the plurality of target miRNAs and the second anchor sequence of the second stem-loop reverse transcription primer is complimentary to a unique 3′ sequence of a second target miRNA of the plurality of target miRNAs.

7. The multiplex reverse transcription primer combination according to claim 2, wherein the plurality of stem-loop reverse transcription primers further comprises a third stem-loop reverse transcription primer having a third stem-loop sequence and a third anchor sequence,
wherein the third stem-loop sequence is selected from the group consisting of SEQ ID Nos. 9-12; and the third stem-loop sequence is different from the first and second stem-loop sequences;
wherein the length of the third anchor sequence is selected from one of 4 nt, 6 nt, 8 nt, and 11 nt; and
wherein the third anchor sequence is complimentary to a unique 3′ sequence of a third target miRNA of the plurality of target miRNAs.

8. The multiplex reverse transcription primer combination according to claim 7, wherein the plurality of stem-loop reverse transcription primers further comprises a fourth stem-loop reverse transcription primer having a fourth stem-loop sequence and a fourth anchor sequence,
wherein the fourth stem-loop sequence is selected from the group consisting of SEQ ID Nos. 9-12; and the fourth stem-loop sequence is different from the first, second, and third stem-loop sequences;
wherein the length of the fourth anchor sequence is selected from one of 4 nt, 6 nt, 8 nt, and 11 nt; and
wherein the fourth anchor sequence is complimentary to a unique 3′ sequence of a fourth target miRNA of the plurality of target miRNAs.

9. The multiplex reverse transcription primer combination according to claim 8, wherein the multiplex reverse transcription primer combination has multi-specificity such that the first stem-loop reverse transcription primer effectively and only reverse transcribes the first target miRNA, the second stem-loop reverse transcription primer effectively and only reverse transcribes the second target miRNA, the third stem-loop reverse transcription primer effectively and only reverse transcribes the third target miRNA, and the fourth stem-loop reverse transcription primer effectively and only reverse transcribes the fourth target miRNA.

10. The multiplex reverse transcription primer combination according to claim 9, wherein each of the first, second, third and fourth target miRNAs is selected from the group consisting of miR-16-5p, miR-34c-5p, miR-9-3p, and miR-9-5p,
wherein each of the first, second, third and fourth target miRNAs is different from the others.

11. The multiplex reverse transcription primer combination according to claim 8, wherein the first stem-loop reverse transcription primer comprises a nucleic acid sequence of SEQ ID No. 5, the second stem-loop reverse transcription primer comprises a nucleic acid sequence of SEQ ID No. 6, the third stem-loop reverse transcription primer comprises a nucleic acid sequence of SEQ ID No. 7, and the fourth stem-loop reverse transcription primer comprises a nucleic acid sequence of SEQ ID No. 8.

12. A kit for simultaneously quantifying expression level of a plurality of target miRNAs, the kit comprising:
a first stem-loop reverse transcription primer and a second stem-loop reverse transcription primer, wherein the first stem-loop reverse transcription primer has a first stem-loop sequence and a first anchor sequence, and the second stem-loop reverse transcription primer has a second stem-loop sequence and a second anchor sequence,
wherein each of the first and second stem-loop sequences is selected from the group consisting of SEQ ID Nos. 9-12; and the first and second stem-loop sequences are different from each other,
wherein the length of each of the first and second anchor sequences is selected from one of 4 nt, 6 nt, 8 nt, and 11 nt;
first and second forward primers, wherein each of the first and second forward primers comprises a nucleic acid sequence selected from the group consisting of SEQ ID No. 17-20, and the first forward primer is different from the second forward primer;
first and second reverse primers; wherein each of the first and second reverse primers comprises a nucleic acid sequence selected from the group consisting of SEQ ID No. 25-28, and the first reverse primer is different from the second reverse primer; and
first and second probes, wherein the first probe comprises a first probe sequence, a first fluorescent reporter group, and a first quencher group; and the second probe comprises a second probe sequence, a second fluorescent reporter group, and a second quencher group,
wherein each of the first and second probe sequences comprises a nucleic acid sequence selected from the group consisting of SEQ ID No. 21-24.

13. The kit according to claim 12, wherein
each of the first and second fluorescent reporter groups is selected from the group consisting of VIC, CY5, ROX, and FAM; and the first fluorescent reporter group is different from the second fluorescent reporter group.

14. The kit according to claim 13, wherein the first anchor sequence is complimentary to a 3′ sequence of a first target miRNA of the plurality of target miRNAs, and the second anchor sequence is complimentary to a unique 3′ sequence of a second target miRNA of the plurality of target miRNAs.

15. The kit according to claim 12 further comprising:
a third stem-loop reverse transcription primer, wherein the third stem-loop reverse transcription primer has a third stem-loop sequence and a third anchor sequence,
wherein the third stem-loop sequence is selected from the group consisting of SEQ ID Nos. 9-12; and the third stem-loop sequence is different from the first and second stem-loop sequences, and wherein the length of the third anchor sequence is selected from one of 4 nt, 6 nt, 8 nt, and 11 nt;
a third forward primer, wherein the third forward primer comprises a nucleic acid sequence selected from the group consisting of SEQ ID Nos. 17-20; and the third forward primer is different from the first and second forward primers;
a third reverse primer, wherein the third reverse primer comprises a nucleic acid sequence selected from the group consisting of SEQ ID Nos. 25-28; and the third reverse primer is different from the first and second reverse primers; and
a third probe, wherein the third probe comprises a third probe sequence, a third fluorescent reporter group, and a third quencher group; wherein the third probe sequence comprises a nucleic acid sequence selected from the group consisting of SEQ ID Nos. 21-24.

16. The kit according to claim 15, wherein
the third fluorescent reporter group is selected from the group consisting of VIC, CY5, ROX, and FAM; and the third fluorescent reporter group is different from the first and second fluorescent reporter groups.

17. The kit according to claim 16 further comprising:
a fourth stem-loop reverse transcription primer, wherein the fourth stem-loop reverse transcription primer has a fourth stem-loop sequence and a fourth anchor sequence,
wherein the fourth stem-loop sequence is selected from the group consisting of SEQ ID Nos. 9-12; and the fourth stem-loop sequence is different from the first, second, and third stem-loop sequences, wherein the length of the fourth anchor sequence is selected from one of 4 nt, 6 nt, 8 nt, and 11 nt;
a fourth forward primer, wherein the fourth forward primer comprises a nucleic acid sequence selected from the group consisting of SEQ ID Nos. 17-20; and the fourth forward primer is different from the first, second, and third forward primers;
a fourth reverse primer, wherein the fourth reverse primer comprises a nucleic acid sequence selected from the group consisting of SEQ ID Nos. 25-28; and the fourth reverse primer is different from the first, second, and third reverse primers; and
a fourth probe, wherein the fourth probe comprises a fourth probe sequence, a fourth fluorescent reporter group, and a fourth quencher group; wherein the fourth probe sequence comprises a nucleic acid sequence selected from the group consisting of SEQ ID Nos. 21-24.

18. The kit according to claim 17, wherein
the fourth fluorescent reporter group is selected from the group consisting of VIC, CY5, ROX, and FAM; and the fourth fluorescent reporter group is different from the first, second, and third fluorescent reporter groups.

19. A kit for simultaneously quantifying expression level of a plurality of target miRNAs, the kit comprising:
a multiplex reverse transcription primer combination having a first stem-loop reverse transcription primer comprising a nucleic acid sequence of SEQ ID No. 5, a second stem-loop reverse transcription primer comprising a nucleic acid sequence of SEQ ID No. 6, a third stem-loop reverse transcription primer comprising a nucleic acid sequence of SEQ ID No. 7, and a fourth stem-loop reverse transcription primer comprising a nucleic acid sequence of SEQ ID No. 8;
a forward primer combination having a first forward primer comprising a nucleic acid sequence of SEQ ID No. 17, a second forward primer comprising a nucleic acid sequence of SEQ ID No. 18, a third forward primer comprising a nucleic acid sequence of SEQ ID No: 19; and a fourth forward primer comprising a nucleic acid sequence of SEQ ID No. 20;
a reverse primer combination having a first reverse primer comprising a nucleic acid sequence of SEQ ID No. 25, a second reverse primer comprising a nucleic acid sequence of SEQ ID No. 26, a third reverse primer comprising a nucleic acid sequence of SEQ ID No. 27; and a fourth reverse primer comprising a nucleic acid sequence of SEQ ID No. 28; and
a probe combination having a first probe comprising a nucleic acid sequence of SEQ ID No. 21 and a first fluorescent reporter group of VIC, a second probe comprising a nucleic acid sequence of SEQ ID No. 22 and a second fluorescent reporter group of CY5, a third probe comprising a nucleic acid sequence of SEQ ID No. 23 and a third fluorescent reporter group of ROX, a fourth probe comprising a nucleic acid sequence of SEQ ID No. 24 and a fourth fluorescent reporter group of FAM.

20. A method of simultaneously quantifying miR-16-5p, miR-34c-5p, miR-9-3p, and miR-9-5p, the method comprising performing multiplex RT-qPCR using the multiplex reverse transcription primer combination of claim 2.

21. A method for using quantification results of miR-16-5p, miR-34c-5p, miR-9-3p, and miR-9-5p for determining a medical condition of a living subject, the method comprising:
performing multiplex RT-qPCR using the kit of claim 19;
obtaining quantification results of miR-16-5p, miR-34c-5p, miR-9-3p, and miR-9-5p from the multiplex RT-qPCR; and
using the quantification results for determining a medical condition of the living subject,
wherein the medical condition of the living subject is Alzheimer's Disease.


SEQUENCE LISTING (264 sequences)

SEQ ID NO: 1 - RNA, 22 nt, Homo sapiens
  source 1..22: mol_type = other RNA
tagcagcacgtaaatattggcg

SEQ ID NO: 2 - RNA, 23 nt, Homo sapiens
  source 1..23: mol_type = other RNA
aggcagtgtagttagctgattgc

SEQ ID NO: 3 - RNA, 22 nt, Homo sapiens
  source 1..22: mol_type = other RNA
ataaagctagataaccgaaagt

SEQ ID NO: 4 - RNA, 23 nt, Homo sapiens
  source 1..23: mol_type = other RNA
tctttggttatctagctgtatga

SEQ ID NO: 5 - DNA, 52 bp, synthetic construct
  source 1..52: mol_type = other DNA
gtcgtatccagtgcagggtccgaggtattcgcactggatacgaccgccaata

SEQ ID NO: 6 - DNA, 57 bp, synthetic construct
  source 1..57: mol_type = other DNA
gtctgtatggttggatagggatgtgaaccagtcgtgaacaaccatacagacgcaatc

SEQ ID NO: 7 - DNA, 50 bp, synthetic construct
  source 1..50: mol_type = other DNA
gttggctctggtgcagggtccgaggtattcgcaccagagccaacactttc

SEQ ID NO: 8 - DNA, 69 bp, synthetic construct
  source 1..69: mol_type = other DNA
ggtcgtatgcaaagcagggtccgaggtatccatcgcacgcatcgcactgcatacgacctcatacagcta

SEQ ID NO: 9 - DNA, 44 bp, synthetic construct
  source 1..44: mol_type = other DNA
gtcgtatccagtgcagggtccgaggtattcgcactggatacgac

SEQ ID NO: 10 - DNA, 51 bp, synthetic construct
  source 1..51: mol_type = other DNA
gtctgtatggttggatagggatgtgaaccagtcgtgaacaaccatacagac

SEQ ID NO: 11 - DNA, 44 bp, synthetic construct
  source 1..44: mol_type = other DNA
gttggctctggtgcagggtccgaggtattcgcaccagagccaac

SEQ ID NO: 12 - DNA, 58 bp, synthetic construct
  source 1..58: mol_type = other DNA
ggtcgtatgcaaagcagggtccgaggtatccatcgcacgcatcgcactgcatacgacc

SEQ ID NO: 13-15 - skipped (3 entries)

SEQ ID NO: 16 - DNA, 11 bp, synthetic construct
  source 1..11: mol_type = other DNA
tcatacagcta

SEQ ID NO: 17 - DNA, 19 bp, synthetic construct
  source 1..19: mol_type = other DNA
cgcgcgtctttggttatct

SEQ ID NO: 18 - DNA, 19 bp, synthetic construct
  source 1..19: mol_type = other DNA
gcgcgaggcagtgtagtta

SEQ ID NO: 19 - DNA, 20 bp, synthetic construct
  source 1..20: mol_type = other DNA
ttgcgcgcataaagctagat

SEQ ID NO: 20 - DNA, 21 bp, synthetic construct
  source 1..21: mol_type = other DNA
cgcgcgtctttggttatctag

SEQ ID NO: 21 - DNA, 16 bp, synthetic construct
  source 1..16: mol_type = other DNA
gtgcagggtccgaggt

SEQ ID NO: 22 - DNA, 21 bp, synthetic construct
  source 1..21: mol_type = other DNA
ttggatagggatgtgaaccag

SEQ ID NO: 23 - DNA, 16 bp, synthetic construct
  source 1..16: mol_type = other DNA
gtgcagggtccgaggt

SEQ ID NO: 24 - DNA, 18 bp, synthetic construct
  source 1..18: mol_type = other DNA
agcagggtccgaggtatc

SEQ ID NO: 25 - DNA, 22 bp, synthetic construct
  source 1..22: mol_type = other DNA
tggcggtcgtatccagtgcgaa

SEQ ID NO: 26 - DNA, 27 bp, synthetic construct
  source 1..27: mol_type = other DNA
ctgattgcgtctgtatggttgttcacg

SEQ ID NO: 27 - DNA, 23 bp, synthetic construct
  source 1..23: mol_type = other DNA
accgaaagtgttggctctggtgc

SEQ ID NO: 28 - DNA, 25 bp, synthetic construct
  source 1..25: mol_type = other DNA
tgtatgaggtcgtatgcagtgcgat

SEQ ID NO: 29 - RNA, 22 nt, Homo sapiens
  source 1..22: mol_type = other RNA
ctgtgcgtgtgacagcggctga

SEQ ID NO: 30 - RNA, 22 nt, Homo sapiens
  source 1..22: mol_type = other RNA
tcgtaccgtgagtaataatgcg

SEQ ID NO: 31 - RNA, 22 nt, Homo sapiens
  source 1..22: mol_type = other RNA
tccttcattccaccggagtctg

SEQ ID NO: 32 - RNA, 22 nt, Homo sapiens
  source 1..22: mol_type = other RNA
tcctgtactgagctgccccgag

SEQ ID NO: 33 - DNA, 52 bp, synthetic construct
  source 1..52: mol_type = other DNA
gtcgtatccagtgcagggtccgaggtattcgcactggatacgactcagccgc

SEQ ID NO: 34 - DNA, 50 bp, synthetic construct
  source 1..50: mol_type = other DNA
gtcgtatccagtgcagggtccgaggtattcgcactggatacgaccgcatt

SEQ ID NO: 35 - DNA, 50 bp, synthetic construct
  source 1..50: mol_type = other DNA
gtcgtatccagtgcagggtccgaggtattcgcactggatacgaccagact

SEQ ID NO: 36 - DNA, 52 bp, synthetic construct
  source 1..52: mol_type = other DNA
gtcgtatccagtgcagggtccgaggtattcgcactggatacgacctcggggc

SEQ ID NO: 37 - DNA, 44 bp, synthetic construct; identical to SEQ ID NO: 9
  source 1..44: mol_type = other DNA

SEQ ID NO: 38-41 - skipped (4 entries)

SEQ ID NO: 42 - DNA, 17 bp, synthetic construct
  source 1..17: mol_type = other DNA
atgcctgtgcgtgtgac

SEQ ID NO: 43 - DNA, 16 bp, synthetic construct
  source 1..16: mol_type = other DNA
ttcgcgctcgtaccgt

SEQ ID NO: 44 - DNA, 18 bp, synthetic construct
  source 1..18: mol_type = other DNA
ttgccgcctccttcattc

SEQ ID NO: 45 - DNA, 17 bp, synthetic construct
  source 1..17: mol_type = other DNA
gccgcctcctgtactga

SEQ ID NO: 46 - DNA, 16 bp, synthetic construct
  source 1..16: mol_type = other DNA
agcggctgagtcgtat

SEQ ID NO: 47 - DNA, 21 bp, synthetic construct
  source 1..21: mol_type = other DNA
agtaataatgcggtcgtatcc

SEQ ID NO: 48 - DNA, 18 bp, synthetic construct
  source 1..18: mol_type = other DNA
ccggagtctggtcgtatc

SEQ ID NO: 49 - DNA, 17 bp, synthetic construct
  source 1..17: mol_type = other DNA
tgccccgaggtcgtatc

SEQ ID NO: 50 - DNA, 16 bp, synthetic construct
  source 1..16: mol_type = other DNA
gtgcagggtccgaggt

SEQ ID NO: 51 - DNA, 55 bp, synthetic construct
  source 1..55: mol_type = other DNA
gtcgtatccagtgcagggtccgaggtattcgcactggatacgactcagccgctgt

SEQ ID NO: 52 - DNA, 50 bp, synthetic construct
  source 1..50: mol_type = other DNA
gtcgtatccagtgcagggtccgaggtattcgcactggatacgactcagcc

SEQ ID NO: 53 - DNA, 48 bp, synthetic construct
  source 1..48: mol_type = other DNA
gtcgtatccagtgcagggtccgaggtattcgcactggatacgactcag

SEQ ID NO: 54 - DNA, 55 bp, synthetic construct
  source 1..55: mol_type = other DNA
gtcgtatccagtgcagggtccgaggtattcgcactggatacgaccgcattattac

SEQ ID NO: 55 - DNA, 48 bp, synthetic construct
  source 1..48: mol_type = other DNA
gtcgtatccagtgcagggtccgaggtattcgcactggatacgaccgca

SEQ ID NO: 56 - DNA, 55 bp, synthetic construct
  source 1..55: mol_type = other DNA
gtcgtatccagtgcagggtccgaggtattcgcactggatacgaccagactccggt

SEQ ID NO: 57 - DNA, 52 bp, synthetic construct
  source 1..52: mol_type = other DNA
gtcgtatccagtgcagggtccgaggtattcgcactggatacgaccagactcc

SEQ ID NO: 58 - DNA, 48 bp, synthetic construct
  source 1..48: mol_type = other DNA
gtcgtatccagtgcagggtccgaggtattcgcactggatacgaccaga

SEQ ID NO: 59 - DNA, 55 bp, synthetic construct
  source 1..55: mol_type = other DNA
gtcgtatccagtgcagggtccgaggtattcgcactggatacgacctcggggcagc

SEQ ID NO: 60 - DNA, 50 bp, synthetic construct
  source 1..50: mol_type = other DNA
gtcgtatccagtgcagggtccgaggtattcgcactggatacgacctcggg

SEQ ID NO: 61 - DNA, 48 bp, synthetic construct
  source 1..48: mol_type = other DNA
gtcgtatccagtgcagggtccgaggtattcgcactggatacgacctcg

SEQ ID NO: 62 - DNA, 55 bp, synthetic construct
  source 1..55: mol_type = other DNA
gtcgtatccagtgcagggtccgaggtattcgcactggatacgaccgccaatattt

SEQ ID NO: 63 - DNA, 50 bp, synthetic construct
  source 1..50: mol_type = other DNA
gtcgtatccagtgcagggtccgaggtattcgcactggatacgaccgccaa

SEQ ID NO: 64 - DNA, 62 bp, synthetic construct
  source 1..62: mol_type = other DNA
gtctgtatggttggatagggatgtgaaccagtcgtgaacaaccatacagacgcaatcagcta

SEQ ID NO: 65 - DNA, 59 bp, synthetic construct
  source 1..59: mol_type = other DNA
gtctgtatggttggatagggatgtgaaccagtcgtgaacaaccatacagacgcaatcag

SEQ ID NO: 66 - DNA, 55 bp, synthetic construct
  source 1..55: mol_type = other DNA
gttggctctggtgcagggtccgaggtattcgcaccagagccaacactttcggtta

SEQ ID NO: 67 - DNA, 52 bp, synthetic construct
  source 1..52: mol_type = other DNA
gttggctctggtgcagggtccgaggtattcgcaccagagccaacactttcgg

SEQ ID NO: 68 - DNA, 66 bp, synthetic construct
  source 1..66: mol_type = other DNA
ggtcgtatgcaaagcagggtccgaggtatccatcgcacgcatcgcactgcatacgacctcatacag

SEQ ID NO: 69 - DNA, 64 bp, synthetic construct
  source 1..64: mol_type = other DNA
ggtcgtatgcaaagcagggtccgaggtatccatcgcacgcatcgcactgcatacgacctcatac

SEQ ID NO: 70 - RNA, 23 nt, Homo sapiens
  source 1..23: mol_type = other RNA
tgtagtgtttcctactttatgga

SEQ ID NO: 71 - RNA, 22 nt, Homo sapiens
  source 1..22: mol_type = other RNA
taacagtctacagccatggtcg

SEQ ID NO: 72 - RNA, 22 nt, Homo sapiens
  source 1..22: mol_type = other RNA
tgagaactgaattccatgggtt

SEQ ID NO: 73 - RNA, 22 nt, Homo sapiens
  source 1..22: mol_type = other RNA
aaaagctgggttgagagggcga

SEQ ID NO: 74 - RNA, 22 nt, Homo sapiens
  source 1..22: mol_type = other RNA
gcctgctggggtggaacctggt

SEQ ID NO: 75 - RNA, 21 nt, Homo sapiens
  source 1..21: mol_type = other RNA
gcagtccatgggcatatacac

SEQ ID NO: 76 - RNA, 22 nt, Homo sapiens
  source 1..22: mol_type = other RNA
tcctgtactgagctgccccgag

SEQ ID NO: 77 - RNA, 23 nt, Homo sapiens
  source 1..23: mol_type = other RNA
aaaagtgcttacagtgcaggtag

SEQ ID NO: 78 - RNA, 23 nt, Homo sapiens
  source 1..23: mol_type = other RNA
agcagcattgtacagggctatga

SEQ ID NO: 79 - RNA, 22 nt, Homo sapiens
  source 1..22: mol_type = other RNA
tcgtaccgtgagtaataatgcg

SEQ ID NO: 80 - RNA, 22 nt, Homo sapiens
  source 1..22: mol_type = other RNA
tagcagcacatcatggtttaca

SEQ ID NO: 81 - RNA, 23 nt, Homo sapiens
  source 1..23: mol_type = other RNA
tctcacacagaaatcgcacccgt

SEQ ID NO: 82 - RNA, 23 nt, Homo sapiens
  source 1..23: mol_type = other RNA
agcagcattgtacagggctatca

SEQ ID NO: 83 - RNA, 22 nt, Homo sapiens
  source 1..22: mol_type = other RNA
aacattcaacctgtcggtgagt

SEQ ID NO: 84 - RNA, 22 nt, Homo sapiens
  source 1..22: mol_type = other RNA
tagcttatcagactgatgttga

SEQ ID NO: 85 - RNA, 23 nt, Homo sapiens
  source 1..23: mol_type = other RNA
cgcatcccctagggcattggtgt

SEQ ID NO: 86 - RNA, 21 nt, Homo sapiens
  source 1..21: mol_type = other RNA
ctagactgaagctccttgagg

SEQ ID NO: 87 - RNA, 22 nt, Homo sapiens
  source 1..22: mol_type = other RNA
tagcaccatttgaaatcggtta

SEQ ID NO: 88 - RNA, 22 nt, Homo sapiens
  source 1..22: mol_type = other RNA
aaaagctgggttgagagggcaa

SEQ ID NO: 89 - RNA, 22 nt, Homo sapiens
  source 1..22: mol_type = other RNA
tgaggtagtagtttgtgctgtt

SEQ ID NO: 90 - RNA, 21 nt, Homo sapiens
  source 1..21: mol_type = other RNA
ttcaagtaattcaggataggt

SEQ ID NO: 91 - RNA, 21 nt, Homo sapiens
  source 1..21: mol_type = other RNA
aggggtgctatctgtgattga

SEQ ID NO: 92 - RNA, 22 nt, Homo sapiens
  source 1..22: mol_type = other RNA
tggcagtgtcttagctggttgt

SEQ ID NO: 93 - RNA, 23 nt, Homo sapiens
  source 1..23: mol_type = other RNA
aggcagtgtagttagctgattgc

SEQ ID NO: 94 - RNA, 22 nt, Homo sapiens
  source 1..22: mol_type = other RNA
agtggggaacccttccatgagg

SEQ ID NO: 95 - RNA, 22 nt, Homo sapiens
  source 1..22: mol_type = other RNA
tattgcacttgtcccggcctgt

SEQ ID NO: 96 - RNA, 22 nt, Homo sapiens
  source 1..22: mol_type = other RNA
ctatacgacctgctgcctttct

SEQ ID NO: 97 - RNA, 22 nt, Homo sapiens
  source 1..22: mol_type = other RNA
ttcaagtaatccaggataggct

SEQ ID NO: 98 - RNA, 22 nt, Homo sapiens
  source 1..22: mol_type = other RNA
catgccttgagtgtaggaccgt

SEQ ID NO: 99 - RNA, 21 nt, Homo sapiens
  source 1..21: mol_type = other RNA
gatctcactttgttgcccagg

SEQ ID NO: 100 - RNA, 22 nt, Homo sapiens
  source 1..22: mol_type = other RNA
tgaggtagtagattgtatagtt

SEQ ID NO: 101 - RNA, 22 nt, Homo sapiens
  source 1..22: mol_type = other RNA
ttttgtgtctcccattccccag

SEQ ID NO: 102 - RNA, 19 nt, Homo sapiens
  source 1..19: mol_type = other RNA
ggtcctgacatccacggaa

SEQ ID NO: 103 - RNA, 23 nt, Homo sapiens
  source 1..23: mol_type = other RNA
aaaagctgggttgagagggcgaa

SEQ ID NO: 104 - DNA, 52 bp, synthetic construct
  source 1..52: mol_type = other DNA
gtcgtatccagtgcagggtccgaggtattcgcactggatacgactccataaa

SEQ ID NO: 105 - DNA, 52 bp, synthetic construct
  source 1..52: mol_type = other DNA
gtcgtatccagtgcagggtccgaggtattcgcactggatacgaccgaccatg

SEQ ID NO: 106 - DNA, 52 bp, synthetic construct
  source 1..52: mol_type = other DNA
gtcgtatccagtgcagggtccgaggtattcgcactggatacgacaacccatg

SEQ ID NO: 107 - DNA, 52 bp, synthetic construct
  source 1..52: mol_type = other DNA
gtcgtatccagtgcagggtccgaggtattcgcactggatacgactcgccctc

SEQ ID NO: 108 - DNA, 52 bp, synthetic construct
  source 1..52: mol_type = other DNA
gtcgtatccagtgcagggtccgaggtattcgcactggatacgacaccaggtt

SEQ ID NO: 109 - DNA, 52 bp, synthetic construct
  source 1..52: mol_type = other DNA
gtcgtatccagtgcagggtccgaggtattcgcactggatacgacgtgtatat

SEQ ID NO: 110 - DNA, 52 bp, synthetic construct; identical to SEQ ID NO: 36
  source 1..52: mol_type = other DNA

SEQ ID NO: 111 - DNA, 52 bp, synthetic construct
  source 1..52: mol_type = other DNA
gtcgtatccagtgcagggtccgaggtattcgcactggatacgacctacctgc

SEQ ID NO: 112 - DNA, 52 bp, synthetic construct
  source 1..52: mol_type = other DNA
gtcgtatccagtgcagggtccgaggtattcgcactggatacgactcatagcc

SEQ ID NO: 113 - DNA, 50 bp, synthetic construct; identical to SEQ ID NO: 34
  source 1..50: mol_type = other DNA

SEQ ID NO: 114 - DNA, 52 bp, synthetic construct
  source 1..52: mol_type = other DNA
gtcgtatccagtgcagggtccgaggtattcgcactggatacgactgtaaacc

SEQ ID NO: 115 - DNA, 52 bp, synthetic construct
  source 1..52: mol_type = other DNA
gtcgtatccagtgcagggtccgaggtattcgcactggatacgacacgggtgc

SEQ ID NO: 116 - DNA, 52 bp, synthetic construct
  source 1..52: mol_type = other DNA
gtcgtatccagtgcagggtccgaggtattcgcactggatacgactgatagcc

SEQ ID NO: 117 - DNA, 52 bp, synthetic construct
  source 1..52: mol_type = other DNA
gtcgtatccagtgcagggtccgaggtattcgcactggatacgacactcaccg

SEQ ID NO: 118 - DNA, 52 bp, synthetic construct
  source 1..52: mol_type = other DNA
gtcgtatccagtgcagggtccgaggtattcgcactggatacgactcaacatc

SEQ ID NO: 119 - DNA, 52 bp, synthetic construct
  source 1..52: mol_type = other DNA
gtcgtatccagtgcagggtccgaggtattcgcactggatacgacacaccaat

SEQ ID NO: 120 - DNA, 52 bp, synthetic construct
  source 1..52: mol_type = other DNA
gtcgtatccagtgcagggtccgaggtattcgcactggatacgaccctcaagg

SEQ ID NO: 121 - DNA, 52 bp, synthetic construct
  source 1..52: mol_type = other DNA
gtcgtatccagtgcagggtccgaggtattcgcactggatacgactaaccgat

SEQ ID NO: 122 - DNA, 52 bp, synthetic construct
  source 1..52: mol_type = other DNA
gtcgtatccagtgcagggtccgaggtattcgcactggatacgacttgccctc

SEQ ID NO: 123 - DNA, 52 bp, synthetic construct
  source 1..52: mol_type = other DNA
gtcgtatccagtgcagggtccgaggtattcgcactggatacgacaacagcac

SEQ ID NO: 124 - DNA, 52 bp, synthetic construct
  source 1..52: mol_type = other DNA
gtcgtatccagtgcagggtccgaggtattcgcactggatacgacacctatcc

SEQ ID NO: 125 - DNA, 52 bp, synthetic construct
  source 1..52: mol_type = other DNA
gtcgtatccagtgcagggtccgaggtattcgcactggatacgactcaatcac

SEQ ID NO: 126 - DNA, 52 bp, synthetic construct
  source 1..52: mol_type = other DNA
gtcgtatccagtgcagggtccgaggtattcgcactggatacgacacaaccag

SEQ ID NO: 127 - DNA, 52 bp, synthetic construct
  source 1..52: mol_type = other DNA
gtcgtatccagtgcagggtccgaggtattcgcactggatacgacgcaatcag

SEQ ID NO: 128 - DNA, 52 bp, synthetic construct
  source 1..52: mol_type = other DNA
gtcgtatccagtgcagggtccgaggtattcgcactggatacgaccctcatgg

SEQ ID NO: 129 - DNA, 52 bp, synthetic construct
  source 1..52: mol_type = other DNA
gtcgtatccagtgcagggtccgaggtattcgcactggatacgacacaggccg

SEQ ID NO: 130 - DNA, 52 bp, synthetic construct
  source 1..52: mol_type = other DNA
gtcgtatccagtgcagggtccgaggtattcgcactggatacgacagaaaggc

SEQ ID NO: 131 - DNA, 52 bp, synthetic construct
  source 1..52: mol_type = other DNA
gtcgtatccagtgcagggtccgaggtattcgcactggatacgacagcctatc

SEQ ID NO: 132 - DNA, 52 bp, synthetic construct
  source 1..52: mol_type = other DNA
gtcgtatccagtgcagggtccgaggtattcgcactggatacgacacggtcct

SEQ ID NO: 133 - DNA, 52 bp, synthetic construct
  source 1..52: mol_type = other DNA
gtcgtatccagtgcagggtccgaggtattcgcactggatacgaccctgggca

SEQ ID NO: 134 - DNA, 52 bp, synthetic construct
  source 1..52: mol_type = other DNA
gtcgtatccagtgcagggtccgaggtattcgcactggatacgacaactatac

SEQ ID NO: 135 - DNA, 52 bp, synthetic construct
  source 1..52: mol_type = other DNA
gtcgtatccagtgcagggtccgaggtattcgcactggatacgacctggggaa

SEQ ID NO: 136 - DNA, 52 bp, synthetic construct
  source 1..52: mol_type = other DNA
gtcgtatccagtgcagggtccgaggtattcgcactggatacgacttccgtgg

SEQ ID NO: 137 - DNA, 52 bp, synthetic construct
  source 1..52: mol_type = other DNA
gtcgtatccagtgcagggtccgaggtattcgcactggatacgacttcgccct

SEQ ID NO: 138 - DNA, 18 bp, synthetic construct
  source 1..18: mol_type = other DNA
gccgcctgtagtgtttcc

SEQ ID NO: 139 - DNA, 18 bp, synthetic construct
  source 1..18: mol_type = other DNA
cgccgcctaacagtctac

SEQ ID NO: 140 - DNA, 18 bp, synthetic construct
  source 1..18: mol_type = other DNA
cgcgcgtgagaactgaat

SEQ ID NO: 141 - DNA, 16 bp, synthetic construct
  source 1..16: mol_type = other DNA
ccgcgaaaagctgggt

SEQ ID NO: 142 - DNA, 15 bp, synthetic construct
  source 1..15: mol_type = other DNA
ttcgcctgctggggt

SEQ ID NO: 143 - DNA, 15 bp, synthetic construct
  source 1..15: mol_type = other DNA
tccgccgcagtccat

SEQ ID NO: 144 - DNA, 17 bp, synthetic construct
  source 1..17: mol_type = other DNA
gccgcctcctgtactga

SEQ ID NO: 145 - DNA, 19 bp, synthetic construct
  source 1..19: mol_type = other DNA
gccgccaaaagtgcttaca

SEQ ID NO: 146 - DNA, 16 bp, synthetic construct
  source 1..16: mol_type = other DNA
cgcgcagcagcattgt

SEQ ID NO: 147 - DNA, 16 bp, synthetic construct
  source 1..16: mol_type = other DNA
ttcgcgctcgtaccgt

SEQ ID NO: 148 - DNA, 16 bp, synthetic construct
  source 1..16: mol_type = other DNA
cgcgctagcagcacat

SEQ ID NO: 149 - DNA, 17 bp, synthetic construct
  source 1..17: mol_type = other DNA
gcgcgctctcacacaga

SEQ ID NO: 150 - DNA, 18 bp, synthetic construct
  source 1..18: mol_type = other DNA
cgcgcgagcagcattgta

SEQ ID NO: 151 - DNA, 18 bp, synthetic construct
  source 1..18: mol_type = other DNA
cgcgcgaacattcaacct

SEQ ID NO: 152 - DNA, 19 bp, synthetic construct
  source 1..19: mol_type = other DNA
gcgcgcgtagcttatcaga

SEQ ID NO: 153 - DNA, 15 bp, synthetic construct
  source 1..15: mol_type = other DNA
gcggcgcatccccta

SEQ ID NO: 154 - DNA, 17 bp, synthetic construct
  source 1..17: mol_type = other DNA
cgccgccctagactgaa

SEQ ID NO: 155 - DNA, 17 bp, synthetic construct
  source 1..17: mol_type = other DNA
gccgcctagcaccattt

SEQ ID NO: 156 - DNA, 17 bp, synthetic construct
  source 1..17: mol_type = other DNA
ccgccaaaagctgggtt

SEQ ID NO: 157 - DNA, 18 bp, synthetic construct
  source 1..18: mol_type = other DNA
gcgcgtgaggtagtagtt

SEQ ID NO: 158 - DNA, 20 bp, synthetic construct
  source 1..20: mol_type = other DNA
gcgcgcgttcaagtaattca

SEQ ID NO: 159 - DNA, 16 bp, synthetic construct
  source 1..16: mol_type = other DNA
tcgcgaggggtgctat

SEQ ID NO: 160 - DNA, 15 bp, synthetic construct
  source 1..15: mol_type = other DNA
cgcgtggcagtgtct

SEQ ID NO: 161 - DNA, 18 bp, synthetic construct
  source 1..18: mol_type = other DNA
ttccggaggcagtgtagt

SEQ ID NO: 162 - DNA, 16 bp, synthetic construct
  source 1..16: mol_type = other DNA
gcggcggagtggggaa

SEQ ID NO: 163 - DNA, 18 bp, synthetic construct
  source 1..18: mol_type = other DNA
cggcggtattgcacttgt

SEQ ID NO: 164 - DNA, 16 bp, synthetic construct
  source 1..16: mol_type = other DNA
gcgcgctatacgacct

SEQ ID NO: 165 - DNA, 21 bp, synthetic construct
  source 1..21: mol_type = other DNA
cgcgcgcgttcaagtaatcca

SEQ ID NO: 166 - DNA, 16 bp, synthetic construct
  source 1..16: mol_type = other DNA
cgcgcatgccttgagt

SEQ ID NO: 167 - DNA, 16 bp, synthetic construct
  source 1..16: mol_type = other DNA
cgcgcggatctcactt

SEQ ID NO: 168 - DNA, 18 bp, synthetic construct
  source 1..18: mol_type = other DNA
cggcggtgaggtagtaga

SEQ ID NO: 169 - DNA, 18 bp, synthetic construct
  source 1..18: mol_type = other DNA
ttcggcggttttgtgtct

SEQ ID NO: 170 - DNA, 15 bp, synthetic construct
  source 1..15: mol_type = other DNA
gcggcggtcctgaca

SEQ ID NO: 171 - DNA, 17 bp, synthetic construct
  source 1..17: mol_type = other DNA
ggcggaaaagctgggtt

SEQ ID NO: 172 - DNA, 20 bp, synthetic construct
  source 1..20: mol_type = other DNA
ctttatggagtcgtatccag

SEQ ID NO: 173 - DNA, 15 bp, synthetic construct
  source 1..15: mol_type = other DNA
gccatggtcggtcgt

SEQ ID NO: 174 - DNA, 17 bp, synthetic construct
  source 1..17: mol_type = other DNA
tccatgggttgtcgtat

SEQ ID NO: 175 - DNA, 16 bp, synthetic construct
  source 1..16: mol_type = other DNA
tgagagggcgagtcgt

SEQ ID NO: 176 - DNA, 15 bp, synthetic construct
  source 1..15: mol_type = other DNA
ggaacctggtgtcgt

SEQ ID NO: 177 - DNA, 16 bp, synthetic construct
  source 1..16: mol_type = other DNA
gggcatatacacgtcg

SEQ ID NO: 178 - DNA, 17 bp, synthetic construct
  source 1..17: mol_type = other DNA
tgccccgaggtcgtatc

SEQ ID NO: 179 - DNA, 17 bp, synthetic construct
  source 1..17: mol_type = other DNA
gtgcaggtaggtcgtat

SEQ ID NO: 180 - DNA, 17 bp, synthetic construct
  source 1..17: mol_type = other DNA
acagggctatgagtcgt

SEQ ID NO: 181 - DNA, 21 bp, synthetic construct
  source 1..21: mol_type = other DNA
agtaataatgcggtcgtatcc

SEQ ID NO: 182 - DNA, 21 bp, synthetic construct
  source 1..21: mol_type = other DNA
tggtttacagtcgtatccagt

SEQ ID NO: 183 - DNA, 17 bp, synthetic construct
  source 1..17: mol_type = other DNA
tcgcacccgtgtcgtat

SEQ ID NO: 184 - DNA, 18 bp, synthetic construct
  source 1..18: mol_type = other DNA
cagggctatcagtcgtat

SEQ ID NO: 185 - DNA, 17 bp, synthetic construct
  source 1..17: mol_type = other DNA
gtcggtgagtgtcgtat

SEQ ID NO: 186 - DNA, 21 bp, synthetic construct
  source 1..21: mol_type = other DNA
ctgatgttgagtcgtatccag

SEQ ID NO: 187 - DNA, 17 bp, synthetic construct
  source 1..17: mol_type = other DNA
gggcattggtgtgtcgt

SEQ ID NO: 188 - DNA, 16 bp, synthetic construct
  source 1..16: mol_type = other DNA
gctccttgagggtcgt

SEQ ID NO: 189 - DNA, 21 bp, synthetic construct
  source 1..21: mol_type = other DNA
gaaatcggttagtcgtatcca

SEQ ID NO: 190 - DNA, 15 bp, synthetic construct
  source 1..15: mol_type = other DNA
gagagggcaagtcgt

SEQ ID NO: 191 - DNA, 19 bp, synthetic construct
  source 1..19: mol_type = other DNA
tgtgctgttgtcgtatcca

SEQ ID NO: 192 - DNA, 20 bp, synthetic construct
  source 1..20: mol_type = other DNA
ggataggtgtcgtatccagt

SEQ ID NO: 193 - DNA, 20 bp, synthetic construct
  source 1..20: mol_type = other DNA
ctgtgattgagtcgtatcca

SEQ ID NO: 194 - DNA, 18 bp, synthetic construct
  source 1..18: mol_type = other DNA
tagctggttgtgtcgtat

SEQ ID NO: 195 - DNA, 18 bp, synthetic construct
  source 1..18: mol_type = other DNA
tagctgattgcgtcgtat

SEQ ID NO: 196 - DNA, 18 bp, synthetic construct
  source 1..18: mol_type = other DNA
cttccatgagggtcgtat

SEQ ID NO: 197 - DNA, 18 bp, synthetic construct
  source 1..18: mol_type = other DNA
cggcctgtgtcgtatcca

SEQ ID NO: 198 - DNA, 20 bp, synthetic construct
  source 1..20: mol_type = other DNA
ctgcctttctgtcgtatcca

SEQ ID NO: 199 - DNA, 17 bp, synthetic construct
  source 1..17: mol_type = other DNA
ccaggataggctgtcgt

SEQ ID NO: 200 - DNA, 19 bp, synthetic construct
  source 1..19: mol_type = other DNA
taggaccgtgtcgtatcca

SEQ ID NO: 201 - DNA, 16 bp, synthetic construct
  source 1..16: mol_type = other DNA
tgttgcccagggtcgt

SEQ ID NO: 202 - DNA, 21 bp, synthetic construct
  source 1..21: mol_type = other DNA
tgtatagttgtcgtatccagt

SEQ ID NO: 203 - DNA, 18 bp, synthetic construct
  source 1..18: mol_type = other DNA
ccattccccaggtcgtat

SEQ ID NO: 204 - DNA, 21 bp, synthetic construct
  source 1..21: mol_type = other DNA
tccacggaagtcgtatccagt

SEQ ID NO: 205 - DNA, 18 bp, synthetic construct
  source 1..18: mol_type = other DNA
gagagggcgaagtcgtat

SEQ ID NO: 206 - RNA, 22 nt, Homo sapiens
  source 1..22: mol_type = other RNA
tagcttatcagactgatgttga

SEQ ID NO: 207 - RNA, 22 nt, Homo sapiens
  source 1..22: mol_type = other RNA
taatactgcctggtaatgatga

SEQ ID NO: 208 - RNA, 22 nt, Homo sapiens
  source 1..22: mol_type = other RNA
tccttcattccaccggagtctg

SEQ ID NO: 209 - RNA, 22 nt, Homo sapiens
  source 1..22: mol_type = other RNA
tcctgtactgagctgccccgag

SEQ ID NO: 210 - RNA, 22 nt, Homo sapiens
  source 1..22: mol_type = other RNA
tcgtaccgtgagtaataatgcg

SEQ ID NO: 211 - RNA, 22 nt, Homo sapiens
  source 1..22: mol_type = other RNA
tgtcagtttgtcaaatacccca

SEQ ID NO: 212 - RNA, 23 nt, Homo sapiens
  source 1..23: mol_type = other RNA
tgagtgtgtgtgtgtgagtgtgt

SEQ ID NO: 213 - RNA, 22 nt, Homo sapiens
  source 1..22: mol_type = other RNA
caatcactaactccactgccat

SEQ ID NO: 214 - RNA, 24 nt, Homo sapiens
  source 1..24: mol_type = other RNA
agcctggaagctggagcctgcagt

SEQ ID NO: 215 - RNA, 22 nt, Homo sapiens
  source 1..22: mol_type = other RNA
tccctgagaccctaacttgtga

SEQ ID NO: 216 - RNA, 22 nt, Homo sapiens
  source 1..22: mol_type = other RNA
taacactgtctggtaaagatgg

SEQ ID NO: 217 - RNA, 24 nt, Homo sapiens
  source 1..24: mol_type = other RNA
ttaatgctaatcgtgataggggtt

SEQ ID NO: 218 - RNA, 22 nt, Homo sapiens
  source 1..22: mol_type = other RNA
aactggccctcaaagtcccgct

SEQ ID NO: 219 - RNA, 25 nt, Homo sapiens
  source 1..25: mol_type = other RNA
agtggttcttaacagttcaacagtt

SEQ ID NO: 220 - RNA, 22 nt, Homo sapiens
  source 1..22: mol_type = other RNA
ctgtgcgtgtgacagcggctga

SEQ ID NO: 221 - RNA, 21 nt, Homo sapiens
  source 1..21: mol_type = other RNA
agctacatctggctactgggt

SEQ ID NO: 222 - RNA, 22 nt, Homo sapiens
  source 1..22: mol_type = other RNA
tagcaccatttgaaatcggtta

SEQ ID NO: 223 - RNA, 23 nt, Homo sapiens
  source 1..23: mol_type = other RNA
tccctgtcctccaggagctcacg

SEQ ID NO: 224 - RNA, 22 nt, Homo sapiens
  source 1..22: mol_type = other RNA
taatactgtctggtaaaaccgt

SEQ ID NO: 225 - RNA, 23 nt, Homo sapiens
  source 1..23: mol_type = other RNA
tgagaactgaattccataggctg

SEQ ID NO: 226 - RNA, 22 nt, Homo sapiens
  source 1..22: mol_type = other RNA
tctcccaacccttgtaccagtg

SEQ ID NO: 227 - RNA, 22 nt, Homo sapiens
  source 1..22: mol_type = other RNA
cgaatcattatttgctgctcta

SEQ ID NO: 228 - RNA, 22 nt, Homo sapiens
  source 1..22: mol_type = other RNA
tagcagcacgtaaatattggcg

SEQ ID NO: 229 - RNA, 23 nt, Homo sapiens
  source 1..23: mol_type = other RNA
taaagtgcttatagtgcaggtag

SEQ ID NO: 230 - RNA, 23 nt, Homo sapiens
  source 1..23: mol_type = other RNA
tagcaccatttgaaatcagtgtt

SEQ ID NO: 231 - RNA, 23 nt, Homo sapiens
  source 1..23: mol_type = other RNA
cagtgcaatagtattgtcaaagc

SEQ ID NO: 232 - RNA, 22 nt, Homo sapiens
  source 1..22: mol_type = other RNA
tgtaaacatcctacactcagct

SEQ ID NO: 233 - RNA, 22 nt, Homo sapiens
  source 1..22: mol_type = other RNA
tggcagtgtcttagctggttgt

SEQ ID NO: 234 - RNA, 24 nt, Homo sapiens
  source 1..24: mol_type = other RNA
gttccacactgacactgcagaagt

SEQ ID NO: 235 - RNA, 17 nt, Homo sapiens
  source 1..17: mol_type = other RNA
accccactcctggtacc

SEQ ID NO: 236 - DNA, 52 bp, synthetic construct; identical to SEQ ID NO: 118
  source 1..52: mol_type = other DNA

SEQ ID NO: 237 - DNA, 50 bp, synthetic construct; identical to SEQ ID NO: 35
  source 1..50: mol_type = other DNA

SEQ ID NO: 238 - DNA, 52 bp, synthetic construct; identical to SEQ ID NO: 36
  source 1..52: mol_type = other DNA

SEQ ID NO: 239 - DNA, 50 bp, synthetic construct; identical to SEQ ID NO: 34
  source 1..50: mol_type = other DNA

SEQ ID NO: 240 - DNA, 52 bp, synthetic construct; identical to SEQ ID NO: 33
  source 1..52: mol_type = other DNA

SEQ ID NO: 241 - DNA, 52 bp, synthetic construct; identical to SEQ ID NO: 121
  source 1..52: mol_type = other DNA

SEQ ID NO: 242 - DNA, 52 bp, synthetic construct; identical to SEQ ID NO: 5
  source 1..52: mol_type = other DNA

SEQ ID NO: 243 - DNA, 52 bp, synthetic construct; identical to SEQ ID NO: 126
  source 1..52: mol_type = other DNA

SEQ ID NO: 244 - DNA, 19 bp, synthetic construct
  source 1..19: mol_type = other DNA
gcgcgcgtagcttatcaga

SEQ ID NO: 245 - DNA, 18 bp, synthetic construct
  source 1..18: mol_type = other DNA
ttgccgcctccttcattc

SEQ ID NO: 246 - DNA, 17 bp, synthetic construct
  source 1..17: mol_type = other DNA
gccgcctcctgtactga

SEQ ID NO: 247 - DNA, 16 bp, synthetic construct
  source 1..16: mol_type = other DNA
ttcgcgctcgtaccgt

SEQ ID NO: 248 - DNA, 17 bp, synthetic construct
  source 1..17: mol_type = other DNA
atgcctgtgcgtgtgac

SEQ ID NO: 249 - DNA, 17 bp, synthetic construct
  source 1..17: mol_type = other DNA
gccgcctagcaccattt

SEQ ID NO: 250 - DNA, 19 bp, synthetic construct
  source 1..19: mol_type = other DNA
cgcgcgtctttggttatct

SEQ ID NO: 251 - DNA, 15 bp, synthetic construct
  source 1..15: mol_type = other DNA
cgcgtggcagtgtct

SEQ ID NO: 252 - DNA, 21 bp, synthetic construct
  source 1..21: mol_type = other DNA
ctgatgttgagtcgtatccag

SEQ ID NO: 253 - DNA, 18 bp, synthetic construct
  source 1..18: mol_type = other DNA
ccggagtctggtcgtatc

SEQ ID NO: 254 - DNA, 17 bp, synthetic construct
  source 1..17: mol_type = other DNA
tgccccgaggtcgtatc

SEQ ID NO: 255 - DNA, 21 bp, synthetic construct
  source 1..21: mol_type = other DNA
agtaataatgcggtcgtatcc

SEQ ID NO: 256 - DNA, 16 bp, synthetic construct
  source 1..16: mol_type = other DNA
agcggctgagtcgtat

SEQ ID NO: 257 - DNA, 21 bp, synthetic construct
  source 1..21: mol_type = other DNA
gaaatcggttagtcgtatcca

SEQ ID NO: 258 - DNA, 22 bp, synthetic construct
  source 1..22: mol_type = other DNA
tggcggtcgtatccagtgcgaa

SEQ ID NO: 259 - DNA, 18 bp, synthetic construct
  source 1..18: mol_type = other DNA
tagctggttgtgtcgtat

SEQ ID NO: 260 - DNA, 18 bp, synthetic construct
  source 1..18: mol_type = other DNA
tcgtgtcgtcgtcgtacc

SEQ ID NO: 261 - DNA, 22 bp, synthetic construct
  source 1..22: mol_type = other DNA
ctcgtcgtgtcgctccttcatt

SEQ ID NO: 262 - DNA, 18 bp, synthetic construct
  source 1..18: mol_type = other DNA
caccggagtctggtcgta

SEQ ID NO: 263 - DNA, 19 bp, synthetic construct
  source 1..19: mol_type = other DNA
tgagtaataatgcggtcgt

SEQ ID NO: 264 - DNA, 15 bp, synthetic construct
  source 1..15: mol_type = other DNA
ctgccccgaggtcgt